SEQUENCE LISTING (108 sequences)

SEQ ID NO: 1 - DNA, 3339 bp, Homo sapiens
agtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 2 - DNA, 39928 bp, Homo sapiens
agtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatggtgagttctcatggctctattgggctatttgttgcctcttaaagattagattactgcccgtgaaactgaaaagggaaatcaaaaaaaatgctctgagttgtgagattggatgtttgcctccatatccctgattttggagtcccagataactaaacttaattgaccctggggttcattcagaaaattgttacaaaataattcactggtctcaatccagatgcttggaaacaagagaatattctttataaagttaccaccataattctcatcacagtcaggatttcaaagcatttcatggaaatgccataagttattgagttaataatttcctttagcctacaatatcaataggaatatctaaagattctctacaaatgatatattaactgaaaaatgcaactggaggcttagtgaaagaactagtaattaagctaatcagggcttgggtgaggctggacttgggaattcctttcctctttgtcacagacctccaagggactccaaaaggtcacagaatccaggcagagccctccaaaacaaaaacaaaaacaaacaacaaaaaaactatgattaaaccagttctgacaaatatcagaatgctctgggaaagccagatgctttaacaagtgcaaggatttaggaatttcttctctcacagatcccaaaacagtatccttaagtggtgatccttatgtcaagctgtgagaatatacctcaaaataatagaaggcatccaaacttgatcagtatagtgtcataaagctgctgatgtagaagtgtggagaagtccatcagatagagatatggaaaaaaaagcttggttaaaacaggtttccaaagttctctccctttctctgccattgtggtatgtgtggttgaccatttctcatcatgttttcttacaagactttcaggatcaagcaattcctcgccaaaaaacaaaatcaaggacacgttgtagctatttcttggtgagttaccagaagctgtgaagagagagcatgaggcccatacttttttttttttttgaagggtctcgctctgttgcccaggctggaatggagtggcatgatcatgattcattgcagcttgacctcctgggctcaaatgatcctctcgcatcatttaacatttttttttgtagagatgagatctcactttgtctcccaggctggtcttgaactcctgggctcaagtgatcctcttgcctcaggctcccaaagtgctgggatgacaggcatgagccactgcacctagcctaccacacttactctttgcgtggtctctagagcttcttcagcccccctgctgccagggtccctagggtcttgggcatgccccatttctgaacctattcctgtctccagggatagagtcacagtgaactcagaccagggtaattctaaactgctattctgtatatacccaaggaaatttcatccctgtctctaatgagtctgtcccatgccccttaatacacatgaacacctacacacaggtacaagtcggggaagatcactgtgcagcacataaaataaaattcaggggcgagcactttttttcccatgagataaagttaggaaaaaataaaaatgttcatacagattaaaaattttaaatgttaaaatattaaacccccaatctaaaataaagaaaaaataatttcttaaatcctaagcttgagttatggaattcacattcttataatttacattacctattttgttaaatggaagacttttttttttaatatgcaagtacctgggcatatttagaaagtaaccaaggcagcttttgtctttgaacctgatgcggttttgacagctctatctggaaattgagccagcatatgggagccaagggtgaaagttttcctgggagatttgggctccaaggtactggatagatgctaaagattccattcagtttttcattttctgtcttcatggaactctcacacctacaggggaagaaatggtttagaggttactaaaagtatatagcctggtttaggtgatagtgagctatagaaaaagagaaaataagttgtcaatactttaaatctcaaacaacttcttacaaaaggattttaatagtttaaatacaattatcacaagttgggtaaagacaaccagacctatggcctgataaatcatgtatttaatttatgcatatagttagggccaaaaagagcagagtatctttttgaatgttttaaaatataatctaggattttatgcctacagtccttgaaattacaaaggatctctctcaagttgggaatatgctatccattgcagttacggatgggatttagagactgttttaatttcttgtgattatctcaaacaacgtgttccttactcccaaaactcccttgtatggctacagaaactgttgcagttttagttgcttctcccctgcctttgcccagaggtagtgtggaatgccaccaattttatctgtaatggttcctttcaagcctggggctatgtaagctgctatatatgcttcattccattcatcattcagtctaaatatagctatcaaccactaaattatctgataggtatgcatgaactgactgattctttgcataactactaagctatctgttttatttaggcttgggagccctagggtaaggatacggaataaattgtaaatgatattaaaagaatagcttggcaaggaataatacctcctcaggggccaaaatgcaaggcaacacaaagtgatttttttggtgatataattataacacatgtagtatggtagtgataactaaaataaaattggatagcctcaaaattcctacatgtagtatgataaacaatgttctaagactgaggcctttgggaggtagtgagttatttcctatagatagagagtaaaattattcctataatttggtatgaaagttttaccagaaattttgcttcacagtaaatcatctggattgggtgaagccaggtagggattgagaagtgagtcaggtgggttgcatagatgagtttgtgctgcagaccaaactcctcttcccatagctcacttcttcccactcctgcatgttagagagaagactagaatgtgtcaaatacaacctataggataatagttcagagcattagcttcggaagtaaacactaggttgatgccctggttcttctaatttctagctgtgtgacctagggaaagttacctgctgtgtgcctcatttcccttatttgtaaagttgagacaataacaactccctcacaaggttgttgtgaagattaatgaaaaagcagtttctgagaggttgtgagtgttcagtaaagaaagaaattgctttcctttttttttttttttgagacagattctcattctgtcattcaggctggagtgcagtggcgtgatcatggctcactgcgcctcgacttccccaagctcaggcaatcttcccacctcagcctcctgagtagctgggactacaggtgcgagccaccacacctggctagtttttgtattatttgcagagatggggtctcgccttgttgcccaggcttgtctcaaacttctgggctcaagcgatcttcccacctcaacctcccaaagtgctgggattacaggcatgaaccactacacacagcctattttccattttcactgccattattttctctgtctcttgtgtggctcattactgccacttctgtgcaccatttccatcatgtcacatgcgtagtcaaaaccctcagtgactgcctaattctcagagggtaaaagtgaaattccttggcctggaaagtgcaatcatgcctgaagttctacctgtgggagctgtactagttatctattgtgtaaaaagttaccacaagcttagcagattaaaacaacacatatttaatatcccacagtatctgtgggctaggagtccaggcacagttcagctggatcttctgcttctaagtctcatatggttgcattcaaggtgctggctagagctatggtctcttctgaggcttgactggggatggatatacttccaagccaatgcaattactcagaggccgtctttaattatttgccatgtggccttttccatagggtagctcacaacgtggcatcttgctgcaacaagtcagcaaaggagagagtcttctatcaagattaaagttaaaatcttatgtaacgtaaacatgacatcctgtcacctgtgttgtatgctattggttgaaagtaaatcacgggtctgctcatacttatggggaggggatcacacaagggtgtgaccaccaaaggcagagatcatgggagcattcttagagtctgtctgccacagggccaggatcaaatgctggtggctgattgccagctccactgtttatttctctcagccgctttctgtatgtacaaaaaaggagagagagtgattttctacctcatagtgttgttaagaaaatcaaatgaggtaaattattttgcaaagtattctctactcatctgactcctacctactttcaggtctgtcttaagttgcaacttctgcatgagggcttggccaagtatggtcacctgcatgaatttcctctttcttgtaacagaaactgcttggcccactcataatttatacattattatatactcttatgtttcattaatgaaacatttataatgcatatcttatctgtctgataaaatggaaagcaatctaaggacaacgacttttcccctatacataacaaaatataaaataaatcataacataataaataataaataaataataaaataactaacatttgttgaatgattaggatgtgttaggcactcttgtaagcatttcatttttttgttacttcttcaaatccttacaacagtccttggaggtaagtcctattattactttcattttgcagataagacaactgtggctcagagaggctaaggtacttgagtccaggagcagaattcttactttcctcaaacgttatctcttttgtgtggtctagggcagtacaatataccaagtagttgctcattttggtctttggtaaatggaaatgaatttgtaagttgagaaaagactacactacagtagtttggagaagtccactttccaactgatgttcaagattcaggagggggggaaatgtgtcctggcatcatcacttgcttgatgatttcttttgctggtagaaaatcaccatggctaccaatggccattttctgggaagccttttttgggaatcctaacttaagatcaaaatccctgggctaagtcacggcagctaggaggttactgtgaaccggttctcatttcttatggtttccattgttattagggggctgtattctcattaagtccactctgagctctactggattctctttttagagcactcgtctttgaattcttggccaactcatctatgtcacagcactaaggctataaattccagggccccacaatgtggattatctgtgagaattcatttccagttctggggacaagacccttgggaaaaccaggcaataggccagagagagacagagagagagagagtggtcaaaagtggcctggtcactcttaacctaaaccttgaccttcagcggagtagaggactaaatcactcaggaaatattaaatgtttcaccagatattatttggactatatcttctgttctatctcttcaagcaatgccattccaacttcaattttcttttctgtcatttcagaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacgggtacgtttgtgaacattttagcattgatcccaagagttgaaatatttgggaaatattaaacattaatatcacccacagcagctgcctttatttgaaaaaatagtaatgctgatgttagaaaaatctctctctatgcctaggtaaggtgtccatataatttattattcaaactggaatctttgagagtgaaagacactggtaattattatattcaaacacagagttaaactgagattgtccctggcactgtggtgtgtatggacaccccctagtgttaggtcaccttcctgaaggatcaggcagcctgaagaatacagttagtttatgtcagtggttctcaactgggggtgatttagccctccggggatatttgacaatgtctggagacgtttttggttgccacaaatggggatactgttgacatctagtgggtagggatgctgcagaacatttcacaatgcatgggatggcccccaacaacaaagaatgatcttgtccacaatgtcaacagcaaacttttttttctgtaaaggaacagatgacagtaaatattttaggttttgtgtatttaggtattgtctgtgttgcaactagtcaactgtgctgttgtatcaggaaatcagctgtagctaatatataaatgaacaagtatggctgtgttcaaataaaactttacaaaaacaggcagcagacaggatttggtctgcagaacacagtttgcagacctctgatatagcctattgaaatatttttaacctaaaatatttagctatttaaaaagaaagtcttctttctcagttgcttgtcctctggaactttgtatgttctaatctgtgaatataaaaataaaaatgcatcaaaaaggcaaagataaaaccatgtgaaatgtatctttctacaagttaacatttatttttctgcattctttgggtccaattatccaactaattatcaatgcactttactttatccattctatgtttctccattttttcccccaacaaggacactataagaaaccttgcccattcgcatatagacaatctggtctagcctaggtggaacctagaaattctattttttaaaaaatccccagataattctaatgtgtagccaggactgaaaaccactgaaatgcatgtaagagcttaaagtgtttgggaagaggagggactcaaggttgcttagatttgatggagaggaagccgtggagattgaagaagagctggagagagaccatgaggggttttgtgccatggtaaggaatttaggtttgttcctttggactatgggatcgtgtggaagttttaaacataggatctatttcatgttttaggatgactctcaaggcagaaccaaagggaaataggagaaggagaaacagatcctttaatccagtgcttcttaaatcaatctgagggagagaaccatttccttccttccttccttcctttcttctcttcttccttccctccttctctcctccctcccgtctttcctttctccctcctttattcctttatttccaatacattgcagatcaatacttgcttaagttcattgattacatgcttagatgtcatggaaatatcaaattgttctccaaagtttataaacttttattctcttaattcctgtacttatcacagaatggaaacaaactgtcacttgcactggtccacggattacacctggagtagcactgcttcactatatcaccttcactttatctgcagggtaaagtttaaaaactttggtatggcatagacggtccctcgtgatctggcccctgcttgtctctccattctcatgtctttcatttcccctaatgaacaccattgctccagtttcattcaacatctcactgtcccctgagaatgccttgtcttttccaacctccttccatcttttagactcttccctctacctcccttctctacatgaccagtgcctacttcttctccaagacacagctcttatcttcccttctctttggacccattcttgaatctcctggttattagttattaatttctccctgctcctatactaccgcatcactttttggtgacagttatagcacttgtcattctgttatgtattaatagttattgatttcatggctgcctcactgtcctatgactttatggtctttgacttgttcacttcagaattgtcagagtgtaggacatattccaggttcaataaatgctgaatgaaacaaaaaatgcacctctctgtgtatgtgtggataagaaaacttctttgggttttggtagatctggttatttcaattactccatatatatttattatgtgcttatagtactttgtgctagtcgacagtggggaaacttaactgggctattccttctccacatacagaaagatacatggcaaagaagtaaattgctgtattttgagtaatcaaaaaatcatgtggtcaaaaggatatctttatattagcattctcttcagcaaaatttccattgttaacattgtttattatctttaatttgcagttgaacacaattctaaatacaatgagcaccatctacagtactggaaaagtttgtaacccagataatccacaagaatgcttattacttgaaccaggtaggctactaatttttagtagtgattatgaaatttacttttctctcagattttaaaaatgattgcagatatgtgtgtttcaacacatagatgctcttttaacttttagtaaaactttcttgtaaattgtatcacatttacttaatttgatcaagtcagtaagtaagtcatttcagtggtttattttctctgcagacatattttggaactataagggcaaggacatgaatgaatcatgagattttatttaaccactttatgacctgatgtctgccaaccttacttttaattctttaaagttcatgtagttctttacaaataaaatttttacaaaggcatcttttcttgtctttgtgtgctttgggataacaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcgtaagtttgctgatctgtagaggtcctgaagctctctgtgtggccacggggctatttcttctgcattaccaagcctgccctttgatatcagggatacccctcaattaacaatcatgtcctcaacaattcagattacctgttttcaaaagaaattactggggaaaaagaaaaggtgcactggcattctacaattcaagaagctcaagaatgagactcaactacaaggcctaaagtaaattatccaggaattatgaattataagtaatctaggaagatctcaggcccctaaaagttcccgagggagtctggggattaagggggggaatcccatttctcaggatgaaagacagagtcactgccttttgtctaaagcccccacagttcactttcaaaggtcatttgtgtgtctaccttagggtgataccccagaaagaaaaaatatcagattagtttaggctggaaatgtattattttctgcagttgtaattatatctcagattatgtggtcataaaaaatcttaacataggaaggaatttccatatctcttaacttattctcttccctgaatagtgagctgaagtcatggaatcatgattcacattttgctttgatgatttactgcccactgaggttgagtaaggaagaatttttttcccgcatttgttgttttaacatttctttcactgccacttaaattcagtattcttaagccttaggcaagctggagacaatagcttatcaccatttttaaaacaatatcctgcatgtcctttcatccaaatatttatttctctcctcatcttcctgatcattaattgccaatgtgaatgtaaaaataaaaattattaaattatgtttacaaactttattagctaatatcctgacacaagcttgtgttttctgatggtgagatactaaaggaagcacatagaaacatgctattgatacatataatttgcaaacaaacacaggttcactgtggcaaagtctaagatgagttgggtctgatctaaaaataaaaggccatgaacaaaagaagaatttaaaaacttagagatccagtcacatattttaaagctaaagatacaaactgatgtatcaagagagtacacacccagggttaagagactaggaatgtgcttctttcgcttaccagctgtgtgatcttggacaagtgatttaaccactctaagcacaggttgttggtgggaatataaactggtgaagctgttatggaaaaatagtatgaggattcctaaataaattaaaaatagaattaccgtatgatccagcaatccctcttctgggtatatacctaaattagatgaaattaccacttcataaagatatctacactgccatgttcactgcagcattattcgcaatagccaagatagggaaacaacctaagtgtttgtcaagggatgaatgaataaagaaaccgtggtgggtatatggaatgggatattattcagccttaaaaaagaggagatcctgtcatttgcctcaacatggatagacctggaggacattatgctaagtgaaataatccagacataaaaagaaaaatatggcatcatctcactcaaaagtggattctacaaaaaaatcaaatatacagagatagagtaatagtgattaccaggggtgaggtggagcggggtgggatatggggagaagtgggttaaaggatacaaagtagcagatatgcaggatgaacaagtctaaagatctaatgtacaacataaggactgtagttagtaataatgttttgtattcaggatttttgcaaaatgagtagattttagctgctcttccatgggggggataatgcttaactatgtgagatgatggatatgttaatttgttttactatagtaaccattttattatataagtatctcataacatcatgttgtacaccttaaatatacacaataaaatttatttaaaaataaacataggctgttactctgcacagtgggagttataataatgacaatcttgtatggttcttgtgcttattaaataaaataatacatttaaactcctggcacagagcaagtgcttaaacagagattacttaatgttattattcatttgcatccctttgagttgatgtttatcacaataaaaatgtgataagcctttaaatttcttttaacctcccagcaaggctaatctatgtattttgtgctttctctctttctttcccttatgttcttccctttctctttaaaaaaaaaaacaaacagattatgaggactatggggattattggagaggagactatgaagtaaatggggtagatggctatgactacagccgcggccagttgattgaagatgtggaacatacctttgaagaggtaagcaaggaactgtacacacaaacagttgtggggcagtgtatgggaaagagggacagtgactaaaggcattctggctcattcttaacacattggtcagccattcccaggcctcataaagttatggaatattagagtttgaaagattttagagaataataggacagcccctacagatgatcagaattgcgctgctttcctccttctttcagtcactgaccattaattaccaatagaaagactataagaatgaaatattataagaacaaatggtcctttagattataagtaggaatgatcttcttgagctacctggggctggcactgtcttcctgtgttagcagagctatggactttgctttcttcctcaggacaattgatactattactttgagtaagctagtcttcatagggaagagagagttttgtgaacagaaatgtcccatggatgtttttccttcattttggccccatgcctcccctttacgtgaaccttcaaacttctgatgtgagttaccttgtcacagccatagtttggcagagctatcaaaaatggaagtaagacaaacaactccccaaaaaggaaatgggaagaagacttgaataagtacatcatcaaagaaattacacaaatggcaaataggcaaataaaaagttattagggaaatgcaaattaaagctatgatgaaaatccactccacacccaccaggatagctatgattaaaaagatggacaataccaagtattggggagaatgtggagacactggagccctcttacagtgcaggaagaaatgtaaaatggggcagtcactttggaaaacagtttggcgatttcttaaaaagttaaacacacacttaccatgactcttaggtttctatatgaaataaatgaaaacatgtgtccttatccatagaaagacatacacaaatgttcatagcagcaatattcataatagccccaaactggaaaacccagctgtccttcaactagtgaatgaataaacagactctgatatactcaaagtagccggagataggagtggaactgactacaagaaaccttttgtggttatagaaatattaaaaaactagatagtggtgatggtttcccggtagttttaatttattgaaactcatggaactacacacttgaaaatgggtggcttttatagtatgtaaattatacctcaatgtaaaaacaaaggaagtaggtaacaatcttaaaaatgaatatatattttttgcatctgcttttattttgggacctgtgttctcccaagtatgtttattggtttatgcagtggagttatcatcaagttctaggaagaaggaatacttttaaattttccagaaatttgagagaagaagaattataatttggggaaattctacttgtttaaagaatctcctatttctagtgttgtggaatggaaattagaattggttactctttgtcttaaactttcttcctgggcttttcagattaaaccattatatgaacatcttcatgcctatgtgagggcaaagttgatgaatgcctatccttcctatatcagtccaattggatgcctccctgctcatttgcttggtaagaagccccatgaattcttcgtgtacttggctctttgttatttctaacaataaaattgcctttttgagtgagccttaacataaaacttttgaaaaattctgtaagtggttagtaacattgtagaaatcgcattttgaaagcattttcttcaaccaaaagtctattttaaacaattctctattgtattactttttttgtacataaagagttattattttggctgcaattcaaatgataaaggggttatttaagataactagagaaagaattacaggctcaaatgagagtggaaatggggaaaataatttttagtattcaagttgattggctaagctcttaaggtggaatttgagattttagtatcatattgtcagagcttggttggctgggggtggtcaggggtaacagaaggttatagcttgagcgaaagcttcctaagttcattctagacttcaccaattactcttgctgaaaagtgcttggcttggaatcgtataagtgaagaatgaatgaaagtgaccatcttcccctctcccaccgcagcagctgaggccaaataaaatataaatatttgaaaatttcatgccatgcatatatagcatttctactgtagtcaaactaagttgctggaagctaatctcctccacagccatacctcgtgtctgtcagcaggagagaatgcggagaaaggacctgtggaggcttttaactctaaagtaatatggaaatccttcctacaatgtcaatggcaagcattatttagtaattcagtttctgcttaaacctgtctggtgtgatgggataatttcttcattccaatatagactaatttactgctacatggaaccaatgtaatacaattctaccttatgtggaacttaagcaacacccctctgatgccttccattcattagccctatttccatgctatgaggttacccagaatgtcaattcctattctacaagatgaccttccagatattcataaagaaccatcatataagatacagcatagtacctggtgcaatacattttagtgactattattgttattgtcatgtttaccctattttcttttgtcaagttaaatatcttatacattatcaaccattccttaaaacctcacttttctataattgttctcctttcaatgagctgtactttgtcaatgtccctcctcaattatcatgcttagagatgaacagttactccagatgaggtttgaccaacataaagaaagacatggtaccatgactacctttgttctagacactatacttctattgatttagctcaagttcacatttatgctttagaagccaggtcatactgtttgttggctcatttttggtcttttacatgtggactgtgctttagcctgatgtcacatatatgtttttatgatggataggtttttctttaaaatttaagcataggacttcacatgtatcactactaaattttatgttttaaaatataagtttagggagtacttttatttttttcagagtctcgctctgtcgcctaggctggagtgcagtggctgatctcagctcaccgcaacctctgcctcctgggttcaagcgattctcctgcttcagcctcctgagtagctgggattacaggctcaagccgccatgcccagctaatttttgtatttttagtagagacagggcttcaccatgttggccaggctggtctcgaactcctgatctcaagtgatctgcctgcttcagctctgcaaagtgctgggattacaggagtactcttcatgaagtttgttgccccatttttttggcaccaggattctggtcctgctcttggattgactttttctagatacatcttcttttttatggtccctggccttggaatacaacttaacatttgtttgagatgtttacaaagtcaccaagttaagtacacgaatataggaataaaagtggaatttagttaccagttagtagaagttcctgaatgatgatcttatatgatctcttttgaagccaacactaggaattactaacagcttaatttgtaatattttgtaccatggatggtagattatagaaatctctttctaatttacagtgtcaatgatggtccatatatttaataaattatgtctacatttctgctgtgttgtcatatactaacagattcttttttaaatataggtgatatgtggggtagattttggacaaatctgtactctttgacagttccctttggacagaaaccaaacatagatgttactgatgcaatggtggaccaggtaggaaaaagagcccttaaaaactaaatctaaattctcaacttcatttatttttatgtcatccttctatttttatttttatgtgaaaccatgtaaataaaaaatttaagctaattgaaatttatttcttatgtagctatagtttcatggaaaagcagtttacaatggcctcagataacttgaattgcaggtgtagtattgatctcattgaaaggtatttgaaatggagattacctgagttttccattaagcggtaagatgtagaattgtcaactggttggtatcactcattccaaagtttgtcattgctaagtaaccagccagtaatctctatggatagtaaaaacaagaatattatgaggggatttttgcatatttaaaaaattattagtgttaaaaatattacaagaatctttaaaacaattttaagtaacatctttattactactttggtttaattatttaaatgtgctaggttcctcaaaacacaagtaagtacatagcccatggacccaataaagcaactacacaatcgagactacaaagcaaccagctaacaacagcatgacaggaacgcttcacctaacagacagagagtggcaaattggagagaaaaacaaaacctaaccttctactgtcttcgagattcatctcacgtgtaatgacacccatgggctcaaagtaaagtgatgcagaaagatctatcatgcaaatagaaaacaaaaaagagcagggggctgctattcttgtatcagataaaacagactttaaaccaacaacagtaaaaaaggacaaagagggtcattacataatgataaagggttcaatttaacaagaagacttaattatcctaaatatatatgcacccaacattggagtatccagattcataaacatagcacttttagacctaggaaaagacttagacatccagaaaataatagtgggggacttcatcaccccactgacagtgttatacagatcattgaggcagaaaactaacaaagaaattctggaaattctggacttaaacttgacacttgaccaattgaacctaagagacatctatagaatattccacccaacagctatggaatatacattcttctcatctgtatacgaaacatactctaagactggcaacatgctcagtcataaagcaagtcttaataaattcaaaaaattaaaattatgccaaacatattctcagaccacagtagaataaaaatagaaatcaataccaggaggaactctcaaaaccatgaaataacctggaaactgaacaacttattgctgaatgacttttgagtaaacaatgaaattaaggcagatatcaaaaaattctttgaaacaaatgaaaacagagacacagcataccaacaacatatctgggatgtggcaaaagcaatgttaagaggaatgtttatagtgctaaatgcctacatcaagaacctagaaagatatcaaattaataatctaacaccacacttaaaggaactagaaaaacaagaacaaactagttccaaagctagaaaaaaagagataactaaaatcagggcagaattaatgaaattgagacctaaaaaatcatacaaaggatcaacaaaatgaaaagtcagttttttaaaaggataaacaagatggaaagacctctagctagattaacagagaaagagagaagatccaaataagcacaatcagaaatgacaaaggtgacattgcaactgatcccacagaagtacaaaccttcctattgagtactatgttcatatctggttgataggaccaatagaaacccaacgtcagtatcacacaatatacccttgtaacaaacctgcacatgtactccctgaatttaaaattaaaattaaaattaaaattaaaaaattatcaggccgggcacggtggctcacagctgtaatcccagcactttgggaggccgaggtgggtggatcacgaggtcaggagatcgagaccagcctggccaacatggtgaaaccccgtctctactaaaaatacaaaaaaaaaaaaaaaattagctgggcatagtggtgtgcgcctgtagtcccagctatttaggaggctgaggcaggagaatcacttgaactcgggaggcagaggttgcagtgagccgagatcatgccactgcactccagcctgggtgacaaagtgagactctgtctcaaaaaaaaaaattctctgtgttcccttctgttgatgaaatggtattatagttataattatgtatatttgttcattcaactaaaattctacgtttctgagtagaatatctaatcatatttctatttgccttataaaatttcttcagataattgcaccataatttactatttgtgcattgctgaacctttgcttgttaatagttttacatctgtaagaagcaatgcaatgaacatcttcacatacataacatactcctgggaaaagtccactgcagaataaaaagtgaaactgcaagggcctctaaactcttgtagctgttgtagctccactttgaagttgagcagcagtatagggccatcttgatgtagatttttagtctttctaaagggtttttacacccgtctcttctattgtcatctctgactcaggataggtattgtgacagctccatgctataggtgaggaaactgaggctcaggtaaatgatttgcccaaagtcacaaatcacacagcctcattctttctgttttttttgtaccaattaaccatcctcacctgcccctgccaccttcccactactctttccagcctctggtaaccatccttccactctgtatgtccatgagttcaactcttttgatttttagatgccataaataagtgagaacaagtaatgtttgtctttctgtgcctggcttatttcacttaatataatgaccttcggttccatctacgttgttgcaaatggcaggatttcgtttttttatggctgaatagtattccaatttgtatgtgtaccacattttctttatccattcatctactgatggacacttaggttgtttccaaatcttggttattgtgaatagtgctgcaacaaacatgggagtgcagatatctcttcaaaatactgatttcctttcttttgggtatatacccagcagtggaattgttggatcatatggtagctctatttttacttttttgaggaacctccaaactgttttccatagtggttgtactaacttacattccttccaacagtgtaggagggttcccttttctccacaccctcatcagcatttgttattgcctgtcttttggatataaaccaaaaaatttttcataaatataatttgggtggctgcacattattctatttttcaaaggtatcattatttatttaaccattctgatgttcgtttcaactttttctctattaaaaataattctcaatgtaattgttgaatctaatttctgataattttttaggttagttccttagagacagcattactgagacaaagagaagaatttcaaatgctatccgtgtgtgttatcaaattaccttttggaaaggttatgccaatgtatatatccaacagaaataatgctgctattttaaaaatgacattagcacttttttccattttaaaattttattgtaaattgacatttattattgtatatatttatggggtacaaggtgatttcatgatttatgaatacaatgaaaaataaataaagctaattaacatatcaattacttcacatactttttttgcagtgagaacatttgaaatttactctcttagcaatttcaaaatgtacaatgttctattattaactatatttactatgctgtgcagtagatctcaaaaaaataaaaccaacttattcatcctgtttgagactttgtacgctttgaccatcatctccccgttgcccccaccccatagcctctgtaaccaccattctactctctgcttctaccagttccattgttttagattccacacataagtgagaacatgtggtatttgtttttcggtgcctggcttatttaatttaagaatgctgttgttttgatgctggaattaatactgttctcttttcccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggtagtggggctgatacttacacaactgatactaaatgggagacaacagaggcaggctgaagtttaacttgaattctttctctgcttttctgagcacagaaaagataagttaattctccttgaactaaggctgggagtccaggagagcacatttgggttttcccaggaaaagaggattgccagcaggctctaacttgaacctgtggacaatctgctttcttggaaatatcatgcatccattggcgttgtatggtaatggggatggttgcacccatatcaggaaagatttggtgctaagatttttggaagatggacatatgaatcacacattgatcttcccctccatgtttcactcttgtgaatgagacagaataggtgaataaatacgcttggatcgaagggagaaagtggtacatgtcgtaagagagacacagggcatgctctatggagaactggaagaaaactgaccacatttgcaataggagataggatcagaccgtgctttacaagtgggatttgaattaggtttggaaagacaagaaggattcagatacacagagtcgggaggaggacccaagctgtgagaacagcaggatcaaatacagagaggcaggacctgacctgcatactgaagtcggcaagttaggctagaatgagaaataagtgaaggagagtttgtgtaatgtggcagaatgagcacaggcttcagaatcctaggtgtgtcacttaatgactatgcaaccttggacaaggtatttaactttctttggtttcagtttccttattttataaagtagaatagtaattcccaggttgcaggcttgtgagagccttaggttggattccctagcttgaaaaggagatcgttttacaagtgcttcattgaggagagctctgaggcagaggggaatgagggaagcaggctgggacaaaggagggaggtaagtaagaatgtgatctttactggaaacttggcctcagccttatcccatgaagctctctggagcataacttgagccacagactaggccacacctcaagacaagggggctagccttttatatcaggcagtctttggctgtggactgccccagagtatgggggagcataacccccatttagccaaaggcagtgaagggagcagctgtaggcccttatcagctgtgactcaggcagctaggggatggtggataaagtcttggcaggcccgcaccagcattgactagggtagggattggatctacagtccttttctgtaaagggccagatagtaaatagtttaagctttgcagatctctgttacaactactcagctctgcctttgtgacatgaaaacagccatagacaatacacaaaccaatggatgtggctgtgtgccaataaaaatttatttataaaaacaatggctggccagcaggccatagtttactgatgccagtactagagtgaggcttaaaaatgagaaagtataagtaagttttcaagcacagaatcttacatacagtaggcggtaaatatatagtagttatttttataattatttcaaaagagaatagagagaaaccaaattagaaaagtaggttggggttgtgaaaggaaaatagatttactgagcatgagatgaatacataactgaatatttccttaccttccccctttctcttgcaatgtgtgaattaccatgccctcccactatcctctccagctcacttttctcctataaatgttgaagccctcaaaattatttttggagaaaggcacaaaccacagactgtttctataaattctgtgttcttttcttctgggcatgttcttaaccttggcaaaataaacttgtgaattgattgagacctatctcaaatagtttttggtttacagggtcatattggtgtataaaaggtggctgagtttattcttgagcaatgaggaggcctcgcaggatattggagggggtggtgacatgatccaagtggtatgaaatacatttaggaatctactgatgtatgacccctacacccatctgaatgaaaatgctcttgctaagttaaaaaggaccaccatctcattgccaaatgcagttcttatcccactgggctatagcagcattagacaagatttacaagttgtctttcaaatgctttttttttttaaccgacttccacattgtcattcttttccagatttccttgacctctctgattgtactttctccgtctcccctgaataatcttcctctccccactccttaggtacttggaccttctaaaggtttatctttggtcctcttttcttgcattctattcctgttccatggcccatcatatctatttcccaacttacatgctaatgattcccaagtttcaatttgcagccccttctactatttctctatcctggcaataccaaacagcgtgcagtgtcctgaatgccacatttccacccccctgcccctaagcctttctcattgagcactagaacgtcaacttccatgtcatctaccgggcaaactcctgcacattgattaaggtccaaattgctcattgtatcctcagtcatgcccttcttggtgcttctagggaactgtgcctacattcactcatttttacgctcaaggctgtgtgtactgatcacttatagagtacttgcctcagagttcttgtagcggagaactctgcttatctgtcttcctgttagtctatgagcaagagaacaggattcagctgtgcccattgctttatattgagcaactagtattgtctctaggacataagaggtgggtactcaagattcactggtgaataaatcttggaaagatgattcatgctaccaaaaccccatacaactccactgtaatggttaaatgaaagtcagtgagattcattttcatcttgtccattttcatgcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatggtatggacattttctcatggcttgttttggaggttctttaatctgatatgggaaaaggtatttattatgtaggaaatatttactttttgccacataaggtatgaggtatttatgggtaactggaaattactactggtgaaaaaatttaagtatatatcagaacatgtcaacttttggttttgtattattgaaaagggaataagagaggtaactgaatgaatggacttgggatcctgtgaaatatctaacagaagtttttttcttaaagatgatgaaacagtctcacagaactgaaatggcttgtccaattagtgtcagcattctaactagacccctctggttctaggtaaaaacttcccaaaactttagagttggaatatattctaaaaaccattttatagaggagtaaactgaagacaagagagggaaagtagcccgaagtcaaaacgcaatgtagtggcggaaccagaacaagagccctaatctaaagtcttttccataatattatgcaaagcaaagtgtagctttaaattttggaattcttaaagaacaggtgtttaatatcaagattagaagggattgaaacattttgttcatcctgctgaagcgtgaatttcccctgtaacatccccttagatctgtgctatccttcaacatttctgcctgaggcagcccatttccttttagatgccttggacttgatggaacatgacaaatgttcaggagatccaatttcttcagtgaggatgccatggcttttgattcttcctcataaatggaggacaaatagagcctaataccttcttctcttctgtatattctctcccctatttttagttatttgtgacataaagatcagctcctctgtctggagcatttggtcgatgaggtcaaggtcatgagctccatccttgagtagacaattcactttgccttgatctctggttccagatacttggtgatcccacaagggcctgggagtgagccagtgtaaagcctatcatcatgtttgaaatagttactgtaagcacagtcctagtggattacagaccagtagcagcatcctcccatgaagacagtaacaaacaaaggtagttaacagtaaccatacctgcttcctaattttaaaatatcattaattcaagtcaatgaacagatattttattatttttaaatattccctgacagcaggtaaaaatggttgatgactatgtttcttaagatgatattagatcagtgatgaaataatctgtacaacaaaccccatgacacgagtttacctatataataaacctggacacatacccctaaacctaaaataaaagctttttttgtttttttttataaaaaagatagtattagacatggttaaaccaccggtttggaaaactcctgctttttttcttgactctgcccctgatttgtgaataatgttaaataaattcctttattccttccattcctccctccctctttctctttctttcttcttccaaacatatttatcgagcacatactgtgtgcaagacatttttctagacactggagacacagtgatgtgaaaagtagataacatcccctgcttacatttcaatgcatttccttcatacactagagatgaactccactgcctctttatgtcactaataaaggataaaatgaaataacatgatttgttaaagcattttaaattcttggtaaggaagatatccttacatttaaagtacattgtacttttcaccatccagtggatagtgccctccaggagggctcttttgtttcatttcccctccctggtttacaggcttttcactgaatctgtttagtatttgtttatttaacaaagactcacagaaggcatcacgtagcagacatgggactaagttatacagtcttaaatgagacattaacagtcactgacttcatggagctttccagaataaacagtgtgactgggaaagtggatgaaatagctctaatgaactctgataagaggtatttaagggaggaaactgaaactaatgaagaaaatgaggattagaagtaactttgggttttatttacctgttctgttccagggcttcaacctactccaaatcccttagctgagaaaataaatatatacatatcattatgaatactgtcaatttttctcatgtatccttatagtaagtacatcttgaggaataatattttgaggttaggaaaattctctttaacacaaaacatccactgtcatcttcatcgtaatatttatccttttctattttactttcagaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagtaagtcaatgaatatgcaatcagtaaaatgttttctaatgagtttgctgtgtatttaaaagcatttgactggcgtgtacacacacacacagaggcacacattaactaactactttttgtatttttagtttgttatattttatattttagtctccatttggataccatctcctcagataaaatttccttgggtcaggtgacactacttcatactctatattcttaagtattggcttactcattaaactatgatattatgttgaaagtgatcaatttattcattttacatattcttttatccagccttttaaacaaaacggtgaccccacattttaggcaaagcactggggatccaatgttaataatttcaggtatggtcctcaaccccaaaaaacgtttagtttatggcaggatgtggaaaattaaaccactgcaatttgtagtgttgtgatgggagggtacagggtccgttagcgtccaggggagatccaaagcccaactgggggtgtcagatgaggagaggcatttaagccctgagaaaggtgtgggtgtagaggggaacacagagaattccaagctagagagaacctgacacatttgggcaacagtgaccatttccatggttgcagcactacgttacttattgctgtgtcacaagtgcctcacactgtgcctgtcacatgcaagtatgaaataaataggtttttggaggaaggaattataagagaaaaagaaaaagaaggaaaaaaagaaaggagagagggagggagagaatggaaacaagaaagaaatctatcttctttttaccatcatagaccctggtcaattatttctttctttatttttcttatattttctttttgaagtgaagaactcatgtgtaacaagcagttccacatgcacaggtttaaaacagtacacttcttctgtcaagattctctctgttaacataaatcgtacttgggaggaaagacgcagcacctataagaactgttgccagtgtttaaatattactgaagttatgtctacaatgatcctgcaattttaaaggaaaaatgtattccatttaatcaaagccaaatacatgattttgctacctttttttctggttggattaatcttttgacccctcttagcttattttaaaggcacatggaatcagatccttagagtgattcattaatgcatcaattgttcctttgttcagtaaatataaattaattctttctgtagtgtcaggcaccatgtttgacactggaattagagagaagaccaagacacagccccaccctctaggagctcaagtctctagggcaggaagttactatagttgaataactacaaaacggggagataaatggcaaaggaggcacaactagagaaggctaaaggagattgaggggttaattctgccagaaggtggatgggatcagggccaggcttcacagaggagggaacacttgagctaggtaggcgtggagaataagtaagatttcaattttattaaattctctctgccaggagtacatgaagtgtggtgcctgagaggaattattagggctttcactgtacatttttggccatgcaatgcactttatgaatgttattaaattagtcttttgaagacatagctattttccaagggtatattctggctgaattaattaaaactaagagataactacataatgagctttactgtctgattttttaaaaatttttatttattttttatttttgtaggtgatttttttttacattgtttctatttcacccaacaaccatttctaatgtaaataccattgattggctggacacggtggcacctgtaattccagcattttgggaggccgaggtgggcagatcacttgaggccaggagttcgagaccagcctggccaacatggtgaaaccctgtctctattaaaaatacacaaattagttagatgtggtggtgcacacctataaaccaagctacttgggaggctgagatgggagaatcgcttgaacctgggaggcggaggttgcagtgagccaagatcgtgccactgcactacagcctgggtgacggagtgagaccctgtctcaaaaaaaaaaaaaaaaaatttgattgatgaaactgcactagttatgcccacctgcttgtgatgcttgtgatgggtgatccacagctaatgtattgttttcttttctccccaaaggcgagagatagttggggtggtggaacctgtgccccatgatgaaacatactgtgaccccgcatctctgttccatgtttctaatgattactcattcattcggtaaattacagttttcttgtttctgtttaacttctagggtttgtggacagctgtgctaataacgacaaaaagtagggataaatgaagtgataattataatttttctatttccacccataactaccaagctcaatacagatgctccttgctttacaatggggttacatcccaataaactcatcataagttgaaaataccttaagaatgcgctgaatacacctaacctatggtacaccaaagcatagcctagtctaccctaaacatgctcagaacactcacattagcctactgttgggcaaaatcatctaacacaaaccttattttataataaagtgttcaatatctcatgtaattatttaatactgtgctgaaagtgaaaaactttgggtacttaccattaacatatacagctgaaagcgctattgtaaagtcgaaaaatcaaaagtccaaccgttgtaagttgggaactgtctgtatagatgatcaatttcaattcaatttaaactaacacaatttattgggttaaattaaacttgtgtaagatcttgtccccgtcatcagcaatcaatagtatcattgggagaaataaaaagtagttttgtcttcttgttactggcagtttattgtacattgtgatagagaatttttaacttgaagtccaaaaacatatgttcttcacctagtaaccccagtccttgaatttgctggagctcagtttatttgtaaaatgaaaataatattatcgacctgttataaggatggatttgcatcatttatgtgaaagggctattaatctgtaaaacacaaaacaaatgttagctaacatttaacagagtaatattgccatgtttatcaacagatacataaaacacaaataggttatgatgagtgtagagttcatgtctggagaaagagatttcagagatttgatcagtatctctttggttacttgggctccagatttaaatatatgccctaatctataactctaagacagtaagtaaacacgggactgcttttttgttgctttgtctcctgtgcagatattacacaaggaccctttaccaattccagtttcaagaagcactttgtcaagcagctaaacatgaaggccctctgcacaaatgtgacatctcaaactctacagaagctggacagaaactgttgtaagaaatacctcaaaatgttgaacctctcctagtattcagtattactcatttccatgcctaggtttgtatttgatttctttgttctaaaaagaaaattttatggcctcaaaatgtcctcatttacaaaccaaacatttaatttgtggtcagacaggaacctagaccatacaacaattgggtgggccacctcttttctccctatcataactacagccctctcttcctggtaattggaaggaaagagcggtttagggtggaatatatctgttaatatgcattcttttcttatctgccagaagcaaatttagccaagtcaaagagaagaaaccatagatcatagatgtaaatatatgtacatctggaacccctcaaaaggccctgaaccccctttttttgtgtagcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatgtgagtacacccagttgacaagtttcacacccattccttgtctacttcgtctagtctcctttggccccagtctcatgaggacttgtgacacagctgagaatgtttctttctctatagtacctgcttctttctctgctatggtgacagctgccattttatgggagaaaaatcacaactgttgagcacctatgtatgagacatagtgaaagcaactgtacagacaaacacacacagatgcatgcacatgtgtgcatacacatacaagagtgtgtactctttcaagtctgttttattagtctcatctttaaaatacatattataactgtctccaaccaactgtactcgaatcctgctttccaaagacaaatactttttttaaaacaattttaatgctattatctcttctttctggcagtcactttcatatttctaaataattttcttatattgctattttgttattgctcagttttaaaccttcgttttggtctactcttccacttccatttcttgctcttccaataaggccttataatgatttttagtaaaacagttatcaatgtttacatcactatgcctgaagagccaagtaatgtattattttgtgtcctttcttatgtggcttgttgtttgtctcaaagtttctacttgcctgaatttttcggcttacctaatttgctatctacgttgtatttgtttttctcccaaatatttcaacatatctattcttttcagtctcattttttttccctagagacttccctcttggagtcttatattctcctttcccaacatttgttgttctcctgggttgaatccactgcttcctagattccatgtcttctttcttggattacctcttgtttttctggagcatagcctcccacagactgtccaagcaggggcacaaggggtaggaggatcaactgttctccaaagtggttttaaccacaccctgacttccattcctcttcttttcagcccagtattattttcccaccctcacctgtgcttgaattccaaacctcttttggattttttggctctttgtgggtctgtgcctcctctgtgtacaaattaggtcatggcttcctgtccctgagaaagcagttacccctgtctcatcatttctcgttttccaaaagcctgttgatacctttaaaagatccctttactatcactttagtgggatctttggaggaaaaggaaataagcacatgtgggcaatctgttgtctgtaaccagcagtctgctatttctctttaatcagatgcagaccaaagcatcaaagtgaggataagcctaaaatcagctcttggagataaagcagtgagtattctggacagtgaattgattatttttgagtgcacagtcttcacttaaataggacacaataaacccatttcagtgtgaactgaaagaagagaggccatgtggttcttttcaaaactataagttccagattctggcctttggctttgggtttttaaagtaacctattccttattggataagctattcctacttcttattgccttgaaggtgttatgaacttcaatgagaaaatatctgtatcaatccttaaatatcagataccaatcctttcaattgactctcgtcaccattacttttattctagaaagagaatatggttatattttgaaaggtagtttgtttttagaagcggtacagttaggtttttttagtttgttttgttttagtcaatcacctgtatcctggcttcactcagagtgtggcccatggtccagcagcatttgaatcacctggaagcttgttaaaaatgcgtaatttcaagcctagccctggaccacttaatcagaatctgcattttagcaagcctccaggtgatttgtacgtacaatcaagactgggaagctctaatttagaacactgcttctcaaacttggctgcacagtggaatcacatgggaagtttaaaaaatattaatgcctaggttatacacctccagagattctgatttaattgttctggggtatggagtcaagagttgggcatcaagagtgtttaaagctccccaggtgattctagtcacagcaaagtttgagaaccacttatttagggaaatgcattttgggggtaacaaaatattttacaggaaattatgttatttacaccttaatgtgaattatttacttagctattgctctgtcgttaacacctgtctcagatgatccagtctgaggaagtggtagctttgattgggaaagaggagaggaaagagtagtctcgggtagatggggctcaaaagcctgagagtgggtatgacaccctgaaggctttgcagggttagaagtcaatctgggactattggataaaatggtccaagatgctacagcaaattgttgtctgcattggatagatccaaagattgtgtttctacccccttacctttcctgaaaagttaaacattgtccaagattcaataacacttttagtataattttcatagttaagacatcaaaacataaataactcaggagtaaaaagatgaacattcaagaaagaaaaaatgaaaatggaagtgcatttctttccaatcattaagagttcaagttcctatttcataataatcattgggtgattgtgggactatttctctgtcatatttttattagctcttccttggcttaatacattttaattagtctcggcagatcaggatattttcaattatcttttttcttgacatttatactcatcctctttccatgcagtgagggttggtaaatagtgttcagggggtttgattctgtaatgttctgaggccaattcactgagcaacaatctgttttgatgaaacgagatcatatatttagtgcatctacacaacagggtcagaatatctgctcagttcaaactatttcatggtgaattccaatggatctatggcattattttgagaactcagcctttaaaatcttttatattttatatttatagagatagtagccagactgttattagtaacaatattaataatattattactattaaaggaactcctctaaacctcggtgaaaccaagctgaagtgaatgtaacaattcttttgtgaaatattataattttttaaagggaaaggagaaacatacaactagtgccattcattgattcctactgccagctactggaggaaagagtgaatgctttcttttaagtctttaaaagctatgcaaacataagctatttgtttagcactggaaaacaagaacaaacagattagcttgctgtttacaaagtgttatttttcatttgaatgtcaagtttttcttttacacttatagataagtacatttctgagaacgtaccataaaaatagtaaaaactgattttgtccacaaggtacagacattttgctacaaaacccacaacttttgtttgactcccatcggtaaacctacactcaaggtgttaatttcagtttacatcaaactaaagtaaaaggacccgtgaacccaatggttcctaaagcatggttgcctgaccagcagcattaccaccacctgggagtttgttagagatgtaaattattgggtttcatctcgggcttcctgaatcagacgctctaggggtggacccagcactcagtgttgtaactaacccttcaggtgatacggatgcacacttacattgaaggtcactgacttaatgaatagcaagaatctctggaatattaagaataattcttgaggagtatcagattataaatgtgtcttacgagtccctctgagcagtgtctttcttctgaatttgcagtatgaatggaacgacaatgaaatgtacctgttccgatcatctgttgcatatgctatgaggcagtactttttaaaagtaaaaaatcagatgattctttttgggtgagttgatttgctgggttctcaaattactccaactaggatttctcttactcttgagtctgaggagatactctgcctaaacttttcttcaagtgaaattgataaaaatctcctagtaacttatacttctttggtattgtggaatttgggtgactccatagagggctgatcgtgccttatttgactggtaaattgcaagagtattgttcttactaaacagctgtcactctcttctcagaatgctctcatacaccctcagcactgaaagaaaaaaaaggcagaacttcctgccttcattgacctatattctagtatgtatgttgtgtatgtgtctatgtgtgtaatatgtaagtgaaatatagagttacaaggtactaagtgctaggagaaaaatataaaagggtctggggagggctggtgtgtgtgtgtgtgtgttgggcagggggtaggtcaattttaaccaaaatggtcagaaaagacgtcactaatacttgaacaagtatatgaagcaggagagagggagagagagagcgagagagcgagagagcgagagcgagagccagcaatgcagacaatggggaaagtgtctcagctgggggaaacagcaggtgcagaccctgaggaaggaatatcctcagcctgttaaaggaacagcaaggaggccagtgtggctggagcagattagaagacaagtggagacatggccaggacgatgttagggcatagaggaaggagggagagacaaaaggtcatgtgggccttgaaggccattctaaggactttggcttttattctgagttagatgagtagtcactcaagggctatgagccacatgatctaccttattttgtgctaggatcgcttgacagctgtgagagcacagtcctgtcacatcatatggtagaccccactcattaccttcatccatgggtgcaacacgacgttttctgctgggtaggaccaggttgtgggtgtaaatccgtgtgtggtactcagccatttctgcccaccacattgtttgccacaatcctgggccaccgattattccttgttgattctcctgctgttttcagtttggaaattttctataattgaaaaagatgtgggttttatataaaggcagaacaaatagtgccaaaacttgatatcacaatttttatgtagttgatttcatgtgctttgggcagcattaatgttttttttgacaattacatcctctcattgtttgccctcccccatgtctctctatcctgtgattctctctctaatctccttttcaccagcaggggaagtcttcattctcttgattgtgtcctctgtgccacaagtgaagatgtttgttttgtttctctacagggaggaggatgtgcgagtggctaatttgaaaccaagaatctcctttaatttctttgtcactgcacctaaaaatgtgtctgatatcattcctagaactgaagttgaaaaggccatcaggtgacattttactttcatctaagggtgagggggttaccaaatacaacaacaataaccagtattttgtgtgttcttcctgtgtgccaagcattattctgagtcatttacatgtgttacctcatgctacaaactccacgtaattccagaaggatcccacagcaggggaataatcttgatttgagttccgccagatgtaggctcttacaaaagggaagaaaaactatagattacatgttatcaagcaagttaccactatgggcaactagagctcagtcctgctgggaaatgtggagagactgtgtagaatgtgcacctgaattgtcccatcagagggctgaggaagctggggtatttatttaccacctcccatcagttattggctgagaactgcttccaggtggcattaattccccagcatttcagccctgcaggcagggcagatcttgtggtctgagaatgctctctggtaaaggggagcaggtgctggttgaagaaagtctggctagaactcatggaaaatagtaagtgcagaagggacgtggacaggtattgccatcatttgctctcaggactttgatgtaggattcatttaactgcccaaatcctgaaacaaggtaggcaagtacaggattccttacatattgcttggcctctatgctaggtgagggacttgcaatgaatgtttagaagaaacagtttgtccaatttgcctcaattgtttcccacaacagacagcaggagcgaagaatggattgaccttgcagatggtttcctgtcattataaaaaggcttaatatacctactgcttctgggcagctcactaggtggccctctcgagctgcgtcagagtcctgtttaacttcggaagcagatctaagacctgctgggcagggtgtgcctagaaaatctggcttggttggagtgtatgacccaactgtgcccagagactcactcacttcctccagaagacttgtgtctgtcttgggaaaaacttctggctgagatctttatgaaaaagctcagccttcctggatactcagctgcaagctacaggggacctcagactctctgtttccaggatgactcttttcagatcttttctggctgaggttgccaggggttacctgactaattctgcgttgatctctctctctctctctggtggactctgacctcctgacctgggctatacccaagggtaggaagatgagatgccagacactttggtctttgatccagaatgttctccatatcattgtttttttttttctttaaacaatctttatccttttgcctccataaaagtaagccatttcccatcccagtgctgaagaaactggcaaaggtccctttcttatgtgcctccccagtgctacctccaaatgccaataccttttatttggaaaatactactatagagacttggtcataggacctgattcatttgtataatagaagaggtgtatccaattatccgtttccattatttgatataaagtcattgtagatgtactctgacagaagggaaattcttgccaaatatgataactttgcccttaaacacagcagtcacaaatgaataaatgccaaccatttatacatttccacacttacaactcaattttccaatggagctgttgatgaacctaatctaggttgcaaggcatgaaagatgcataattgtcaaagacttatatctttaattagacctatttactttgctcttatgattttaggtgtatattccttttttttttttttgaaacagagtcttgctctgtcacctaggctagagggcagtggcacaatctcggctcactgcaacctctgcctcccgggttcaagcgattctcctgcctcagcctcccaagtagctgggactacagacatgcgccaccatgcctggctaggtgtatattcttatgataagctctattatatcctttcaggaacaatgatataaacagtaaataactacacacaattttattttgtctaagtgtcccctttgctgttttttgcttttgcaaataggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagtaagtggcctttcctagacttaactatccaaaaatagaaagatttagagaacagtaactggaaaattttcagtaagtggtagattgtatagaccaattacttagttggacaattttgcaactaaatactctgatgcattccctagaaatttaatagatcagcagttcatcttcctagtaagcccacacagtaagtatctggcctgggatttattagcatgccctagtaaactccatcccagccacagtgtcatttccctcctacaaagaggcactgcaaactcagcaggtatgggtgggctcagggaatggttttcactgctctcactgctgaatccttttcagtctaaaagtggggactggattgctggagactggattgctgatgggtttgaaccgtgaaattgttcaagaatgggctgtgagatttctttgaaactgctactcttggttttttgtttgtttgtttgtttgtttgtttgagacagagtcttgctctgtcacccaggctggagtgcagtggtgcaaccatagttcactgcaacctcgatttcccaggctcaagtgatcgtcccacctcagcttcctgagtagctgggactacaggggtgtgccaccacacccagctaatttttgtattttttgtggagacagggttttgtcatgttgcccaagcttttcttgaactcctgagttcaagcaatccgcctgcctcgccctccagtgctgggattacaggcgtgagccaccgtgccctgcccaaactgctactctttactcagctttatttctcaccgggtagctcagactctatgtcattctcttagttcatggttaggcacgttctaatttgttcctgaactgaagatcaaagttcattgaggatttaaattaccagagacctgatttctagtcttgctttgctactagatgtttttcttctgtccttaagaaattgactcttgctgggcttatggaatgaaggttaatattttatagcacctaacaaaactctatgcacacctcagcttgtttaaacatattcttgattgaatctctatatttgctagtctattttgctcaattttcttcactatagagatttttcgtttctccaaaatagctccatctaaaatattaggcattcaatgtgtaatacattcatatggttaaaattaaaaaatacaaaggtgaacatgaagagtcttccttattaccttttgcctccaagttatcttcccaaagccaaacaaaattatcagttccttggagatacacacacacacacacacacacacacacacacacacacacagtcttttttcttttttctcagacatatatatatatatatatatatatatatatctcctttttagtatacaaatatatacttttttagtgtgtatatttgtgtgtatgtgcatatgtatatctcatttcttagtatacaaatgttagcaggctataaacaatgttctgcatattttgttttccacttaatggtttttttaaatttttatttatataggtgtgatccaccacacccagcccacttaatgtgttttagagatcattacacattagttgcacaaaaaacttctccttttttctggcagtataatttcccatttcacgaatggattgtaattttgataaacagtcatctattgatgaacttttatgttgtttccaaacatttgtgtttacttaaaaaaagtgatggcttaatataggtaatttcctgagtatgccaatatacccttaagatgaatcctagcagtgggaaagctgggtgaaagcatacgtgcaattgctaattttgataaatattgccaaattgccctcaatagcggctgtaccaatttatactcacatcaacagtttatgaaacttcctgttcctccctcccagcctccttaacacagattcccctgaaactttttgattttcatcagtccgattgattttaacataagtatattaaggcaaccttgctctatttaacaggaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgtatttgctcacagtgtttgagcagtgctgagcacaaagcagacactcaataaatgctagatttacacactc

SEQ ID NO: 3 - DNA, 17 bp, Artificial Sequence
  antisense oligonucleotide
gaagagcttgacatcgt

SEQ ID NO: 4 - DNA, 17 bp, Artificial Sequence
  antisense oligonucleotide
cttcggcttcgtggtta

SEQ ID NO: 5 - DNA, 17 bp, Artificial Sequence
  antisense oligonucleotide
acaggtcttcggcttcg

SEQ ID NO: 6 - DNA, 17 bp, Artificial Sequence
  antisense oligonucleotide
gcattcttgtggattat

SEQ ID NO: 7 - DNA, 17 bp, Artificial Sequence
  antisense oligonucleotide
gcctctcattgtagtct

SEQ ID NO: 8 - DNA, 17 bp, Artificial Sequence
  antisense oligonucleotide
tgccgacctcagatctc

SEQ ID NO: 9 - DNA, 17 bp, Artificial Sequence
  antisense oligonucleotide
caactggccgcggctgt

SEQ ID NO: 10 - DNA, 17 bp, Artificial Sequence
  antisense oligonucleotide
tcaatcaactggccgcg

SEQ ID NO: 11 - DNA, 17 bp, Artificial Sequence
  antisense oligonucleotide
gaggcatccaattggac

SEQ ID NO: 12 - DNA, 17 bp, Artificial Sequence
  antisense oligonucleotide
atacaaagaacttctcg

SEQ ID NO: 13 - DNA, 17 bp, Artificial Sequence
  antisense oligonucleotide
tcgtccattgtcacctt

SEQ ID NO: 14 - DNA, 17 bp, Artificial Sequence
  antisense oligonucleotide
aggaagtcgtccattgt

SEQ ID NO: 15 - DNA, 17 bp, Artificial Sequence
  antisense oligonucleotide
ggttgtgcagcatatgc

SEQ ID NO: 16 - DNA, 17 bp, Artificial Sequence
  antisense oligonucleotide
aatcgtgagtgcttgtt

SEQ ID NO: 17 - DNA, 17 bp, Artificial Sequence
  antisense oligonucleotide
ccttcatgtttagctgc

SEQ ID NO: 18 - DNA, 17 bp, Artificial Sequence
  antisense oligonucleotide
gagcagtggccttacat

SEQ ID NO: 19 - DNA, 17 bp, Artificial Sequence
  antisense oligonucleotide
ctccagtcggtactcca

SEQ ID NO: 20 - DNA, 17 bp, Artificial Sequence
  antisense oligonucleotide
tcggaacaggtacattt

SEQ ID NO: 21 - DNA, 17 bp, Artificial Sequence
  antisense oligonucleotide
gatcggaacaggtacat

SEQ ID NO: 22 - DNA, 17 bp, Artificial Sequence
  antisense oligonucleotide
tcaaattagccactcgc

SEQ ID NO: 23 - DNA, 17 bp, Artificial Sequence
  antisense oligonucleotide
catcattgatacggctc

SEQ ID NO: 24 - DNA, 17 bp, Artificial Sequence
  antisense oligonucleotide
gttgtcattcagacgga

SEQ ID NO: 25 - DNA, 17 bp, Artificial Sequence
  antisense oligonucleotide
taggaggtccaagtgtt

SEQ ID NO: 26 - DNA, 17 bp, Artificial Sequence
  antisense oligonucleotide
tcgatggaggcataagg

SEQ ID NO: 27 - DNA, 17 bp, Artificial Sequence
  antisense oligonucleotide
atatcgatggaggcata

SEQ ID NO: 28 - DNA, 17 bp, Artificial Sequence
  antisense oligonucleotide
cctttgctaatatcgat

SEQ ID NO: 29 - DNA, 17 bp, Artificial Sequence
  antisense oligonucleotide
tcgattccaaacatcac

SEQ ID NO: 30 - DNA, 17 bp, Artificial Sequence
  antisense oligonucleotide
tagacctgtcaccttga

SEQ ID NO: 31 - DNA, 17 bp, Artificial Sequence
  antisense oligonucleotide
gttgatcaagcaccttg

SEQ ID NO: 32 - DNA, 17 bp, Artificial Sequence
  antisense oligonucleotide
atgagtttctatcaggc

SEQ ID NO: 33 - DNA, 17 bp, Artificial Sequence
  antisense oligonucleotide
gagtgtgtaaatctagc

SEQ ID NO: 34 - DNA, 17 bp, Artificial Sequence
  antisense oligonucleotide
tcttaccgcttaatgga

SEQ ID NO: 35 - DNA, 17 bp, Artificial Sequence
  antisense oligonucleotide
ttaggtgaagcgttcct

SEQ ID NO: 36 - DNA, 17 bp, Artificial Sequence
  antisense oligonucleotide
aatgatgcgagaggatc

SEQ ID NO: 37 - DNA, 17 bp, Artificial Sequence
  antisense oligonucleotide
taagtgtggtaggctag

SEQ ID NO: 38 - DNA, 17 bp, Artificial Sequence
  antisense oligonucleotide
aagctctagagaccacg

SEQ ID NO: 39 - DNA, 17 bp, Artificial Sequence
  antisense oligonucleotide
cgacttgtacctgtgtg

SEQ ID NO: 40 - DNA, 17 bp, Artificial Sequence
  antisense oligonucleotide
agctttggaactagttt

SEQ ID NO: 41 - DNA, 17 bp, Artificial Sequence
  antisense oligonucleotide
ggtcctatcaaccagat

SEQ ID NO: 42 - DNA, 16 bp, Artificial Sequence
  antisense oligonucleotide
aggattcgagtacagt

SEQ ID NO: 43 - DNA, 17 bp, Artificial Sequence
  antisense oligonucleotide
cacgttgtttgagataa

SEQ ID NO: 44 - DNA, 17 bp, Artificial Sequence
  antisense oligonucleotide
gtttacttccgaagcta

SEQ ID NO: 45 - DNA, 16 bp, Artificial Sequence
  antisense oligonucleotide
ctaactgtaccgcttc

SEQ ID NO: 46 - DNA, 17 bp, Artificial Sequence
  antisense oligonucleotide
gtaatgagccacacaag

SEQ ID NO: 47 - DNA, 17 bp, Artificial Sequence
  antisense oligonucleotide
ttgactacgcatgtgac

SEQ ID NO: 48 - DNA, 17 bp, Artificial Sequence
  antisense oligonucleotide
tgataacacacacggat

SEQ ID NO: 49 - DNA, 17 bp, Artificial Sequence
  antisense oligonucleotide
attaaagacggcctctg

SEQ ID NO: 50 - DNA, 16 bp, Artificial Sequence
  antisense oligonucleotide
gatctgccgagactaa

SEQ ID NO: 51 - DNA, 17 bp, Artificial Sequence
  antisense oligonucleotide
tacatacagaaagcggc

SEQ ID NO: 52 - DNA, 17 bp, Artificial Sequence
  antisense oligonucleotide
agttgtgtaagtatcag

SEQ ID NO: 53 - DNA, 17 bp, Artificial Sequence
  antisense oligonucleotide
taggagtcagatgagta

SEQ ID NO: 54 - DNA, 17 bp, Artificial Sequence
  antisense oligonucleotide
aacgccaatggatgcat

SEQ ID NO: 55 - DNA, 17 bp, Artificial Sequence
  antisense oligonucleotide
cattaccatacaacgcc

SEQ ID NO: 56 - DNA, 17 bp, Artificial Sequence
  antisense oligonucleotide
aagtcgttgtccttaga

SEQ ID NO: 57 - DNA, 17 bp, Artificial Sequence
  antisense oligonucleotide
tcgatccaagcgtattt

SEQ ID NO: 58 - DNA, 17 bp, Artificial Sequence
  antisense oligonucleotide
cttacgacatgtaccac

SEQ ID NO: 59 - DNA, 17 bp, Artificial Sequence
  antisense oligonucleotide
gtctctcttacgacatg

SEQ ID NO: 60 - DNA, 17 bp, Artificial Sequence
  antisense oligonucleotide
gtaaagcacggtctgat

SEQ ID NO: 61 - DNA, 16 bp, Artificial Sequence
  antisense oligonucleotide
cgatccaagcgtattt

SEQ ID NO: 62 - DNA, 17 bp, Artificial Sequence
  antisense oligonucleotide
ccgactctgtgtatctg

SEQ ID NO: 63 - DNA, 17 bp, Artificial Sequence
  antisense oligonucleotide
gcctaacttgccgactt

SEQ ID NO: 64 - DNA, 17 bp, Artificial Sequence
  antisense oligonucleotide
tagctgccgtgacttag

SEQ ID NO: 65 - DNA, 16 bp, Artificial Sequence
  antisense oligonucleotide
actcgtaagacacatt

SEQ ID NO: 66 - DNA, 17 bp, Artificial Sequence
  antisense oligonucleotide
ctactccgctgaaggtc

SEQ ID NO: 67 - DNA, 17 bp, Artificial Sequence
  antisense oligonucleotide
tttagtcctctactccg

SEQ ID NO: 68 - DNA, 17 bp, Artificial Sequence
  antisense oligonucleotide
ttaagcctcactctagt

SEQ ID NO: 69 - DNA, 17 bp, Artificial Sequence
  antisense oligonucleotide
ttaccgcctactgtatg

SEQ ID NO: 70 - DNA, 17 bp, Artificial Sequence
  antisense oligonucleotide
caccttacctaggcata

SEQ ID NO: 71 - DNA, 17 bp, Artificial Sequence
  antisense oligonucleotide
atggacaccttacctag

SEQ ID NO: 72 - DNA, 17 bp, Artificial Sequence
  antisense oligonucleotide
gacaatgtggaagtcgg

SEQ ID NO: 73 - DNA, 17 bp, Artificial Sequence
  antisense oligonucleotide
gcacgctgtttggtatt

SEQ ID NO: 74 - DNA, 17 bp, Artificial Sequence
  antisense oligonucleotide
ttgacgttctagtgctc

SEQ ID NO: 75 - DNA, 17 bp, Artificial Sequence
  antisense oligonucleotide
gattgtctatatgcgaa

SEQ ID NO: 76 - DNA, 16 bp, Artificial Sequence
  antisense oligonucleotide
ctacatctggcggaac

SEQ ID NO: 77 - DNA, 17 bp, Artificial Sequence
  antisense oligonucleotide
tctccgctacaagaact

SEQ ID NO: 78 - DNA, 17 bp, Artificial Sequence
  antisense oligonucleotide
ctcttgttctggttccg

SEQ ID NO: 79 - DNA, 17 bp, Artificial Sequence
  antisense oligonucleotide
ggaccgtctatgccata

SEQ ID NO: 80 - DNA, 17 bp, Artificial Sequence
  antisense oligonucleotide
aagtgatgcggtagtat

SEQ ID NO: 81 - DNA, 17 bp, Artificial Sequence
  antisense oligonucleotide
tcatcgaccaaatgctc

SEQ ID NO: 82 - DNA, 17 bp, Artificial Sequence
  antisense oligonucleotide
actgatctaatatcatc

SEQ ID NO: 83 - DNA, 17 bp, Artificial Sequence
  antisense oligonucleotide
accatgtctaatactat

SEQ ID NO: 84 - DNA, 17 bp, Artificial Sequence
  antisense oligonucleotide
taggccttgtagttgag

SEQ ID NO: 85 - DNA, 17 bp, Artificial Sequence
  antisense oligonucleotide
tgtctgctacgtgatgc

SEQ ID NO: 86 - DNA, 17 bp, Artificial Sequence
  antisense oligonucleotide
tcagctcactattcagg

SEQ ID NO: 87 - DNA, 16 bp, Artificial Sequence
  antisense oligonucleotide
gaacgtgcctaaccat

SEQ ID NO: 88 - DNA, 17 bp, Artificial Sequence
  antisense oligonucleotide
cgtagtgctgcaaccat

SEQ ID NO: 89 - DNA, 17 bp, Artificial Sequence
  antisense oligonucleotide
gatctgattccatgtgc

SEQ ID NO: 90 - DNA, 17 bp, Artificial Sequence
  antisense oligonucleotide
gagacttgagctcctag

SEQ ID NO: 91 - DNA, 17 bp, Artificial Sequence
  antisense oligonucleotide
gcagcgcaattctgatc

SEQ ID NO: 92 - DNA, 17 bp, Artificial Sequence
  antisense oligonucleotide
gtttgaaggttcacgta

SEQ ID NO: 93 - DNA, 17 bp, Artificial Sequence
  antisense oligonucleotide
tgtgacaaggtaactca

SEQ ID NO: 94 - DNA, 17 bp, Artificial Sequence
  antisense oligonucleotide
cctatctccggctactt

SEQ ID NO: 95 - DNA, 17 bp, Artificial Sequence
  antisense oligonucleotide
aagagccaagtacacga

SEQ ID NO: 96 - DNA, 17 bp, Artificial Sequence
  antisense oligonucleotide
gagcttagccaatcaac

SEQ ID NO: 97 - DNA, 17 bp, Artificial Sequence
  antisense oligonucleotide
tcacttatacgattcca

SEQ ID NO: 98 - DNA, 17 bp, Artificial Sequence
  antisense oligonucleotide
cgaggtatggctgtgga

SEQ ID NO: 99 - DNA, 17 bp, Artificial Sequence
  antisense oligonucleotide
ttcgtgtacttaacttg

SEQ ID NO: 100 - DNA, 17 bp, Artificial Sequence
  antisense oligonucleotide
gaattgtgttcaaccgt

SEQ ID NO: 101 - DNA, 17 bp, Artificial Sequence
  antisense oligonucleotide
ggcctggtccaccattg

SEQ ID NO: 102 - DNA, 17 bp, Artificial Sequence
  antisense oligonucleotide
taaggatcctgaagtcg

SEQ ID NO: 103 - DNA, 17 bp, Artificial Sequence
  antisense oligonucleotide
actatctctcgcttcat

SEQ ID NO: 104 - DNA, 17 bp, Artificial Sequence
  antisense oligonucleotide
gtccttgtgtaatatcg

SEQ ID NO: 105 - DNA, 17 bp, Artificial Sequence
  control sequence
gatcattcgcggacaac

SEQ ID NO: 106 - DNA, 18 bp, Artificial Sequence
  control sequence
cgtttaggctatgtactt

SEQ ID NO: 107 - protein, 8 aa, Homo sapiens
DRVYIHPF

SEQ ID NO: 108 - protein, 7 aa, Homo sapiens
DRVYIHP

(19) United States
(12) Patent Application Publication
(10) Pub. No.: US 2024/0067973 A1
(43) Pub. Date: Feb. 29, 2024

(54) Oligonucleotide for reducing the expression of angiotensin-converting enzyme 2 (ACE2) and its use for treating viral infection

(71) Applicants: The First Affiliated Hospital of Sun Yat-sen University, Guangzhou Guangdong (CN); Secarna Pharmaceuticals GmbH & Co. KG, Marburg (DE)

(72) Inventors: Frank JASCHINSKI, Puchheim (DE); Anne SADEWASSER, Marburg (DE); Sven MICHEL, Bernried (DE); Marta Lucia DE LOS REYES, München (DE); Richard KLAR, München (DE); Nan ZHANG, Guangzhou (CN); Claus BACHERT, Guangzhou (CN); Weiping WEN, Guangzhou (CN)

(21) Appl. No.: 18/270,603

(22) PCT Filed: Dec. 31, 2021
(86) PCT No.: PCT/EP2021/087895

§ 371 Date: Jun. 30, 2023

(30) Foreign Application Priority Data
Dec. 31, 2020 (EP) 20217970.1
Dec. 24, 2021 (CN) 202111602866.6

(51) Int. Cl. C12N 15/113; A61P 31/14

(52) U.S. Cl. CPC C12N 15/1137; A61P 31/14; C12N 2310/11; C12N 2310/32

(57) ABSTRACT

The present invention refers to oligonucleotides comprising 10 to 25 nucleotides, wherein at least one of the nucleotides is modified, and the oligonucleotide hybridizes with mRNA of angiotensin-converting enzyme 2 (ACE2) of SEQ ID NO.1 and/or with pre-mRNA of ACE2 of SEQ ID NO.2 resulting in a reduction of the level of ACE2, ACE2 mRNA, ACE2 pre-mRNA or a combination thereof of 30 to 99% compared to an untreated control. The invention is further directed to a pharmaceutical composition comprising such oligonucleotide. The oligonucleotide and the pharmaceutical composition are used in a method of preventing and/or treating a viral disease.

The present invention refers to an oligonucleotide hybridizing with ACE2 of SEQ ID NO.1 and/or 2 to reduce the level of ACE2, ACE2 mRNA, ACE2 pre-mRNA or a combination thereof and a pharmaceutical composition comprising such oligonucleotide. The oligonucleotide and the pharmaceutical composition, respectively, is used in a method for preventing and/or treating a viral disease such as COVID-19.

### TECHNICAL BACKGROUND

Angiotensin-converting enzyme 2 (ACE2) is a zinc-containing metalloenzyme present on the surface of cells for example located in the lungs, the upper airways, arteries, heart, kidney, and intestines. The transmembrane protein ACE2 contains an N-terminal peptidase M2 domain and a C-terminal collectrin renal amino acid transporter domain. ACE2 is a single-pass type I membrane protein with its enzymatically active domain exposed on the cell surface.

The primary function of ACE2 is to act as a protease and to counterbalance the Angiotensin-converting enzyme (ACE). ACE cleaves angiotensin I hormone into the vasoconstricting angiotensin II. ACE2, in turn, cleaves the carboxyl-terminal amino acid phenylalanine from angiotensin II (Asp-Arg-Val-Tyr-Ile-His-Pro-Phe) and hydrolyses it into the vasodilator angiotensin (1-7), (H-Asp-Arg-Val-Tyr-Ile-His-Pro-OH). Thus, ACE2 is as an antagonist in the renin-angiotensin system (RAS), which is essentially responsible for human fluid balance and blood pressure regulation.

ACE2 can also cleave numerous peptides, including [des-Arg9]-bradykinin, apelin, neurotensin, dynorphin A, and ghrelin and regulates the membrane trafficking of the neutral amino acid transporter SLC6A19.

Moreover, ACE2 plays a prominent role in viral infection by viruses such as coronaviruses. The binding of the external spike S1 protein of coronaviruses to the enzymatic domain of ACE2 on the host cell surface results in endocytosis and translocation of both the virus and ACE2 into endosomes located within cells. Further, the host serine protease TMPRSS2 is also involved in this entry process priming of the viral spike S1 protein.

SARS-CoV-2 was first described in Wuhan, China, in 2019 (Huang et al. 2020, The Lancet, https://doi.org/10,1016/S0140-6736(20)30183-5) and has evolved into a pandemic virus in a relatively short time causing the Coronavirus Disease 2019 (COVID-19). SARS-CoV-2 may cause severe disease symptoms and has become a leading cause of death in 2020, especially in dense populated areas.

Therapeutic approaches, e.g., inhibiting the viral RNA polymerase using Remdesivir, exist and help to reduce the recovery time of patients with severe disease progression of SARS-CoV-2 infection (Beigel J. H. NEJM 2020). However, such therapeutic approaches do not contain or prevent the pandemic spread of the virus or lead to immunization of host subjects, but solely inhibit viral propagation in already infected subjects.

Extensive research is performed focusing on the investigation of vaccines against SARS-CoV-2. However, most of the vaccine candidates are currently still in test trials, even if some have already been or are in the phase of being admitted to the market. In any case the vaccines must first demonstrate that the induced immune response is sufficient to prevent infection with SARS-CoV-2, or to reduce the severity of disease progression in a broad range of the world population. Besides that, the production of sufficient vaccine amounts for vaccination the entire population will take several months to years after successful admission of a vaccine candidate. Furthermore, mutations of the virus may lead to sudden loss of efficacy of good vaccine candidates.

Hence, there is an urgent need for alternative therapeutic approaches for prevention and treatment of viral diseases such as coronavirus infections.

RNA interference (RNAi) may be considered which is a powerful biological process inherently applied by the host cells to destroy intracellular RNA viruses. However, siRNAs are absolutely dependent on delivery systems for activity. In vitro for example transfection reagents such as Lipofectamine are required. In vivo typically conjugations are used for cell-specific uptake of siRNA. Since the availability of cell-specific delivery systems is currently limited (currently only the GalNAc-modification for targeting hepatocytes is of clinical relevance), applicability of siRNAs is limited to these cell types.

Furthermore, the mechanism of action of RNAi takes place in the cytoplasm. Therefore only RNAs in the cytoplasm such as mRNAs can be targeted by siRNAs. In contrast thereto, single stranded ASOs do not require delivery systems such as transfection reagents or conjugations for activity in vitro or in vivo in many cells and organs. Naked ASOs are taken up by cells in sufficient quantities to achieve a sequence-specific target knockdown. In addition, the mechanism of RNase H dependent ASOs takes place in the nucleus, massively expanding the repertoire of RNAs or regions on RNAs that can be targeted.

Verma et al. (Front Mol Biosci., 2020; 7: 197) describe a combination of ASO and recombinant ACE2 protein for use in preventing and/or treating COVID-19, wherein the ASO targets highly conserved regions of the SARS-CoV-2 such as RNA-dependent RNA polymerase (RdRP), S protein and M protein.

Due to the prominent role of ACE2 in the infection of host cells by coronaviruses such as SARS-CoV or SARS-CoV2 the inhibition of ACE2 expression in host cells specifically mediated by antisense oligonucleotides (ASOs) represents a promising approach to reduce the risk of viral infection and/or to reduce viral load upon infection with a very low risk of undesired side effects.

Direct targeting of the virus with ASOs poses the risk that the virus could escape therapy due to mutations at the ASO binding site. It is therefore advantageous to target the host factor ACE2 which is not affected by viral mutations.

So far no ASOs exist which target ACE2 and are highly efficient in reduction and inhibition, respectively, of ACE2 expression via hybridization with ACE2 mRNA and/or pre-mRNA.

The ASO of the present invention are very successful in the inhibition of ACE2 expression representing an effective therapeutic approach for preventing and/or treating viral diseases such as coronavirus infections, e.g. SARS-CoV2.

### SUMMARY OF THE INVENTION

The present invention refers to an oligonucleotide comprising or consisting of 10 to 25 nucleotides, wherein at least one nucleotide is modified, hybridizing with mRNA of angiotensin-converting enzyme 2 (ACE2) of SEQ ID NO.1 and/or with pre-mRNA of ACE2 of SEQ ID NO.2 resulting in a reduction of the level of ACE2, ACE2 mRNA, ACE2 pre-mRNA or a combination thereof of 30 to 99% compared to an untreated control. The modification of the nucleotide is for example selected from the group consisting of a bridged nucleic acid such as LNA, ENA, a 2′Fluoro modified nucleotide, a 2 O -Methyl modified nucleotide, a 2 O-Methoxy modified nucleotide, a FANA and a combination thereof.

An oligonucleotide of the present invention is hybridizing with ACE2 of SEQ ID NO.1 and/or SEQ ID NO.2, wherein the oligonucleotide hybridizes preferably within a hybridizing active region of position 39345 to 40144, position 28945 to 29744, position 22545 to 23344, position 9745 to 10544, position 12945 to 13744, position 34545 to 35344, position 36145 to 36944, position 38545 to 39344, position 24945 to 25744, position 36145 to 36944, position 19345 to 20144, position 14545 to 15344, position 30545 to 31344, position 24145 to 24944, position 16945 to 17744, position 145 to 944, position 21745 to 22544, position 20145 to 20944, position 37745 to 38544, position 945 to 1744, position 18545 to 19344, position 5745 to 6544, position 11345 to 12144, position 32945 to 33744, position 8945 to 9744, position 3345 to 4144, position 26545 to 27344, position 28145 to 28944, position 25745 to 26544, position 6545 to 7344, position 15345 to 16144, position 4145 to 4944, position 8145 to 8944, position 31345 to 32144, position 2545 to 3344, position 7345 to 8144, position 12145 to 12944, position 20945 to 21744, position 13745 to 14544, position 4945 to 5744, or position 23345 to 24144.

Oligonucleotides of the present invention are for example shown in Table 1. The oligonucleotide inhibits for example the expression of ACE2, ACE2 mRNA, ACE2 pre-mRNA or a combination thereof at a nanomolar or micromolar concentration.

The present invention is further directed to a pharmaceutical composition comprising or consisting of an oligonucleotide of the present invention and a pharmaceutically acceptable carrier, excipient, dilutant, stimulant such as an adjuvant, or a combination thereof.

The pharmaceutical composition further comprises or consists of an active agent such as an antiviral active agent, an immune stimulating agent, disease specific agent or an agent that reverses infection-mediated immunosuppression or a combination thereof. The antiviral active agent, the immune stimulating agent, disease specific agent or the agent that reverses infection-mediated immunosuppression is for example selected from the group consisting of another oligonucleotide, an antibody, a small molecule, a lipid and/or a therapeutic such as a nucleoside analogue, a nucleotide analogue, a protease inhibitor, an ACE2 blocking peptide, an ACE2 fusion protein, a recombinant ACE2 such as Remdesivir, Umifenovir, Favipiravir, Chloroquine, Hydroxychloroquine, Dexamethasone, Lopinavir, Ritonavir, Darunavir, APN01, Favilavir, Molnupiravir, SNG001, Tocilizumab, Anakinra or a combination thereof.

The oligonucleotide or the pharmaceutical composition of the present invention is for example for use in a method of preventing and/or treating a viral disease. Moreover, the oligonucleotide or the pharmaceutical composition of the present invention is for example for use in combination with vaccination to prevent a viral disease. The viral disease is for example caused by a corona virus such as the severe acute respiratory syndrome coronavirus (SARS-CoV), the severe acute respiratory syndrome coronavirus 2 (SARS-CoV-2) or human corona virus NL63 (HCoV-NL63). The viral disease is for example Coronavirus Disease 2019 (COVID-19), Severe Acute Respiratory Syndrome (SARS) or Middle East Respiratory Syndrome (MERS).

The oligonucleotide or the pharmaceutical composition of the present invention is for example administered locally or systemically.

Furthermore, the present invention relates to a kit comprising an oligonucleotide or a pharmaceutical composition of the present invention and optionally technical instructions providing information on administration and/or dosage of the oligonucleotide or pharmaceutical composition.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

More specifically, all referenced documents are incorporated by reference to the same extent as if each individual document was specifically and individually indicated to be incorporated by reference.

### DESCRIPTION OF FIGURES

FIGS. 1A and 1B show the results of screening round of human ACE2-specific ASOs in HEK293T cells (FIGS. A.1 and A.2) and 1618-K cells (FIGS. B.1 and B.2).

FIG. 2 depicts dose-dependent ACE2 mRNA knockdown by selected ACE2-specific ASOs in human 1618-K cells after three days treatment.

FIG. 3 depicts residual ACE2 mRNA expression in ASO-treated HEK293T cells compared to mock-treated cells after 3 or 5 days treatment.

FIG. 4A shows protein expression in ASO-treated HEK293T cells analyzed by Western Blot after 3 and 5 days of treatment. FIG. 4B depicts the quantification of FIG. 4A indicating the residual ACE2 protein expression after 3 or 5 days ASO treatment in HEK293T cells.

FIG. 5 shows residual ACE2 mRNA expression in ASO-treated 1618-K cells compared to mock-treated cells after 3 or 5 days treatment.

FIG. 6A shows protein expression in ASO-treated 1618-K cells analyzed by Western Blot after 3 and 5 days treatment. FIG. 6B depicts the quantification of FIG. 6A indicating the residual ACE2 protein expression values after 3 or 5 days ASO treatment in 1618-K cells.

FIG. 7 depicts residual ACE2 mRNA expression in ASO-treated MucilAir™ compared to mock-treated MucilAir™ after 3 or 6 days treatment.

FIG. 8 shows the experimental scheme to test the protective effect of ACE2-specific ASOs of the present invention reducing SARS-CoV-2 infection in HEK-293T cells.

FIG. 9 depicts the protective effect of the selected ACE2-specific ASOs A43034H (SEQ ID NO.31), A43045Hi (SEQ ID NO.42) and A43081Hi (SEQ ID NO.78) in HEK-293T cells reducing SARS-CoV-2 infection.

FIG. 10 shows the experimental scheme to reduce ACE2 expression in human nasal epithelial cells (hNEC) after treatment with selected ACE2-specific ASOs.

FIG. 11A depicts the reduction of hACE2 protein expression in hNEC after 1 to 3 weeks of treatment with 1004 or 5 μAl of A43081Hi (2 replications) analyzed by Western Blot. ACE2 protein expression values are quantified by relative grey score. FIG. 11B shows the reduction of hACE2 protein expression in hNEC after 1 to 3 weeks of treatment with 5 μM of A43045Hi. ACE2 protein expression values are quantified by relative grey score.

FIG. 12A depicts the reduction of ACE2 protein expression after 3 weeks of treatment with 50 μM of A43034H (SEQ ID NO.31), A43045Hi (SEQ ID NO.42) and A43081Hi (SEQ ID NO.78) analyzed by Western Blot and ELISA. FIG. 12B depicts hACE2 protein expression after treatment of hNEC with 5 μM A43034H (SEQ ID NO.31), A43045Hi (SEQ ID NO.42) and A43081Hi (SEQ ID NO.78) and control oligonucleotides Neg1 (SEQ ID NO. 106) and R01011 (SEQ ID NO. 105).

FIG. 13 shows an experimental scheme for testing the protection of hNEC from SARS-CoV-2 infection by ACE2-specific ASOs of the present invention.

FIG. 14 shows the effect of 504 of A43034H (SEQ ID NO.31), A43045Hi (SEQ ID NO.42) and A43081Hi (SEQ ID NO.78) in reducing the infection of hNEC by SARS-CoV-2 variants D614G and B.1.617.2 (delta variant). The titer of SARS-CoV2 in apical medium is zero or close to zero that no graphs are visible.

FIG. 15 shows ACE2 mRNA of SEQ ID NO.1 (RefSeq ID NM_001371415).

FIG. 16 depicts ACE2 pre-mRNA of SEQ ID NO.2 (GRCh38: Chr X:15561033:15600960:-1).

### DETAILED DESCRIPTION

The present invention provides oligonucleotides which hybridize with mRNA and/or pre-mRNA sequences of the angiotensin-converting enzyme 2 (ACE2) for example of human origin. These oligonucleotides hybridize with an intron and/or an exon and/or an exon-exon junction and/or an exon-intron junction of the ACE2 pre-mRNA and/or ACE2 mRNA for example in a cell expressing ACE2 such as cells of the nasopharynx, bronchus, lung, salivary gland, esophagus, small intestine, duodenum, colon, rectum, gallbladder, pancreas, kidney, testis, epididymis, seminal vesicle, fallopian tube, vagina, ovary, placenta, thyroid gland, breast, arteries, heart, and adipose tissue, respectively. Via recruitment of RNase H the pre-mRNA is degraded and the levels of ACE2 mRNA are reduced. As a consequence the production of ACE2 protein is prevented and levels of ACE2 protein are reduced to the amount of ACE2 mRNA and ACE2 protein expression, respectively, for example on a cell expressing ACE2.

As a transmembrane protein, ACE2 serves as the main entry point into cells for some viruses such as coronaviruses, including HCoV-NL63, SARS-CoV (causing SARS), and SARS-CoV-2 (causing COVID-19) via the viral transmembrane spike (S) glycoprotein which is a trimer with three receptor-binding S1 subunit heads sitting on top of a trimeric membrane fusion stalk consisting of S2 subunits. More specifically, the binding of the S1 subunit of the spike protein of SARS-CoV and SARS-CoV-2 to the enzymatic domain of ACE2 on the surface of cells and the fusion of the viral and cellular membrane after proteolytic activation (e.g. by the cell surface protease TMPRSS2) of the S2 subunit of the spike protein results in endocytosis and translocation of both the virus and the enzyme into endosomes located within cells. In the cytoplasm of the infected cell replication of the virus is initiated and virus progenies are produced that are released from the infected cell which can then infect further host cells. In consequence, decreasing the level of ACE2 may result in decreasing the infection rate with a virus such as a coronavirus. Thus, the oligonucleotides of the present invention represent a promising and highly efficient tool for use in a method of preventing and/or treating viral diseases.

The oligonucleotides of the present invention hybridize for example with ACE2 mRNA of SEQ ID NO.1 (RefSeq ID NM_001371415) and/or ACE2 pre-mRNA of SEQ ID NO.2 (GRCh38: Chr X:15561033:15600960:-1).

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

Oligonucleotides of the present invention are for example antisense oligonucleotides consisting of or comprising 10 to 25 nucleotides, 10 to 15 nucleotides, 15 to 20 nucleotides, 12 to 18 nucleotides, or 14 to 17 nucleotides. The oligonucleotides for example consist of or comprise 10, 11, 12, 13, 14, 15, 16, 17, 18, 19, 20 or 25 nucleotides. The oligonucleotides of the present invention comprise at least one nucleotide which is modified. The modified nucleotide is for example a bridged nucleotide such as a locked nucleic acid (LNA, e.g., 2′,4′-LNA), cET, ENA, a 2′Fluoro modified nucleotide, a 2′O-Methyl modified nucleotide or combinations thereof. The oligonucleotide of the present invention comprises nucleotides having for example one or more, two or more, three or more or four or more of the same or different modifications. Further, the oligonucleotide of the present invention comprises optionally a modified phosphate backbone, wherein the phosphate is for example a phosphorothioate or methylphosphonate or a combination thereof.

Reducing according to the present invention includes inhibiting an effect such as expression in different percentages and amounts (levels), respectively.

The concept of the present invention is the provision of an oligonucleotide such as an antisense oligonucleotide mediating the limitation of available ACE2 mRNA for protein expression. In order to limit protein expression, the oligonucleotide requires the presence of a complementary nucleic acid sequence representing a hybridization target which allows the formation of heteroduplexes. The oligonucleotides of the present invention hybridize with mRNAs of SEQ ID NO.1 and/or pre-mRNAs of SEQ ID NO.2. The formation of a heteroduplex between the oligonucleotide and the target RNA leads to RNaseH-mediated degradation or inactivation of the target RNA and thus, limits the amount of available ACE2 mRNA for protein expression.

The oligonucleotide of the present invention comprises the one or more, two or more, three or more or four or more modified nucleotide(s) at the 3′- and/or 5′-end of the oligonucleotide and/or at any position within the oligonucleotide, wherein modified nucleotides follow in a row of 1, 2, 3, 4, 5, or 6 modified nucleotides, or a modified nucleotide is combined with one or more, two or more, three or more or four or more unmodified nucleotides. The following Table 1 presents embodiments of oligonucleotides comprising modified nucleotides for example LNA which are indicated by (+) and phosphorothioate (PTO) indicated by (*). The oligonucleotides consisting of or comprising the sequences of Table 1 may comprise any other modified nucleotide and any other combination of modified and unmodified nucleotides. Oligonucleotides of Table 1 hybridize with human ACE2 mRNA:

TABLE 1
List of human ACE2-specific ASOs.

| | | | | Position on |
|---|---|---|---|---|
| | | | | pre-mRNA |
| SEQ | | | | (GRCh38, Chr X, |
| ID | | Antisense Sequence | Antisense Sequence 5′-3′ with PTO (*) | 15561033_15600960; |
| NO. | Name | 5′-3′ | and LNA (+) | SEQ ID NO. 2) |
| 3 | A43001H | GAAGAGCTTGACATCGT | +G*+A*+A*G*A*G*C*T*T*G*A*C*A*T*+C*+G*+T | 47 |
| 4 | A43002H | CTTCGGCTTCGTGGTTA | +C*+T*+T*C*G*G*C*T*T*C*G*T*G*G*+T*+T*+A | 145 |
| 5 | A43003H | ACAGGTCTTCGGCTTCG | +A*+C*+A*G*G*T*C*T*T*C*G*G*C*T*+T*+C*+G | 151 |
| 6 | A43005H | GCATTCTTGTGGATTAT | +G*+C*+A*+T*+T*C*T*T*G*T*G*G*A*T*+T*+A*+T | 8700 |
| 7 | A43006H | GCCTCTCATTGTAGTCT | +G*+C*+C*T*C*T*C*A*T*T*G*T*A*G*+T*+C*+T | 9133 |
| 8 | A43007H | TGCCGACCTCAGATCTC | +T*+G*+C*C*G*A*C*C*T*C*A*G*A*T*+C*+T*+C | 9169 |
| 9 | A43008H | CAACTGGCCGCGGCTGT | +C*+A*+A*C*T*G*G*C*C*G*C*G*G*C*T*G*+T | 11571 |
| 10 | A43009H | TCAATCAACTGGCCGCG | +T*+C*+A*A*T*C*A*A*C*T*G*G*C*C*+G*+C*+G | 11576 |
| 11 | A43010H | GAGGCATCCAATTGGAC | +G*+A*+G*G*C*A*T*C*C*A*A*T*T*G*G*A*+C | 13176 |
| 12 | A43012H | ATACAAAGAACTTCTCG | +A*+T*+A*+C*A*A*A*G*A*A*C*T*T*C*+T*+C*+G | 19603 |
| 13 | A43014H | TCGTCCATTGTCACCTT | +T*+C*+G*T*C*C*A*T*T*G*T*C*A*C*+C*+T*+T | 22662 |
| 14 | A43015H | AGGAAGTCGTCCATTGT | +A*+G*+G*A*A*G*T*C*G*T*C*C*A*T*+T*+G*+T | 22668 |
| 15 | A43016H | GGTTGTGCAGCATATGC | +G*+G*+T*T*G*T*G*C*A*G*C*A*T*A*+T*+G*+C | 22725 |
| 16 | A43017H | AATCGTGAGTGCTTGTT | +A*+A*+T*C*G*T*G*A*G*T*G*C*T*T*+G*+T*+T | 25175 |
| 17 | A43020H | CCTTCATGTTTAGCTGC | +C*+C*+T*T*C*A*T*G*T*T*T*A*G*C*+T*+G*+C | 28690 |
| 18 | A43021H | GAGCAGTGGCCTTACAT | +G*+A*+G*C*A*G*T*G*G*C*C*T*T*A*+C*+A*+T | 29239 |
| 19 | A43022H | CTCCAGTCGGTACTCCA | +C*+T*+C*C*A*G*T*C*G*G*T*A*C*T*C*+C*+A | 29316 |
| 20 | A43023H | TCGGAACAGGTACATTT | +T*+C*+G*G*A*A*C*A*G*G*T*A*C*A*+T*+T*+T | 33154 |
| 21 | A43024H | GATCGGAACAGGTACAT | +G*+A*+T*C*G*G*A*A*C*A*G*G*T*A*+C*+A*+T | 33156 |
| 22 | A43025H | TCAAATTAGCCACTCGC | +T*+C*+A*+A*A*T*T*A*G*C*C*A*C*T*C*+G*+C | 34604 |
| 23 | A43026H | CATCATTGATACGGCTC | +C*+A*+T*C*A*T*T*G*A*T*A*C*G*G*+C*+T*+C | 36752 |
| 24 | A43027H | GTTGTCATTCAGACGGA | +G*+T*+T*G*T*C*A*T*T*C*A*G*A*C*+G*+G*+A | 36772 |
| 25 | A43028H | TAGGAGGTCCAAGTGTT | +T*+A*+G*G*A*G*G*T*C*C*A*A*G*T*+G*+T*+T | 36815 |
| 26 | A43029H | TCGATGGAGGCATAAGG | +T*+C*+G*A*T*G*G*A*G*G*C*A*T*A*+A*+G*+G | 38976 |
| 27 | A43030H | ATATCGATGGAGGCATA | +A*+T*+A*T*C*G*A*T*G*G*A*G*G*C*+A*+T*+A | 38979 |
| 28 | A43031H | CCTTTGCTAATATCGAT | +C*+C*+T*T*T*G*C*T*A*A*T*A*T*C*+G*+A*+T | 38988 |
| 29 | A43032H | TCGATTCCAAACATCAC | +T*+C*+G*A*T*T*C*C*A*A*A*C*A*T*+C*+A*+C | 39587 |
| 30 | A43033H | TAGACCTGTCACCTTGA | +T*+A*+G*A*C*C*T*G*T*C*A*C*C*T*+T*+G*+A | 39615 |
| 31 | A43034H | GTTGATCAAGCACCTTG | +G*+T*+T*G*A*T*C*A*A*G*C*A*C*C*+T*+T*+G | 39681 |
| 32 | A43035H | ATGAGTTTCTATCAGGC | +A*+T*+G*+A*G*T*T*T*C*T*A*T*C*+A*+G*+G*+C | 39753 |
| 33 | A43036H | GAGTGTGTAAATCTAGC | +G*+A*+G*T*G*T*G*T*A*A*A*T*C*T*+A*+G*+C | 39912 |
| 34 | A43037Hi | TCTTACCGCTTAATGGA | +T*+C*+T*T*A*C*C*G*C*T*T*A*A*T*+G*+G*+A | 15729 |
| 35 | A43038Hi | TTAGGTGAAGCGTTCCT | +T*+T*+A*G*G*T*G*A*A*G*C*G*T*T*+C*+C*+T | 16056 |
| 36 | A43039Hi | AATGATGCGAGAGGATC | +A*+A*+T*G*A*T*G*C*G*A*G*A*G*G*+A*+T*+C | 1345 |
| 37 | A43040Hi | TAAGTGTGGTAGGCTAG | +T*+A*+A*G*T*G*T*G*G*T*A*G*G*C*+T*+A*+G | 1489 |
| 38 | A43041Hi | AAGCTCTAGAGACCACG | +A*+A*+G*C*T*C*T*A*G*A*G*A*C*C*+A*+C*+G | 1513 |
| 39 | A43042Hi | CGACTTGTACCTGTGTG | +C*+G*+A*C*T*T*G*T*A*C*C*T*G*T*+G*+T*+G | 1727 |
| 40 | A43043Hi | AGCTTTGGAACTAGTTT | +A*+G*+C*T*T*T*G*G*A*A*C*T*A*G*+T*+T*+T | 17000 |
| 41 | A43044Hi | GGTCCTATCAACCAGAT | +G*+G*+T*C*C*T*A*T*C*A*A*C*C*A*+G*+A*+T | 17257 |
| 42 | A43045Hi | AGGATTCGAGTACAGT | +A*+G*+G*A*T*T*C*G*A*G*T*A*C*+A*+G*+T | 29673 |
| 43 | A43046Hi | CACGTTGTTTGAGATAA | +C*+A*+C*G*T*T*G*T*T*T*G*A*G*A*+T*+A*+A | 2575 |
| 44 | A43047Hi | GTTTACTTCCGAAGCTA | +G*+T*+T*T*A*C*T*T*C*C*G*A*A*G*+C*+T*+A | 3364 |
| 45 | A43048Hi | CTAACTGTACCGCTTC | +C*+T*+A*A*C*T*G*T*A*C*C*G*C*+T*+T*+C | 31018 |
| 46 | A43049Hi | GTAATGAGCCACACAAG | +G*+T*+A*A*T*G*A*G*C*C*A*C*A*C*+A*+A*+G | 3905 |
| 47 | A43050Hi | TTGACTACGCATGTGAC | +T*+T*+G*A*C*T*A*C*G*C*A*T*G*T*+G*+A*+C | 3950 |
| 48 | A43051Hi | TGATAACACACACGGAT | +T*+G*+A*T*A*A*C*A*C*A*C*A*C*G*+G*+A*+T | 18996 |
| 49 | A43052Hi | ATTAAAGACGGCCTCTG | +A*+T*+T*A*A*A*G*A*C*G*G*C*C*T*+C*+T*+G | 4282 |
| 50 | A43053Hi | GATCTGCCGAGACTAA | +G*+A*+T*C*T*G*C*C*G*A*G*A*C*+T*+A*+A | 32021 |
| 51 | A43054Hi | TACATACAGAAAGCGGC | +T*+A*+C*A*T*A*C*A*G*A*A*A*G*C*+G*+G*+C | 4614 |
| 52 | A43055Hi | AGTTGTGTAAGTATCAG | +A*+G*+T*T*G*T*G*T*A*A*G*T*A*T*+C*+A*+G | 19750 |
| 53 | A43056Hi | TAGGAGTCAGATGAGTA | +T*+A*+G*G*A*G*T*C*A*G*A*T*G*A*+G*+T*+A | 4714 |
| 54 | A43057Hi | AACGCCAATGGATGCAT | +A*+A*+C*G*C*C*A*A*T*G*G*A*T*G*+C*+A*+T | 19970 |
| 55 | A43058Hi | CATTACCATACAACGCC | +C*+A*+T*T*A*C*C*A*T*A*C*A*A*C*+G*+C*+C | 19981 |
| 56 | A43059Hi | AAGTCGTTGTCCTTAGA | +A*+A*+G*T*C*G*T*T*G*T*C*C*T*T*+A*+G*+A | 4928 |
| 57 | A43060Hi | TCGATCCAAGCGTATTT | +T*+C*+G*A*T*C*C*A*A*G*C*G*T*A*+T*+T*+T | 20121 |
| 58 | A43061Hi | CTTACGACATGTACCAC | +C*+T*+T*A*C*G*A*C*A*T*G*T*A*C*+C*+A*+C | 20147 |
| 59 | A43062Hi | GTCTCTCTTACGACATG | +G*+T*+C*T*C*T*C*T*T*A*C*G*A*C*+A*+T*+G | 20153 |
| 60 | A43063Hi | GTAAAGCACGGTCTGAT | +G*+T*+A*A*A*G*C*A*C*G*G*T*C*T*+G*+A*+T | 20230 |
| 61 | A43064Hi | CGATCCAAGCGTATTT | +C*+G*+A*T*C*C*A*A*G*C*G*T*A*+T*+T*+T | 20121 |
| 62 | A43065Hi | CCGACTCTGTGTATCTG | +C*+C*+G*A*C*T*C*T*G*T*G*T*A*T*+C*+T*+G | 20287 |
| 63 | A43066Hi | GCCTAACTTGCCGACTT | +G*+C*+C*T*A*A*C*T*T*G*C*C*G*A*+C*+T*+T | 20371 |
| 64 | A43067Hi | TAGCTGCCGTGACTTAG | +T*+A*+G*C*T*G*C*C*G*T*G*A*C*T*+T*+A*+G | 5495 |
| 65 | A43068Hi | ACTCGTAAGACACATT | +A*+C*+T*C*G*T*A*A*G*A*C*A*C*+A*+T*+T | 33085 |
| 66 | A43069Hi | CTACTCCGCTGAAGGTC | +C*+T*+A*C*T*C*C*G*C*T*G*A*A*G*+G*+T*+C | 5822 |
| 67 | A43070Hi | TTTAGTCCTCTACTCCG | +T*+T*+T*A*G*T*C*C*T*C*T*A*C*T*+C*+C*+G | 5831 |
| 68 | A43071Hi | TTAAGCCTCACTCTAGT | +T*+T*+A*A*G*C*C*T*C*A*C*T*C*T*+A*+G*+T | 21179 |
| 69 | A43072Hi | TTACCGCCTACTGTATG | +T*+T*+A*C*C*G*C*C*T*A*C*T*G*T*+A*+T*+G | 21237 |
| 70 | A43073Hi | CACCTTACCTAGGCATA | +C*+A*+C*C*T*T*A*C*C*T*A*G*G*C*+A*+T*+A | 6248 |
| 71 | A43074Hi | ATGGACACCTTACCTAG | +A*+T*+G*G*A*C*A*C*C*T*T*A*C*C*+T*+A*+G | 6253 |
| 72 | A43075Hi | GACAATGTGGAAGTCGG | +G*+A*+C*A*A*T*G*T*G*G*A*A*G*T*+C*+G*+G | 21891 |
| 73 | A43076Hi | GCACGCTGTTTGGTATT | +G*+C*+A*C*G*C*T*G*T*T*T*G*G*T*+A*+T*+T | 22138 |
| 74 | A43077Hi | TTGACGTTCTAGTGCTC | +T*+T*+G*A*C*G*T*T*C*T*A*G*T*G*+C*+T*+C | 22206 |
| 75 | A43078Hi | GATTGTCTATATGCGAA | +G*+A*+T*T*G*T*C*T*A*T*A*T*G*C*+G*+A*+A | 7145 |
| 76 | A43079Hi | CTACATCTGGCGGAAC | +C*+T*+A*C*A*T*C*T*G*G*C*G*G*+A*+A*+C | 34890 |
| 77 | A43080Hi | TCTCCGCTACAAGAACT | +T*+C*+T*C*C*G*C*T*A*C*A*A*G*A*+A*+C*+T | 22391 |
| 78 | A43081Hi | CTCTTGTTCTGGTTCCG | +C*+T*+C*T*T*G*T*T*C*T*G*G*T*T*+C*+C*+G | 23333 |
| 79 | A43082Hi | GGACCGTCTATGCCATA | +G*+G*+A*C*C*G*T*C*T*A*T*G*C*C*+A*+T*+A | 7874 |
| 80 | A43083Hi | AAGTGATGCGGTAGTAT | +A*+A*+G*T*G*A*T*G*C*G*G*T*A*G*+T*+A*+T | 8163 |
| 81 | A43084Hi | TCATCGACCAAATGCTC | +T*+C*+A*T*C*G*A*C*C*A*A*A*T*G*+C*+T*+C | 23744 |
| 82 | A43085Hi | ACTGATCTAATATCATC | +A*+C*+T*G*A*T*C*T*A*A*T*A*T*C*+A*+T*+C | 24094 |
| 83 | A43086Hi | ACCATGTCTAATACTAT | +A*+C*+C*A*T*G*T*C*T*A*A*T*A*C*+T*+A*+T | 24225 |
| 84 | A43087Hi | TAGGCCTTGTAGTTGAG | +T*+A*+G*G*C*C*T*T*G*T*A*G*T*T*+G*+A*+G | 9462 |
| 85 | A43088Hi | TGTCTGCTACGTGATGC | +T*+G*+T*C*T*G*C*T*A*C*G*T*G*A*+T*+G*+C | 24730 |
| 86 | A43089Hi | TCAGCTCACTATTCAGG | +T*+C*+A*G*C*T*C*A*C*T*A*T*T*C*+A*+G*+G | 9811 |
| 87 | A43090Hi | GAACGTGCCTAACCAT | +G*+A*+A*C*G*T*G*C*C*T*A*A*C*+C*+A*+T | 37768 |
| 88 | A43091Hi | CGTAGTGCTGCAACCAT | +C*+G*+T*A*G*T*G*C*T*G*C*A*A*C*+C*+A*+T | 25915 |
| 89 | A43092Hi | GATCTGATTCCATGTGC | +G*+A*+T*C*T*G*A*T*T*C*C*A*T*G*+T*+G*+C | 26444 |
| 90 | A43093Hi | GAGACTTGAGCTCCTAG | +G*+A*+G*A*C*T*T*G*A*G*C*T*C*C*+T*+A*+G | 26588 |
| 91 | A43094Hi | GCAGCGCAATTCTGATC | +G*+C*+A*G*C*G*C*A*A*T*T*C*T*G*+A*+T*+C | 11796 |
| 92 | A43095Hi | GTTTGAAGGTTCACGTA | +G*+T*+T*T*G*A*A*G*G*T*T*C*A*C*+G*+T*+A | 12102 |
| 93 | A43096Hi | TGTGACAAGGTAACTCA | +T*+G*+T*G*A*C*A*A*G*G*T*A*A*C*+T*+C*+A | 12127 |
| 94 | A43097Hi | CCTATCTCCGGCTACTT | +C*+C*+T*A*T*C*T*C*C*G*G*C*T*A*+C*+T*+T | 12642 |
| 95 | A43098Hi | AAGAGCCAAGTACACGA | +A*+A*+G*A*G*C*C*A*A*G*T*A*C*A*+C*+G*+A | 13230 |
| 96 | A43099Hi | GAGCTTAGCCAATCAAC | +G*+A*+G*C*T*T*A*G*C*C*A*A*T*C*+A*+A*+C | 13531 |
| 97 | A43100Hi | TCACTTATACGATTCCA | +T*+C*+A*C*T*T*A*T*A*C*G*A*T*T*+C*+C*+A | 13697 |
| 98 | A43101Hi | CGAGGTATGGCTGTGGA | +C*+G*+A*G*G*T*A*T*G*G*C*T*G*T*+G*+G*+A | 13854 |
| 99 | A43102Hi | TTCGTGTACTTAACTTG | +T*+T*+C*G*T*G*T*A*C*T*T*A*A*C*+T*+T*+G | 15124 |
| | | | | Position on mRNA |
| | | Antisense Sequence | Antisense Sequence 5′-3′ with PTO (*) | (NM_001371415; |
| | Name | 5′-3′ | and LNA (+) | SEQ ID NO. 1) |
| 100 | A43004Hi** | GAATTGTGTTCAACCGT | +G*+A*+A*T*T*G*T*G*T*T*C*A*A*C*+C*+G*+T | 391 |
| 101 | A43011Hi** | GGCCTGGTCCACCATTG | +G*+G*C*C*T*G*G*T*C*C*A*C*C*A*T*T*+G | 936 |
| 102 | A43013Hi** | TAAGGATCCTGAAGTCG | +T*+A*+A*G*G*A*T*C*C*T*G*A*A*+G*+T*+C*+G | 1111 |
| 103 | A43018Hi** | ACTATCTCTCGCTTCAT | +A*+C*+T*A*T*C*T*C*T*C*G*C*T*T*+C*+A*+T | 1487 |
| 104 | A43019Hi** | GTCCTTGTGTAATATCG | +G*+T*+C*C*T*T*G*T*G*T*A*A*T*A*+T*+C*+G | 1589 |
| 105 | R01011 | GATCATTCGCGGACAAC | +G*+A*+T*C*A*T*T*C*G*C*G*G*A*C*+A*+A*+C | Control |
| 106 | Neg1 | CGTTTAGGCTATGTACTT | +C*+G*+T*T*T*A*G*G*C*T*A*T*G*T*A*+C*+T*+T | Control |

An “H” after the ASO ID indicates a human ACE2-specific sequence that binds to an exonic region of the pre-mRNA and a “Hi” after the ASO ID indicates a human ACE2-specific sequence that binds to an intronic region of the pre-mRNA.
**= exon spanning oligo, position depicted in Table 1 indicates positions on mRNA of SEQ ID NO. 1 (RefSeq ID NM_001371415) for exon spanning oligonucleotides.

The oligonucleotides of the present invention hybridize for example with mRNA and/or pre-mRNA of human ACE2 of SEQ ID NO. 1 and SEQ ID NO.2, respectively. Such oligonucleotides are called ACE2 antisense oligonucleotides. Oligonucleotides of the present invention, which are for example antisense oligonucleotides, are shown in Table 1. The present invention further refers to oligonucleotides such as antisense oligonucleotides having 80 to 99%, 85 to 98%, 90 to 95 or 93% sequence homology to an oligonucleotide of Table 1.

Each nucleotide of the sequence can be modified, wherein ASOs of the present invention preferably comprise a core of 6 to 8 unmodified nucleotides. ASOs of the present invention comprise for example one or more modified nucleotides, e.g., 1, 2, 3, 4 or 5 nucleotides at the 5′- and/or 3′-end of the oligonucleotide, i.e., on the 5′- and/or 3′-side of the core. The 5′- and 3′-end are modified identically or differently. If the 5′- and 3′-ends are modified identically the nucleotides are modified at the same positions counted from the 5′- and 3′-end (in each case starting the counting with 1 from the end), respectively, having the same modification for example LNA-modification. If the 5′- and 3′-ends are modified differently the position of the modified nucleotide and/or the type of modification at the 5′- and 3′-ends differ; the type of nucleotide modification is the same (e.g., LNA) or different. Modified nucleotides such as LNA-modified nucleotides need not to follow in a row, but may be separated by one or more unmodified nucleotides. In the following some modification patterns at the 5′- and 3′-end of the ASOs of the present invention are described, wherein an unmodified nucleotide is indicated by “_” and the figure refers to the number of modified nucleotides such as LNA-modified nucleotides in a row. The modified nucleotide(s) is/are at any position of the 5′- and/or 3′-end of the ASO as shown in the following Table 2:

| LNA modification at the 5′- | LNA modification at the 3′- | |
|---|---|---|
| side of the core | side of the core | Abbreviation |
| 3 | 3 | 3 + 3 |
| 3 | 2 | 3 + 2 |
| 2 | 3 | 2 + 3 |
| 1_1 | 3 | 1_1 + 3 |
| 1_1 | 2 | 1_1 + 2 |
| 1_1 | 1_1 | 1_1 + 1_1 |
| 3 | 1_1 | 3 + 1_1 |
| 2 | 1_1 | 2 + 1_1 |
| 2 | 2 | 2 + 2 |
| 4 | 3 | 4 + 3 |
| 4 | 2 | 4 + 2 |
| 4 | 1_1 | 4 + 1_1 |
| 2_1 | 3 | 2_1 + 3 |
| 2_1 | 1_1 | 2_1 + 1_1 |
| 2_1 | 2 | 2_1 + 2 |
| 3 | 4 | 3 + 4 |
| 2 | 4 | 2 + 4 |
| 1_1 | 4 | 1_1 + 4 |
| 3 | 1_2 | 3 + 1_2 |
| 1_1 | 1_2 | 1_1 + 1_2 |
| 2 | 1_2 | 2 + 1_2 |

Typical modification patterns of each ASO of the present invention, comprising for example LNA-modified nucleotides, are shown for example in the following Table 3 which indicates specific positions of the LNA modifications at the 5′- and 3′-end of each ASO:

| Position of the modification | Position of the modification | |
|---|---|---|
| at the 5′-end (counted from | at the 3′-end (counted from | |
| the 5′-end starting with 1) | the 3′-end starting with 1) | Abbreviation |
| nucleotides 1 to 5 | nucleotides 1 to 5 | 5 + 5 |
| nucleotides 1 to 4 | nucleotides 1 to 4 | 4 + 4 |
| nucleotides 1 to 3 | nucleotides 1 to 3 | 3 + 3 |
| nucleotides 1 and 2 | nucleotides 1 and 2 | 2 + 2 |
| nucleotide 1 | nucleotide 1 | 1 + 1 |
| nucleotides 1 to 5 | nucleotides 1 to 4 | 5 + 4 |
| nucleotides 1 to 4 | nucleotides 1 to 3 | 4 + 3 |
| nucleotides 1 to 3 | nucleotides 1 and 2 | 3 + 2 |
| nucleotides 1 and 2 | nucleotide 1 | 2 + 1 |
| nucleotide 1 | nucleotides 1 to 5 | 1 + 5 |
| nucleotides 1 to 5 | nucleotides 1 to 3 | 5 + 3 |
| nucleotides 1 to 4 | nucleotides 1 and 2 | 4 + 2 |
| nucleotides 1 to 3 | nucleotide 1 | 3 + 1 |
| nucleotides 1 and 2 | nucleotides 1 to 5 | 2 + 5 |
| nucleotide 1 | nucleotides 1 to 4 | 1 + 4 |
| nucleotides 1 to 5 | nucleotides 1 and 2 | 5 + 2 |
| nucleotides 1 to 4 | nucleotide 1 | 4 + 1 |
| nucleotides 1 to 3 | nucleotides 1 to 5 | 3 + 5 |
| nucleotides 1 and 2 | nucleotides 1 to 4 | 2 + 4 |
| nucleotide 1 | nucleotides 1 to 3 | 1 + 3 |
| nucleotides 1 to 5 | nucleotide 1 | 5 + 1 |
| nucleotides 1 to 4 | nucleotides 1 to 5 | 4 + 5 |
| nucleotides 1 to 3 | nucleotides 1 to 4 | 3 + 4 |
| nucleotides 1 and 2 | nucleotides 1 to 3 | 2 + 3 |
| nucleotide 1 | nucleotides 1 and 2 | 1 + 2 |
| nucleotides 1 and 2 | nucleotides 1 and 3 | 2 + 1_1 |
| nucleotides 1 and 3 | nucleotides 1 to 3 | 1_1 + 3 |
| nucleotides 1 and 3 | nucleotides 1 and 3 | 1_1 + 1_1 |
| nucleotides 1 to 3 | nucleotides 1 and 3 | 3 + 1_1 |
| nucleotides 1 to 4 | nucleotides 1 and 3 | 4 + 1_1 |
| nucleotides 1, 2 and 4 | nucleotides 1 to 3 | 2_1 + 3 |
| nucleotides 1, 2 and 4 | nucleotides 1 and 3 | 2_1 + 1_1 |
| nucleotides 1, 2 and 4 | nucleotides 1 and 2 | 2_1 + 2 |
| nucleotides 1 and 3 | nucleotides 1 to 4 | 1_1 + 4 |
| nucleotides 1 to 3 | nucleotides 1, 2 and 4 | 3 + 1_2 |
| nucleotides 1 and 3 | nucleotides 1, 2 and 4 | 1_1 + 1_2 |
| nucleotides 1 and 2 | nucleotides 1, 2 and 4 | 2 + 1_2 |

The oligonucleotides of the present invention hybridize with hybridizing active regions of SEQ ID NO.2. In the present invention surprisingly several hybridizing active regions were identified for example selected from position 145 to 944, position 945 to 1744, position 2545 to 3344, position 3345 to 4144, position 4145 to 4944, position 4945 to 5744, position 5745 to 6544, position 6545 to 7344, position 7345to 8144, position 8145 to 8944, position 8945 to 9744, position 9745 to 10544, position 11345 to 12144, position 12145 to 12944, position 129451 to 13744, position 13745 to 14544, position 14545 to 15344, position 15345 to 16144, position 16945 to 17744 position 18545 to 19344, position 19345 to 20144, position 20145 to 20944, position 20945 to 21744, position 21745 to 22544, position 22545 to 23344, position 23345 to 24144, position 24145 to 24944, position 24945 to 25744, position 25745 to 26544, position 26545 to 27344, position 28145 to 28944, position 28945 to 29744, position 30545 to 31344, position 31345 to 32144, position 32945 to 33744, position 34545 to 35344, position 36145 to 36944, position 37745 to 38544, position 38545 to 39344, position 39345 to 39928 or a combination thereof (including the terminal figures of the ranges) of ACE2 pre-mRNA for example of SEQ ID NO.2. These regions and the oligonucleotides hybridizing in the different regions are shown in the following Table 4:

| Region of | First position on | |
|---|---|---|
| SEQ ID NO. 2 | SEQ ID NO. 2 | SEQ ID NO. |
| 145-944 | | |
| A43002H | 145 | 4 |
| A43003H | 151 | 5 |
| 945-1744 | | |
| A43039Hi | 1345 | 36 |
| A43040Hi | 1489 | 37 |
| A43041Hi | 1513 | 38 |
| A43042Hi | 1727 | 39 |
| 2545-3344 | | |
| A43046Hi | 2575 | 43 |
| 3345-4144 | | |
| A43047Hi | 3364 | 44 |
| A43049Hi | 3905 | 46 |
| A43050Hi | 3950 | 47 |
| 4145-4944 | | |
| A43052Hi | 4282 | 49 |
| A43054Hi | 4614 | 51 |
| A43056Hi | 4714 | 53 |
| A43059Hi | 4928 | 56 |
| 4945-5744 | | |
| A43067Hi | 5495 | 64 |
| 5745-6544 | | |
| A43069Hi | 5822 | 66 |
| A43070Hi | 5831 | 67 |
| A43073Hi | 6248 | 70 |
| A43074Hi | 6253 | 71 |
| 6545-7344 | | |
| A43078Hi | 7145 | 75 |
| 7345-8144 | | |
| A43082Hi | 7874 | 79 |
| 8145-8944 | | |
| A43005H | 8700 | 6 |
| A43083Hi | 8163 | 80 |
| 8945-9744 | | |
| A43006H | 9133 | 7 |
| A43007H | 9169 | 8 |
| A43087Hi | 9462 | 84 |
| 9745-10544 | | |
| A43089Hi | 9811 | 86 |
| 11345-12144 | | |
| A43008H | 11571 | 9 |
| A43009H | 11576 | 10 |
| A43094Hi | 11796 | 91 |
| A43095Hi | 12102 | 92 |
| A43096Hi | 12127 | 93 |
| 12145-12944 | | |
| A43097Hi | 12642 | 94 |
| 12945-13744 | | |
| A43010H | 13176 | 11 |
| A43098Hi | 13230 | 95 |
| A43099Hi | 13531 | 96 |
| A43100Hi | 13697 | 97 |
| 13745-14544 | | |
| A43101Hi | 13854 | 98 |
| 14545-15344 | | |
| A43102Hi | 15124 | 99 |
| 15345-16144 | | |
| A43037Hi | 15729 | 34 |
| A43038Hi | 16056 | 35 |
| 16945-17744 | | |
| A43043Hi | 17000 | 40 |
| A43044Hi | 17257 | 41 |
| 18545-19344 | | |
| A43051Hi | 18996 | 48 |
| 19345-20144 | | |
| A43012H | 19603 | 12 |
| A43055Hi | 19750 | 52 |
| A43057Hi | 19970 | 54 |
| A43058Hi | 19981 | 55 |
| A43060Hi | 20121 | 57 |
| A43064Hi | 20121 | 61 |
| 20145-20944 | | |
| A43061Hi | 20147 | 58 |
| A43062Hi | 20153 | 59 |
| A43063Hi | 20230 | 60 |
| A43065Hi | 20287 | 62 |
| A43066Hi | 20371 | 63 |
| 20945-21744 | | |
| A43071Hi | 21179 | 68 |
| A43072Hi | 21237 | 69 |
| 21745-22544 | | |
| A43075Hi | 21891 | 72 |
| A43076Hi | 22138 | 73 |
| A43077Hi | 22206 | 74 |
| A43080Hi | 22391 | 77 |
| 22545-23344 | | |
| A43014H | 22662 | 13 |
| A43015H | 22668 | 14 |
| A43016H | 22725 | 15 |
| A43081Hi | 23333 | 78 |
| 23345-24144 | | |
| A43084Hi | 23744 | 81 |
| A43085Hi | 24094 | 82 |
| 24145-24944 | | |
| A43086Hi | 24225 | 83 |
| A43088Hi | 24730 | 85 |
| 24945-25744 | | |
| A43017H | 25175 | 16 |
| 25745-26544 | | |
| A43091Hi | 25915 | 88 |
| A43092Hi | 26444 | 89 |
| 26545-27344 | | |
| A43093Hi | 26588 | 90 |
| 28145-28944 | | |
| A43020H | 28690 | 17 |
| 28945-29744 | | |
| A43021H | 29239 | 18 |
| A43022H | 29316 | 19 |
| A43045Hi | 29673 | 42 |
| 30545-31344 | | |
| A43048Hi | 31018 | 45 |
| 31345-32144 | | |
| A43053Hi | 32021 | 50 |
| 32945-33744 | | |
| A43023H | 33154 | 20 |
| A43024H | 33156 | 21 |
| A43068Hi | 33085 | 65 |
| 34545-35344 | | |
| A43025H | 34604 | 22 |
| A43079Hi | 34890 | 76 |
| 36145-36944 | | |
| A43026H | 36752 | 23 |
| A43027H | 36772 | 24 |
| A43028H | 36815 | 25 |
| 37745-38544 | | |
| A43090Hi | 37768 | 87 |
| 38545-39344 | | |
| A43029H | 38976 | 26 |
| A43030H | 38979 | 27 |
| A43031H | 38988 | 28 |
| 39345-39928 | | |
| A43032H | 39587 | 29 |
| A43033H | 39615 | 30 |
| A43034H | 39681 | 31 |
| A43035H | 39753 | 32 |
| A43036H | 39912 | 33 |

Table 4 shows some hybridizing active regions and antisense oligonucleotides hybridizing in these regions.

In some embodiments, the oligonucleotide of the present invention reduces the amount of ACE2 mRNA and/or the ACE2 protein expression for example about 30% -100%, 35% -99%, 40%-98%, 45%-97%, 50%-96%, 55%-95%, 60%-90%, 65%-85%, 70% -80% or at least about 30%, 35%, 40%, 45%, 50%, 55%, 60%, 65%, 70%, 75%, 80%, 85%, 90%, 92%, 94%, 95%, 96%, 97%, 98%, 99% or 100% compared to an untreated control. The reduction of the amount (level) of the ACE2 mRNA and/or ACE2 protein expression is determined by the comparison of the amount of the ACE2 mRNA and/or ACE2 protein expression in a sample treated with an oligonucleotide of the present invention and a corresponding untreated control. The untreated control is for example ACE2, ACE2 mRNA, ACE2 pre-mRNA expression or a combination thereof in a subject before an oligonucleotide of the present invention is administered or an untreated sample such as a cell. The untreated sample is for example taken from a subject before an oligonucleotide of the present invention is administered.

The oligonucleotide of the present invention reduces the amount (level) of ACE2 mRNA and/or the expression of ACE2 protein expression at a nanomolar or micromolar concentration for example at a concentration of 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900 or 950 nM, or 1, 10 or 100 μM.

The oligonucleotide of the present invention is for example used in a concentration of 1, 20 3, 5, 9, 10, 15, 27, 30, 40, 50, 75, 82, 100, 250, 300, 500, or 740 nM, or 1, 2.2, 3, 5, 6.6 or 1 μM.

The present invention also refers to a pharmaceutical composition comprising an oligonucleotide of the present invention and a pharmaceutically acceptable carrier, excipient, stimulant such as adjuvant and/or dilutant. Optionally, the pharmaceutical composition further comprises another oligonucleotide which is different from the present invention, an antibody and/or a small molecule.

Adjuvants are for example aluminium such as amorphous aluminium hydroxyphosphate sulfate (AAHS), aluminum hydroxide, aluminum phosphate, potassium aluminum sulfate (Alum), monophosphoryl lipid A (MPL) and optionally aluminum salt, squalene for example in an oil in water emulsion, monophosphoryl lipid A (MPL) and optionally a natural compound extracted from the Chilean soapbark tree (e.g., QS-21) for example in a liposomal formulation or a synthetic form of DNA that mimics bacterial and viral genetic material such as cytosine phosphoguanine (CpG).

The oligonucleotide or the pharmaceutical composition of the present invention is for example for use in a method of preventing and/or treating a viral disease. The use of the oligonucleotide or the pharmaceutical composition of the present invention in a method of preventing and/or treating a viral disease is for example combined with another therapy of a viral disease. ACE2 is for example an entrance for viruses such as coronaviruses to a cell. Reduction of ACE2 reduces or even avoids further entrance of a virus into a cell.

Thus, an oligonucleotide of the present invention protects a cell for example a nasal cell such as a nasal epithelial cell, a lung cell for example comprising cells of the bronchia such as epithelial cells, cells of the mucosa, or goblet cells, or of the alveolus such as fibrocytes, macrophages, alveolar epithelial cells (e.g., Type I, Type II) from an infection with a virus such as Sars-CoV-2. The oligonucleotides of the present invention interact for example with cells of the lung and/or throat and reduce ACE2 expression in these cells.

The ACE2 mRNA and protein level, respectively, can be measured by any standard method such as quantitative real time PCR or QuantiGene assay, immunohistochemistry or western blot known to a person skilled in the art. An oligonucleotide or a pharmaceutical composition of the present invention is administered locally or systemically for example orally, via inhalation for example in aerosol or powder form, sublingually, nasally, subcutaneously, intravenously, intraperitoneally, intramuscularly, intratumoral, intrathecal, transdermal, and/or rectal. Alternatively or in combination ex vivo treated immune cells are administered.

One or more, two or more, three or more, four or more, or five or more oligonucleotides of the present invention are for example administered together, at the same time point, e.g., in a pharmaceutical composition or separately, or on staggered intervals.

Alternatively, the one or more, two or more, three or more, four or more or five or more oligonucleotides of the present invention are administered together with an active agent such as an antiviral active agent, an immune stimulating agent, disease specific agent or an agent that reverses infection-mediated immunosuppression or a combination thereof. Alternatively or additionally, an agent that ameliorates infection-mediated organ damage or symptoms of viral disease, an antiviral active agent, an immune stimulating agent, disease specific agent or an agent that reverses infection-mediated immunosuppression or a combination thereof may be administered.

The active agent is for example another oligonucleotide (i.e., different from the present invention), an antibody, a small molecule and/or a therapeutic such as a nucleoside analogue, a nucleotide analogue, a protease inhibitor, an ACE2 blocking peptide, an ACE2 fusion protein, a recombinant ACE2 such as Remdesivir, Umifenovir, Favipiravir, Chloroquine, Hydroxychloroquine, Dexamethasone, Lopinavir, Ritonavir, Darunavir, APN01, Favilavir, Molnupiravir, SNG001, Tocilizumab, Anakinra or a combination thereof. The oligonucleotide and the active agent are for example administered at the same time point for example in a pharmaceutical composition or separately, or on staggered intervals.

The oligonucleotide of the present invention and the active agent interact for example with the same target such as ACE2 on the same or different level, e.g., ACE2 mRNA, ACE2 pre-mRNA and/or ACE2 protein. Alternatively, the oligonucleotide of the present invention and the active agent interact with different targets. For example the oligonucleotide of the present invention reduces for example the amount of ACE2 mRNA, ACE2 pre-mRNA and/or ACE2 protein expression and the active agent such as another oligonucleotide (i.e., different from the present invention), the antibody, the lipid and/or small molecule inhibits (antagonist) or stimulates (agonist) another target such as a factor involved in virus replication.

The oligonucleotide alone or in combination with the active agent is efficient in preventing and/or treating a viral disease such as Coronavirus Disease 2019 (COVID-19), Severe Acute Respiratory Syndrome (SARS) or Middle East Respiratory Syndrome (MERS). Thus, the oligonucleotide of the present invention is preventing and/or treating a viral infection such as SARS-CoV infection, SARS-CoV2 infection or HCoV-NL63 infection.

The oligonucleotide or pharmaceutical composition of the present invention is for example for use in combination with vaccination to prevent a viral disease. Prevention of virally caused diseases by the oligonucleotides of the present invention is an important aspect of the present invention. ACE2 is expressed by ciliated cells on the surface of the nasal mucosa, and these cells are replaced by new cells continuously. In a situation where at a hospital or elderly home, or a specific area such as a school, educational institution or home for disabled people, or similar facilities, a few viral infections such as few COVID infections have been detected, an intervention with ACE2-specific ASOs in the non-infected population may reduce apical expression of ACE2 in newly formed epithelial cells and thus, reduce or even avoid the possible viral spread in due time (which for example depends on the speed of replacement of normal epithelial cells (turn-around), then not expressing surface ACE2 anymore).

Thus, the present invention is further directed to a vaccine comprising an oligonucleotide or a pharmaceutical composition of the present invention. A vaccine against a viral disease such as SARS-CoV, SARS-CoV-2 or HCoV-NL63 include whole virus vaccine, protein(epitope)-based vaccines, viral vector vaccines, nucleic acid-based vaccines (including RNA, double-stranded DNA).

Further, the present invention relates to a kit comprising an oligonucleotide or pharmaceutical composition of the present invention and optionally technical instructions providing information on administration and/or dosage of the oligonucleotide or pharmaceutical composition. The kit may further comprise a stimulant such as an adjuvant.

The vaccine and the kit, respectively, is stored for example at −70° C. to 40° C., −18° C. to 35° C., −4° C. to 30° C., 0° C. to 25° C. or 20° C.

A subject of the present invention is for example a mammalian such as a human, dog, cat horse, cow, pig, a bird or a fish.

### EXAMPLES

The following examples illustrate different embodiments of the present invention, but the invention is not limited to these examples. The following experiments are performed on cells endogenously expressing ACE2, i.e., the cells do not represent an artificial system comprising transfected reporter constructs. Such artificial systems generally show a higher degree of inhibition and lower IC₅₀ values than endogenous systems which are closer to therapeutically relevant in vivo systems. Further, in the following experiments no transfecting agent is used, i.e., gymnotic delivery is performed. Transfecting agents are known to increase the activity of an oligonucleotide which influences the IC₅₀ value (see for example Zhang et al., Gene Therapy, 2011, 18, 326-333; Stanton et al., Nucleic Acid Therapeutics, Vol. 22, No. 5, 2012). Since artificial systems using a transfecting agent are hard or impossible to translate into therapeutic approaches and no transfection formulation has been approved so far for oligonucleotides, the following experiments are performed without any transfecting agent.

### Example 1: Design of Human ACE2-Specific Antisense Oligonucleotides (ASOs)

For the design of ASOs with specificity for exonic regions within the human ACE2 gene the ACE2 mRNA of SEQ ID NO.1 (RefSeq ID NM_001371415) was used. For ASOs with specificity for intronic regions within the human ACE2 gene the ACE2 pre-mRNA of SEQ ID NO.2 (GRCh38: Chr X:15561033:15600960:-1) as annotated in FASTA format (visible range) downloaded from https://www.ncbi.nlm.nih.gov/nuccore/NG_012575.2?from=6199&to=46126&report=fasta was used. An “H” after the ASO ID indicates a human ACE2-specific sequence that binds to an exonic region of the pre-mRNA and a “Hi” after the ASO ID indicates a human ACE2-specific sequence that binds to an intronic region of the pre-mRNA. 16 and 17 mers were designed according to in house criteria, Neg1 (described in WO2014154843 A1) and R01011 were used as non-targeting control oligonucleotides in some experiments (Table 1).

### Example 2: Target Knockdown Efficacy Screens of Human ACE2-Specific ASOs in HEK293T and 1618-K Cells

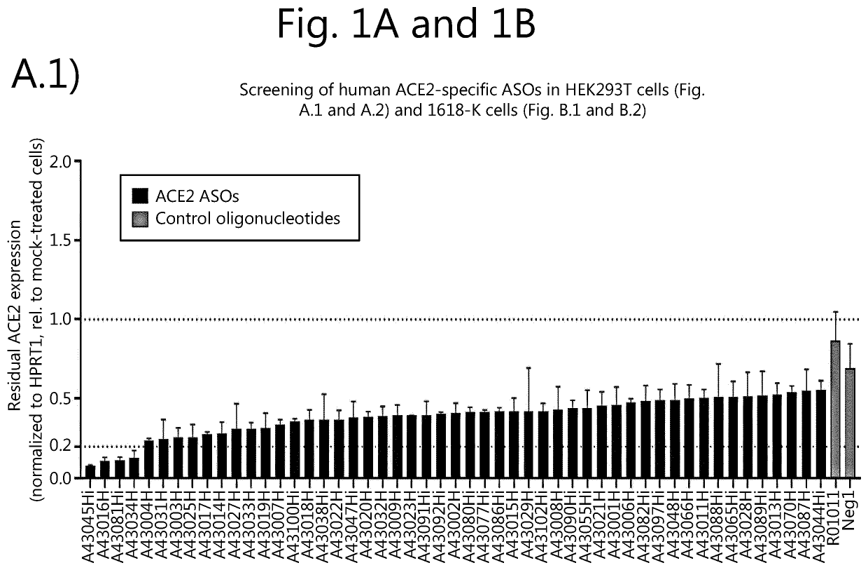

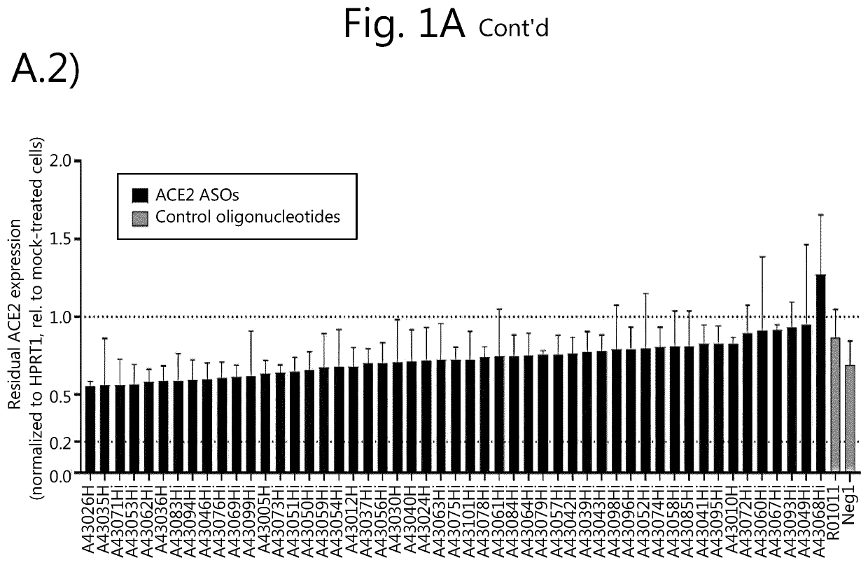

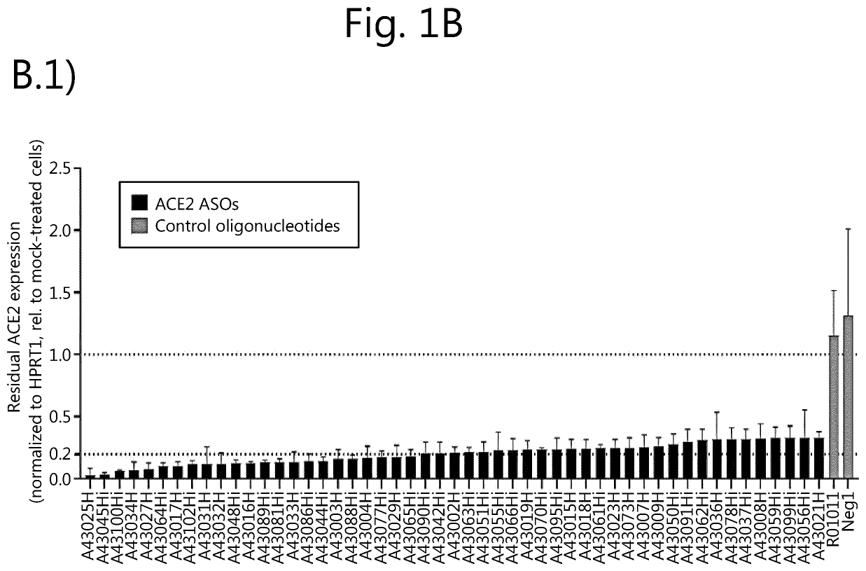

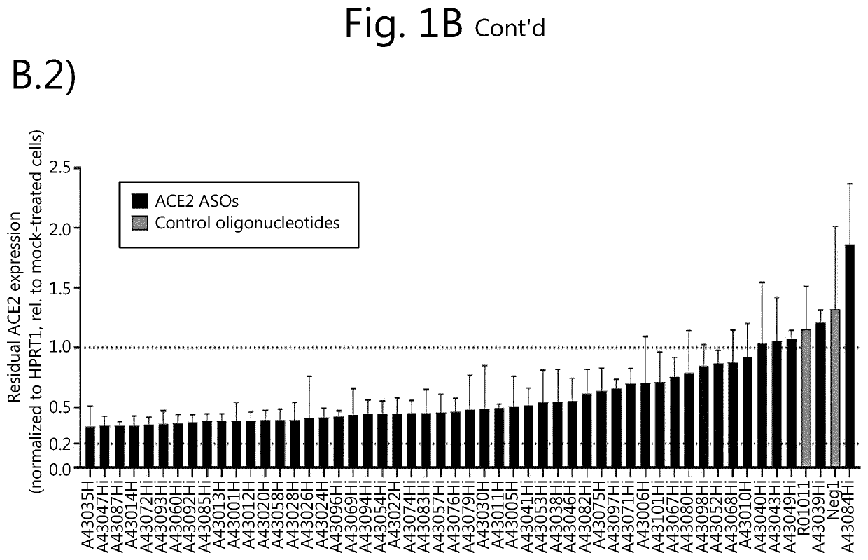

Knockdown efficacy of the ACE2-specific ASOs were tested in human HEK293T cells and 1618-K cells. The cells were treated with the respective ACE2-specific ASO or control oligonucleotide at a concentration of 10 μM. After three days treatment, cells were lyzed. ACE2 and HPRT1 mRNA expression was analyzed using the QuantiGene Singleplex assay (ThermoFisher) and the ACE2 expression values were normalized to HPRT1 values. The results of ASOs in HEK293T cells and in 1618-K cells are shown as residual ACE2 mRNA expression relative to mock-treated cells (set as 1) in FIG. 1 and Table 5 and 6. Treatment of HEK293T cells with the ASOs A43045Hi (SEQ ID NO.42), A43016H (SEQ ID NO.15), A43081Hi (SEQ ID NO.78) and A43034H (SEQ ID NO.31) resulted in a target inhibition of >80% (represented by a residual ACE2 mRNA expression of <0.2 as compared to mock-treated cells) (FIG. 1A and Table 5). As shown in FIG. 1B and Table 6, treatment with the ASOs A43025H (SEQ ID NO.22), A43045Hi (SEQ ID NO.42), A43100Hi (SEQ ID NO.97), A43034H (SEQ ID NO.31) and A43027H (SEQ ID NO.24) resulted in a target inhibition of >90% (represented by a residual ACE2 mRNA expression of <0.1 as compared to mock treated cells).

TABLE 5
List of the mean ACE2 mRNA expression values in ASO-
treated HEK293T cells compared to mock-treated cells.
Expression values are normalized to HPRT1.

| | Residual ACE2 | | Residual ACE2 |
|---|---|---|---|
| | mRNA expression | | mRNA expression |
| | compared to mock- | | compared to mock- |
| | treated cells | | treated cells |
| ASO ID | (set as 1) | ASO ID | (set as 1) |
| A43045Hi | 0.08 | A43071Hi | 0.56 |
| A43016H | 0.11 | A43053Hi | 0.57 |
| A43081Hi | 0.11 | A43062Hi | 0.58 |
| A43034H | 0.13 | A43036H | 0.59 |
| A43004H | 0.24 | A43083Hi | 0.59 |
| A43031H | 0.25 | A43094Hi | 0.59 |
| A43003H | 0.25 | A43046Hi | 0.60 |
| A43025H | 0.25 | A43076Hi | 0.61 |
| A43017H | 0.28 | A43069Hi | 0.61 |
| A43014H | 0.28 | A43099Hi | 0.62 |
| A43027H | 0.31 | A43005H | 0.64 |
| A43033H | 0.31 | A43073Hi | 0.64 |
| A43019H | 0.31 | A43051Hi | 0.65 |
| A43007H | 0.33 | A43050Hi | 0.66 |
| A43100Hi | 0.36 | A43059Hi | 0.68 |
| A43018H | 0.36 | A43054Hi | 0.68 |
| A43038Hi | 0.37 | A43012H | 0.68 |
| A43022H | 0.37 | A43037Hi | 0.70 |
| A43047Hi | 0.38 | A43056Hi | 0.70 |
| A43020H | 0.39 | A43030H | 0.71 |
| A43032H | 0.39 | A43040Hi | 0.71 |
| A43009H | 0.39 | A43024H | 0.72 |
| A43023H | 0.39 | A43063Hi | 0.72 |
| A43091Hi | 0.40 | A43075Hi | 0.73 |
| A43092Hi | 0.41 | A43101Hi | 0.73 |
| A43002H | 0.41 | A43078Hi | 0.74 |
| A43080Hi | 0.41 | A43061Hi | 0.75 |
| A43077Hi | 0.41 | A43084Hi | 0.75 |
| A43086Hi | 0.42 | A43064Hi | 0.75 |
| A43015H | 0.42 | A43079Hi | 0.76 |
| A43029H | 0.42 | A43057Hi | 0.76 |
| A43102Hi | 0.42 | A43042Hi | 0.76 |
| A43008H | 0.43 | A43039Hi | 0.78 |
| A43090Hi | 0.44 | A43043Hi | 0.78 |
| A43055Hi | 0.44 | A43098Hi | 0.79 |
| A43021H | 0.46 | A43096Hi | 0.79 |
| A43001H | 0.46 | A43052Hi | 0.80 |
| A43006H | 0.47 | A43074Hi | 0.81 |
| A43082Hi | 0.48 | A43058Hi | 0.81 |
| A43097Hi | 0.49 | A43085Hi | 0.81 |
| A43048Hi | 0.49 | A43041Hi | 0.83 |
| A43066Hi | 0.50 | A43095Hi | 0.83 |
| A43011H | 0.50 | A43010H | 0.83 |
| A43088Hi | 0.51 | A43072Hi | 0.90 |
| A43065Hi | 0.51 | A43060Hi | 0.91 |
| A43028H | 0.51 | A43067Hi | 0.92 |
| A43089Hi | 0.52 | A43093Hi | 0.93 |
| A43013H | 0.52 | A43049Hi | 0.95 |
| A43070Hi | 0.54 | A43068Hi | 1.28 |
| A43087Hi | 0.55 | R01011 | 0.87 |
| A43044Hi | 0.55 | Neg1 | 0.69 |
| A43026H | 0.56 | Mock- | 1.00 |
| | | treated cells | |
| A43035H | 0.56 | | |

TABLE 6
List of the mean ACE2 mRNA expression values in ASO-
treated 1618-K cells compared to mock-treated cells.
Expression values are normalized to HPRT1.

| | Residual ACE2 | | Residual ACE2 |
|---|---|---|---|
| | mRNA expression | | mRNA expression |
| | compared to mock- | | compared to mock- |
| | treated cells | | treated cells |
| ASO ID | (set as 1) | ASO ID | (set as 1) |
| A43025H | 0.03 | A43087Hi | 0.35 |
| A43045Hi | 0.04 | A43014H | 0.35 |
| A43100Hi | 0.06 | A43072Hi | 0.36 |
| A43034H | 0.07 | A43093Hi | 0.37 |
| A43027H | 0.08 | A43060Hi | 0.37 |
| A43064Hi | 0.11 | A43092Hi | 0.38 |
| A43017H | 0.11 | A43085Hi | 0.39 |
| A43102Hi | 0.12 | A43013H | 0.39 |
| A43031H | 0.12 | A43001H | 0.39 |
| A43032H | 0.12 | A43012H | 0.39 |
| A43048Hi | 0.12 | A43020H | 0.40 |
| A43016H | 0.13 | A43058Hi | 0.40 |
| A43089Hi | 0.13 | A43028H | 0.40 |
| A43081Hi | 0.13 | A43026H | 0.41 |
| A43033H | 0.13 | A43024H | 0.42 |
| A43086Hi | 0.14 | A43096Hi | 0.43 |
| A43044Hi | 0.14 | A43069Hi | 0.44 |
| A43003H | 0.16 | A43094Hi | 0.44 |
| A43088Hi | 0.16 | A43054Hi | 0.45 |
| A43004H | 0.17 | A43022H | 0.45 |
| A43077Hi | 0.17 | A43074Hi | 0.45 |
| A43029H | 0.18 | A43083Hi | 0.46 |
| A43065Hi | 0.18 | A43057Hi | 0.46 |
| A43090Hi | 0.20 | A43076Hi | 0.47 |
| A43042Hi | 0.20 | A43079Hi | 0.48 |
| A43002H | 0.21 | A43030H | 0.49 |
| A43063Hi | 0.21 | A43011H | 0.50 |
| A43051Hi | 0.22 | A43005H | 0.51 |
| A43055Hi | 0.23 | A43041Hi | 0.52 |
| A43066Hi | 0.23 | A43053Hi | 0.54 |
| A43019H | 0.24 | A43038Hi | 0.55 |
| A43070Hi | 0.24 | A43046Hi | 0.56 |
| A43095Hi | 0.24 | A43082Hi | 0.62 |
| A43015H | 0.24 | A43075Hi | 0.64 |
| A43018H | 0.25 | A43097Hi | 0.66 |
| A43061Hi | 0.25 | A43071Hi | 0.70 |
| A43023H | 0.25 | A43006H | 0.70 |
| A43073Hi | 0.25 | A43101Hi | 0.71 |
| A43007H | 0.26 | A43067Hi | 0.75 |
| A43009H | 0.27 | A43080Hi | 0.79 |
| A43050Hi | 0.28 | A43098Hi | 0.85 |
| A43091Hi | 0.30 | A43052Hi | 0.87 |
| A43062Hi | 0.31 | A43068Hi | 0.88 |
| A43036H | 0.32 | A43010H | 0.93 |
| A43078Hi | 0.32 | A43040Hi | 1.03 |
| A43037Hi | 0.32 | A43043Hi | 1.05 |
| A43008H | 0.32 | A43049Hi | 1.07 |
| A43059Hi | 0.33 | R01011 | 1.16 |
| A43099Hi | 0.33 | A43039Hi | 1.21 |
| A43056Hi | 0.33 | Neg1 | 1.32 |
| A43021H | 0.34 | A43084Hi | 1.86 |
| A43035H | 0.34 | Mock- | 1.00 |
| | | treated cells | |
| A43047Hi | 0.35 | | |

### Example 3: Investigation of the Concentration-Dependent Target Knockdown by Selected Human ACE2-Specific ASOs in 1618-K Cells

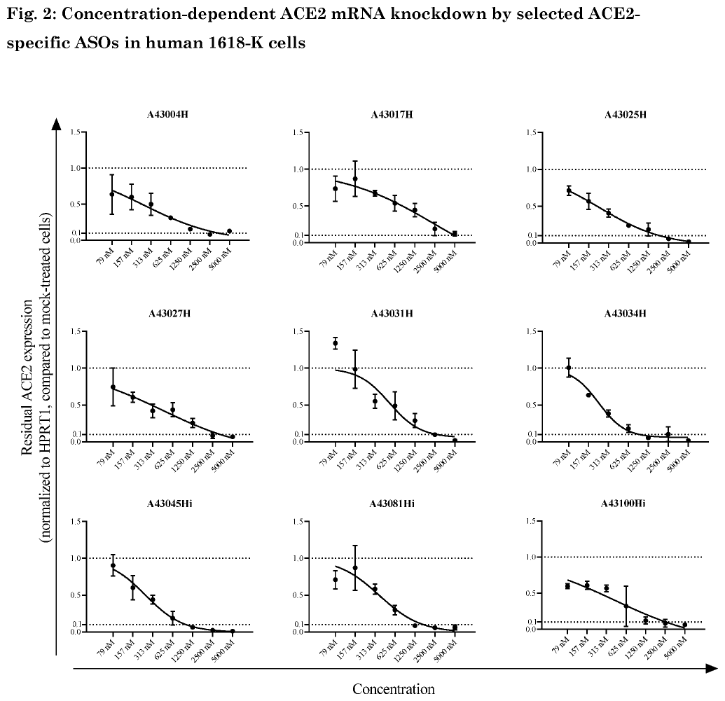

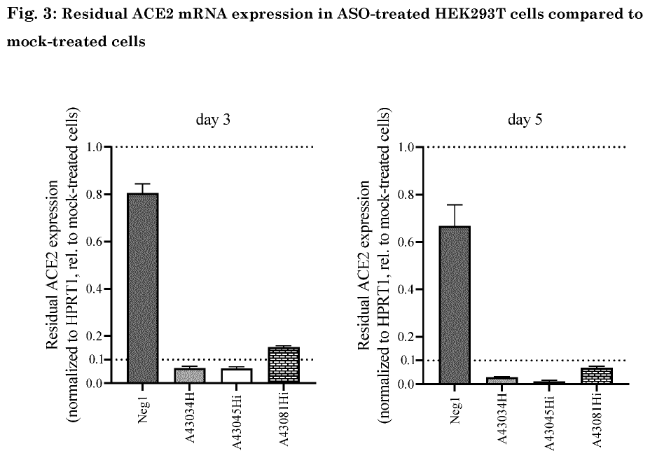

The concentration-dependent knockdown of ACE2 mRNA expression by ACE2-specific ASOs in human 1618-K cells was investigated on mRNA level and the respective IC₅₀ values were calculated. 1618-K cells were treated for three days with the respective ASO at the following concentrations: 5000 nM, 2500 nM, 1250 nM, 625 nM, 313 nM, 157 nM and 79 nM. After the three days treatment, cells were lyzed, ACE2 and HPRT1 mRNA expression was analyzed using the QuantiGene Singleplex assay (ThermoFisher) and the ACE2 expression values were normalized to HPRT1 values. The results are shown as residual ACE2 mRNA expression relative to mock-treated cells (set as 1) in FIG. 2 and Table 7. The half maximal inhibitory concentration (IC₅₀) values of the dose response curve were determined (Table 8). All ASOs dose-dependently inhibited the expression of ACE2 mRNA and had IC₅₀ values in the nanomolar range (except for A43017H (SEQ ID NO.16)).

TABLE 7
Concentration-dependent inhibition of ACE2 mRNA expression in 1618-K
cells by selected ACE2-specific ASOs after 3 days treatment.

| | | | Residual ACE2 mRNA | | | | Residual ACE2 mRNA |
|---|---|---|---|---|---|---|---|
| | | | expression compared to | | | | expression compared to |
| | | | mock-treated cells | | | | mock-treated cells |
| ASO ID | Concentration | | (set as 1) | ASO ID | Concentration | | (set as 1) |
| A43004H | 79 | nM | 0.64 | A43034H | 79 | nM | 1.01 |
| | 157 | nM | 0.60 | | 157 | nM | 0.63 |
| | 313 | nM | 0.50 | | 313 | nM | 0.38 |
| | 625 | nM | 0.31 | | 625 | nM | 0.18 |
| | 1250 | nM | 0.16 | | 1250 | nM | 0.05 |
| | 2500 | nM | 0.08 | | 2500 | nM | 0.10 |
| | 5000 | nM | 0.13 | | 5000 | nM | 0.02 |
| A43017H | 79 | nM | 0.73 | A43045Hi | 79 | nM | 0.90 |
| | 157 | nM | 0.87 | | 157 | nM | 0.60 |
| | 313 | nM | 0.67 | | 313 | nM | 0.44 |
| | 625 | nM | 0.54 | | 625 | nM | 0.19 |
| | 1250 | nM | 0.44 | | 1250 | nM | 0.06 |
| | 2500 | nM | 0.19 | | 2500 | nM | 0.02 |
| | 5000 | nM | 0.12 | | 5000 | nM | 0.01 |
| A43025H | 79 | nM | 0.71 | A43081Hi | 79 | nM | 0.71 |
| | 157 | nM | 0.57 | | 157 | nM | 0.87 |
| | 313 | nM | 0.41 | | 313 | nM | 0.58 |
| | 625 | nM | 0.24 | | 625 | nM | 0.30 |
| | 1250 | nM | 0.18 | | 1250 | nM | 0.08 |
| | 2500 | nM | 0.06 | | 2500 | nM | 0.06 |
| | 5000 | nM | 0.02 | | 5000 | nM | 0.06 |
| A43027H | 79 | nM | 0.74 | A43100Hi | 79 | nM | 0.60 |
| | 157 | nM | 0.60 | | 157 | nM | 0.61 |
| | 313 | nM | 0.42 | | 313 | nM | 0.57 |
| | 625 | nM | 0.43 | | 625 | nM | 0.32 |
| | 1250 | nM | 0.26 | | 1250 | nM | 0.12 |
| | 2500 | nM | 0.08 | | 2500 | nM | 0.08 |
| | 5000 | nM | 0.07 | | 5000 | nM | 0.06 |

| | | | |
|---|---|---|---|
| A43031H | 79 | nM | 1.34 |
| | 157 | nM | 0.99 |
| | 313 | nM | 0.55 |
| | 625 | nM | 0.49 |
| | 1250 | nM | 0.29 |
| | 2500 | nM | 0.10 |
| | 5000 | nM | 0.02 |

TABLE 8
Half maximal inhibitory concentration (IC₅₀) values and
R values of selected ACE2 ASOs after 3 days treatment.

| | IC50 | R |
|---|---|---|
| ASO ID | (nM) | squared |
| A43004H | 242.00 | 0.75 |
| A43017H | 2494.00 | 0.8 |
| A43025H | 230.20 | 0.95 |
| A43027H | 444.20 | 0.83 |
| A43031H | 515.30 | 0.81 |
| A43034H | 223.50 | 0.95 |
| A43045Hi | 244.10 | 0.93 |
| A43081Hi | 363.80 | 0.83 |
| A43100Hi | 421.60 | 0.79 |

### Example 4: Investigation of ACE2 mRNA and Protein Knockdown in HEK293T Cells Treated with ACE2-Specific ASOs Using Different Treatment Schemes

In order to further investigate the target knockdown efficacy of selected ACE2-specific ASOs at the mRNA level, HEK293T cells were treated with the ACE2-specific ASOs A43034H (SEQ ID NO.31), A43045Hi (SEQ ID NO.42) and A43081Hi (SEQ ID NO.78) as well as the control oligonucleotide Neg1 at a concentration of 101.64 for 3 or 5 days. For 5 days treatment, cells received additional ASO treatment at day 3 and were cultured for further 2 days. After 3 or 5 days treatment, the cells were lyzed. ACE2 and HPRT1 mRNA expression was analyzed using the QuantiGene Singleplex assay (ThermoFisher) and the ACE2 expression values were normalized to HPRT1 values. The results are shown as residual ACE2 mRNA expression relative to mock-treated cells (set as 1) in FIG. 3 and Table 9. Treatment of HEK293T cells with the selected ACE2-specific ASOs resulted in a target inhibition of >85% (represented by a residual ACE2 mRNA expression of <0.15 as compared to mock-treated cells) after 3 days treatment. 5 days ASO treatment reduced ACE2 mRNA expression by >93% (represented by a residual ACE2 mRNA expression of <0.07 as compared to mock-treated cells) in HEK293T cells (FIG. 3 and Table 9).

TABLE 9
List of the mean ACE2 mRNA expression values in ASO-treated
HEK293T cells compared to mock-treated cells after 3 or 5 days
ASO treatment. Expression values are normalized to HPRT1.

| 3 days treatment | | 5 days treatment | |
|---|---|---|---|
| | Residual ACE2 | | Residual ACE2 |
| | mRNA expression | | mRNA expression |
| | compared to mock- | | compared to mock- |
| | treated cells | | treated cells |
| ASO ID | (set as 1) | ASO ID | (set as 1) |
| Neg 1 | 0.80 | Neg 1 | 0.67 |
| A43034H | 0.06 | A43034H | 0.03 |
| A43045Hi | 0.06 | A43045Hi | 0.01 |
| A43081Hi | 0.15 | A43081Hi | 0.07 |

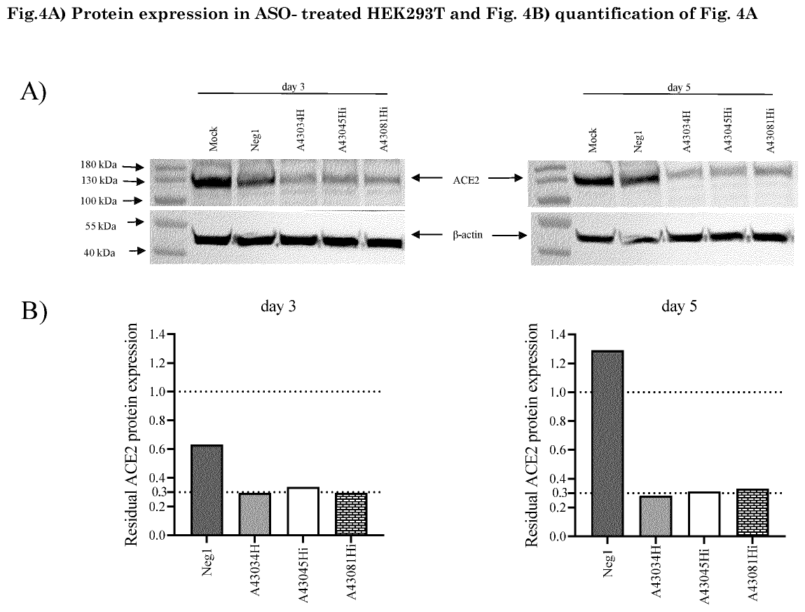

In order to investigate the knockdown efficacy of selected ACE2-specific ASOs at protein level, HEK293T cells were treated with the ACE2-specific ASOs A43034H, A43045Hi and A43081Hi as well as the control oligonucleotide Neg1 at a concentration of 5 μM for 3 or 5 days. For 5 days treatment, cells received additional ASO treatment at day 3 and were cultured for further 2 days. After 3 or 5 days treatment, the cells were lyzed. ACE2 protein expression was analyzed by Western Blot (iBlot 2 Dry Blotting System, ThermoFisher) and representative images are shown in FIG. 4A. Treatment with the different ACE2-specific ASOs for 3 or 5 days strongly reduced ACE2 protein expression as compared to mock-treated cells, whereas treatment with Neg 1 had only a modest effect after 3 days treatment but no effect after 5 days treatment in HEK293T cells (FIG. 4A). iBright Imager software was used for quantification of ACE2 and β-actin bands. Residual ACE2 expression is calculated as ACE2 band intensity compared to mock-treated cells (set as 1) normalized to the β-actin band intensity compared to mock-treated cells (set as 1) and depicted in FIG. 4B and Table 10. ASO treatment for 3 days reduced ACE2 protein expression by 70.51% (A43034H (SEQ ID NO.31), residual ACE2 expression of 0.29), 66.46% (A43045Hi (SEQ ID NO.42), residual ACE2 expression of 0.34) and 70.50% (A43081Hi (SEQ ID NO.78), residual ACE2 expression of 0.29) as compared to mock-treated cells in HEK293T cells (FIG. 4B). ASO treatment for 5 days reduced ACE2 protein expression by 71.85% (A43034H (SEQ ID NO.31), relative ACE2 expression of 0.28), 68.99% (A43045Hi (SEQ ID NO.42), relative ACE2 expression of 0.31) and 67.07% (A43081Hi (SEQ ID NO.78), relative ACE2 expression of 0.33) as compared to mock-treated cells in HEK293T cells (FIG. 4B).

TABLE 10
List of the mean Residual ACE2 protein expression values
shown as ACE2 band intensity compared to mock-treated
cells (set as 1) normalized to β-actin band intensity
compared to mock-treated cells (set as 1) in HEK293T
cells after 3 or 5 days ASO treatment.

| | |
|---|---|
| | Residual ACE2 protein |
| | expression compared to mock- |
| ASO ID | treated cells (set as 1) |

3 days treatment

| | |
|---|---|
| Neg1 | 0.63 |
| A43034H | 0.29 |
| A43045Hi | 0.34 |
| A43081Hi | 0.29 |

5 days treatment

| | |
|---|---|
| Neg1 | 1.29 |
| A43034H | 0.28 |
| A43045Hi | 0.31 |
| A43081Hi | 0.33 |

### Example 5: Investigation of ACE2 mRNA and Protein Knockdown in 1618-K Cells Treated with ACE2-Specific ASOs Using Different Treatment Schemes

In order to further investigate the target knockdown efficacy of selected ACE2-specific ASOs at the mRNA level, 1618-K cells were treated with A43034H (SEQ ID NO.31), A43045Hi (SEQ ID NO.42) and A43081Hi (SEQ ID NO.78) and the control oligonucleotide

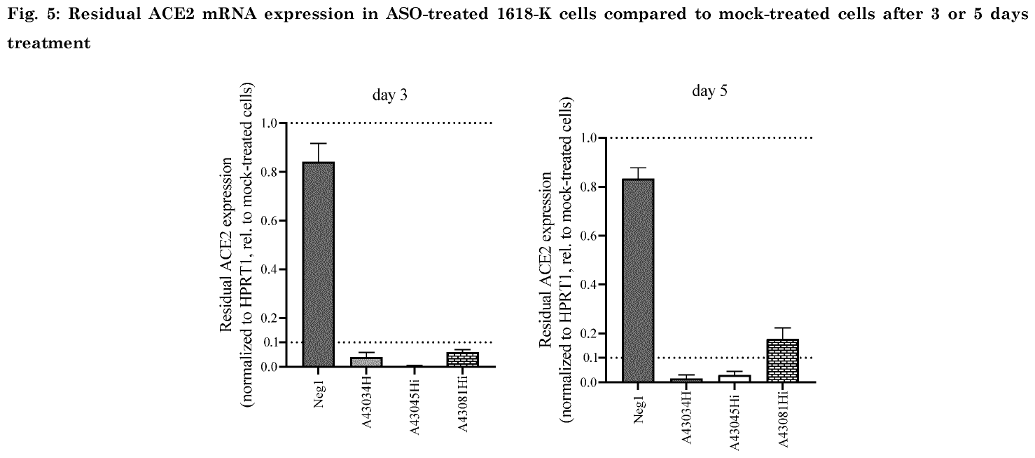

Neg1 at a concentration of 10 μM for 3 or 5 days. For 5 days treatment, cells received additional ASO treatment at day 3 and were cultured for another 2 days. After 3 or 5 days treatment, the cells were lyzed. ACE2 and HPRT1 mRNA expression was analyzed using the QuantiGene Singleplex assay (ThermoFisher) and the ACE2 expression values were normalized to HPRT1 values. The results are shown as residual ACE2 mRNA expression relative to mock-treated cells (set as 1) in FIG. 5 and Table 11. Treatment of 1618-K cells with the ACE2-specific ASOs resulted in a target inhibition of 96.74% (A43034H (SEQ ID NO.31), residual ACE2 mRNA expression of 0.03), 99.63% (A43045Hi (SEQ ID NO.42), residual ACE2 mRNA expression of 0.004) and 94.11% (A43081Hi (SEQ ID NO.78), residual ACE2 mRNA expression of 0.06) as compared to mock-treated cells (FIG. 5 and Table 11). 5 days treatment reduced ACE2 mRNA expression by 98.48% (A43034H (SEQ ID NO.31), residual ACE2 mRNA expression of 0.02), 97.03% (A43045Hi (SEQ ID NO.42), residual ACE2 mRNA expression of 0.03) and 82.24% (A43081Hi (SEQ ID NO.78), residual ACE2 mRNA expression of 0.18) as compared to mock-treated cells (FIG. 5 and Table 11).

TABLE 11
List of the mean ACE2 mRNA expression values in ASO-treated
1618-K cells compared to mock-treated cells after 3 or 5 days
ASO treatment. Expression values are normalized to HPRT1.

| 3 days treatment | | 5 days treatment | |
|---|---|---|---|
| | Residual ACE2 | | Residual ACE2 |
| | mRNA expression | | mRNA expression |
| | compared to mock- | | compared to mock- |
| | treated cells | | treated cells |
| ASO ID | (set as 1) | ASO ID | (set as 1) |
| Neg 1 | 0.84 | Neg 1 | 0.83 |
| A43034H | 0.04 | A43034H | 0.02 |
| A43045Hi | 0.00 | A43045Hi | 0.03 |
| A43081Hi | 0.06 | A43081Hi | 0.18 |

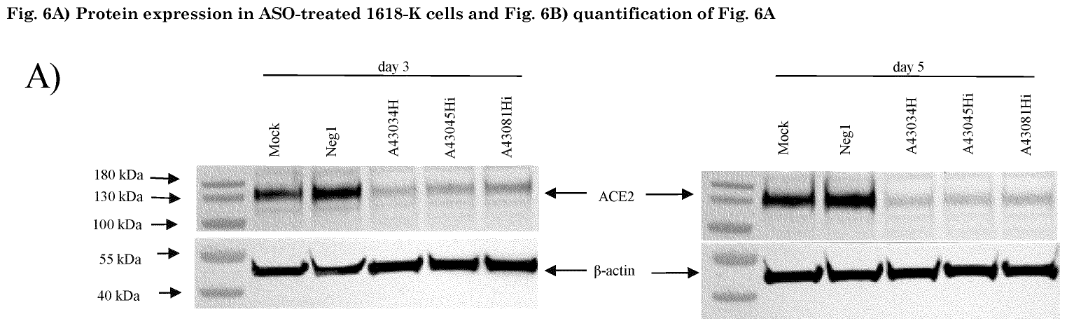

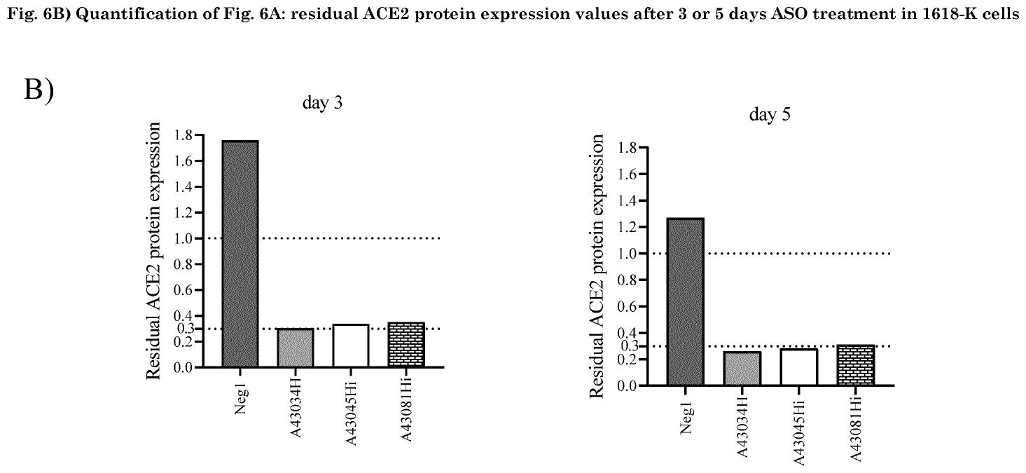

In order to investigate the knockdown efficacy of selected ACE2-specific ASOs at the protein level, 1618-K were treated with A43034H, A43045Hi and A43081Hi and the control oligonucleotide Neg1 at a concentration of 5μM for 3 or 5 days. For 5 days treatment, cells received additional ASO treatment at day 3 and were cultured for another 2 days. After 3 or 5 days treatment, the cells were lyzed. ACE2 protein expression was analyzed by Western Blot (iBlot 2 Dry Blotting System, ThermoFisher) and representative images are shown in FIG. 6A. Treatment with the different ACE2-specific ASOs for 3 or 5 days strongly reduced ACE2 protein expression as compared to mock-treated cells, whereas treatment with Neg1 had no negative impact on ACE2 protein expression after 3 or 5 days treatment in 1618-K cells (FIG. 6A). iBright Imager software was used for quantification of ACE2 and β-actin bands. Residual ACE2 expression is calculated as ACE2 band intensity compared to mock-treated cells (set as 1) normalized to the β-actin band intensity compared to mock-treated cells (set as 1) and depicted in FIG. 6B and Table 12. ASO treatment for 3 days reduced ACE2 protein expression by 69.61% (A43034H (SEQ ID NO.31), residual ACE2 expression of 0.30), 66.06% (A43045Hi (SEQ ID NO.42), residual ACE2 expression of 0.34) and 64.90% (A43081Hi (SEQ ID NO.78), residual ACE2 expression of 0.35) as compared to mock-treated cells (FIG. 6B and Table 12). 5 days treatment reduced ACE2 protein expression by 73.97% (A43034H (SEQ ID NO.31), residual ACE2 expression of 0.26), 71.85% (A43045Hi (SEQ ID NO.42), residual ACE2 expression of 0.28) and 68.65% (A43081Hi (SEQ ID NO.78), residual ACE2 expression of 0.31) as compared to mock-treated cells in 1618-K cells (FIG. 6B and Table 12).

TABLE 12
List of the mean Residual ACE2 protein expression values shown
as ACE2 band intensity compared to mock-treated cells normalized
to β-actin band intensity compared to mock-treated cells
(set as 1) in 1618-K cells after 3 or 5 days ASO treatment.

| | |
|---|---|
| | Residual ACE2 protein |
| | expression compared to mock- |
| ASO ID | treated cells (set as 1) |

3 days treatment

| | |
|---|---|
| Neg1 | 1.76 |
| A43034H | 0.30 |
| A43045Hi | 0.34 |
| A43081Hi | 0.35 |

5 days treatment

| | |
|---|---|
| Neg1 | 1.27 |
| A43034H | 0.26 |
| A43045Hi | 0.28 |
| A43081Hi | 0.31 |

### Example 6: Investigation of ACE2 mRNA Knockdown in Nasal Epithelial Cells Treated with ACE2-Specific ASOs

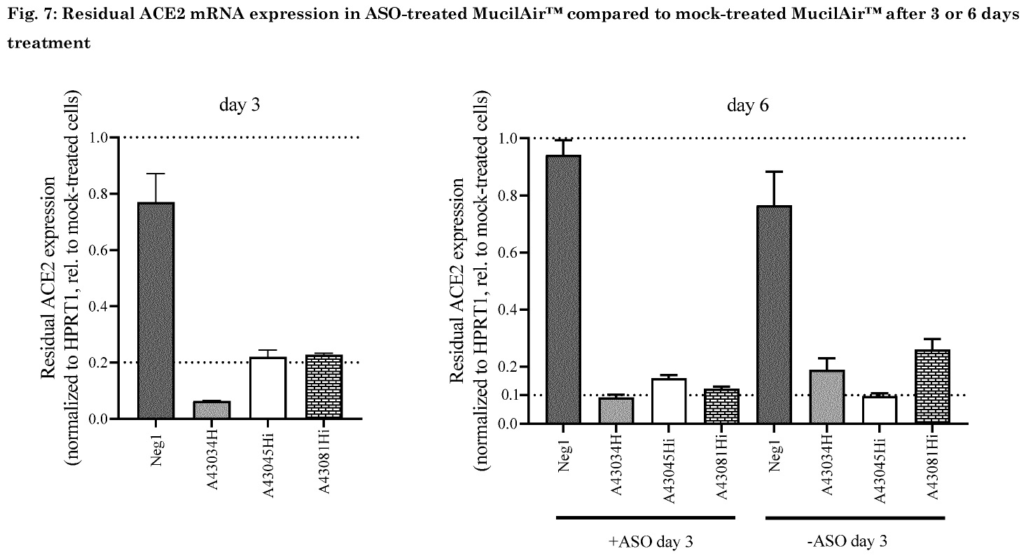

To test the knockdown efficacy of ACE2-specific ASOs in relevant cells of the upper airways, fully differentiated nasal epithelial cells cultured at the air liquid interface (MucilAir™, Epithelix) were treated at the apical and basal surface with the ACE2-specific ASOs A43034H (SEQ ID NO.31), A43045Hi (SEQ ID NO.42) and A43081Hi (SEQ ID NO.78) or the control oligonucleotide Neg1 at a concentration of 10 μM for 3 or 6 days. For 6 days treatment, MucilAir™ received additional ASO treatment at day 3 and were cultured for further 3 days (+ASO day 3). Furthermore, a condition was included where cells did not receive additional ASO on day 3 (−ASO day 3) and the cell culture medium was replaced by fresh medium without ASO. 3 or 6 days after starting the treatment, the cells were lyzed. ACE2 and HPRT1 mRNA expression was analyzed using the QuantiGene Singleplex assay (ThermoFisher) and the ACE2 expression values were normalized to HPRT1 values. The results for the ASO treatment are shown as residual ACE2 mRNA expression relative to mock-treated cells (set as 1) in FIG. 7 and Table 13. 3 days treatment with ACE2-specific ASOs reduced ACE2 mRNA expression by 93.67% (A43034H (SEQ ID NO.31), residual ACE2 mRNA expression of 0.06), 78.04% (A43045Hi (SEQ ID NO.42), residual ACE2 mRNA expression of 0.22) and 77.33% (A43081Hi (SEQ ID NO.78), residual ACE2 mRNA expression of 0.23) as compared to mock-treated cells in MucilAir™ (FIG. 7 and Table 13). Treatment of MucilAir™ with ASOs for a total treatment duration of 6 days resulted in the reduction of ACE2 mRNA expression by 90.77% (A43034H (SEQ ID NO.31), residual ACE2 mRNA expression of 0.09), 84.01% (A43045Hi (SEQ ID NO.42), residual ACE2 mRNA expression of 0.16) and 87.76% (A43081Hi (SEQ ID NO.78), residual ACE2 mRNA expression of 0.12) as compared to mock-treated cells (FIG. 7 and Table 13). ACE2 mRNA expression 6 days after start of treatment was reduced in MucilAir™ in which ASOs were removed on day 3 as shown by a reduction by 81.16% (A43034H (SEQ ID NO.31), residual ACE2 mRNA expression of 0.19), 90.28% (A43041Hi (SEQ ID NO.42), residual ACE2 mRNA expression of 0.10) and 74.00% (A43081Hi (SEQ ID NO.78), residual ACE2 mRNA expression of 0.26) as compared to mock-treated cells (FIG. 7 and Table 13).

TABLE 13
List of the mean ACE2 mRNA expression values in ASO-
treated MucilAir ™ compared to mock-treated
MucilAir ™ after 3 or 6 days ASO treatment.
Expression values are normalized to HPRT1.

| | |
|---|---|
| | Residual ACE2 expression |
| | compared to mock-treated cells |
| ASO ID | (set as 1) |

3 days treatment

| | |
|---|---|
| Neg1 | 0.77 |
| A43034H | 0.06 |
| A43045Hi | 0.22 |
| A43081Hi | 0.23 |

6 days treatment

| | |
|---|---|
| Neg1 + ASO d3 | 0.94 |
| A43034H + ASO d3 | 0.09 |
| A43045Hi + ASO d3 | 0.16 |
| A43081Hi + ASO d3 | 0.12 |
| Neg1 − ASO d3 | 0.77 |
| A43034H − ASO d3 | 0.19 |
| A43045Hi − ASO d3 | 0.10 |
| A43081Hi − ASO d3 | 0.26 |

### Example 7: Inhibition of SARS-CoV-2 Virus Replication in HEK-293T Cells Treated with ACE2-Specific ASOs

ACE2-specific ASOs of the present invention, amongst others A43034H (SEQ ID NO.31), A43045Hi (SEQ ID NO.42) and A43081Hi (SEQ ID NO.78), were administered to HEK-293T cells. Cells were grown for 5 days and every 3 days 5μM of the oligonucleotide of the present invention were added to the cell culture.

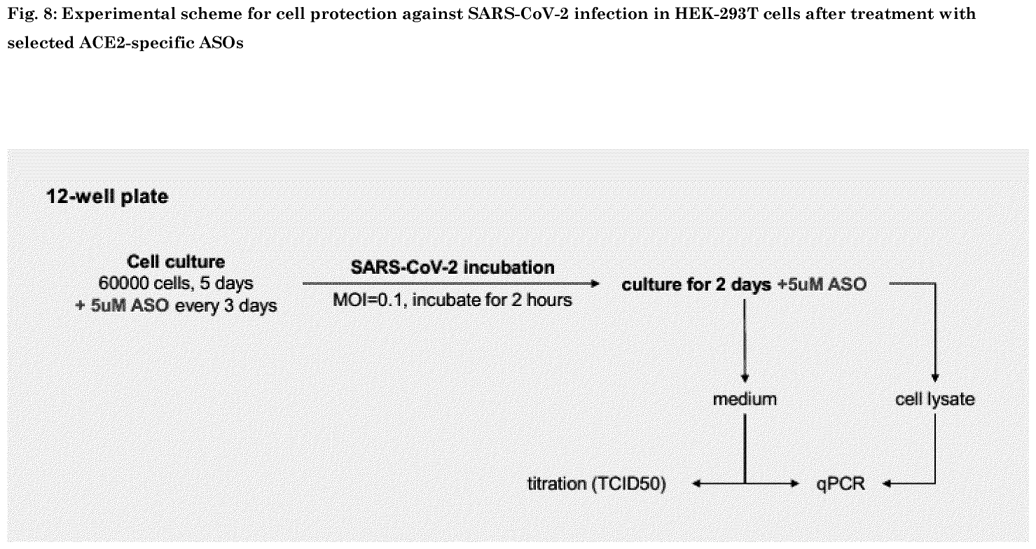

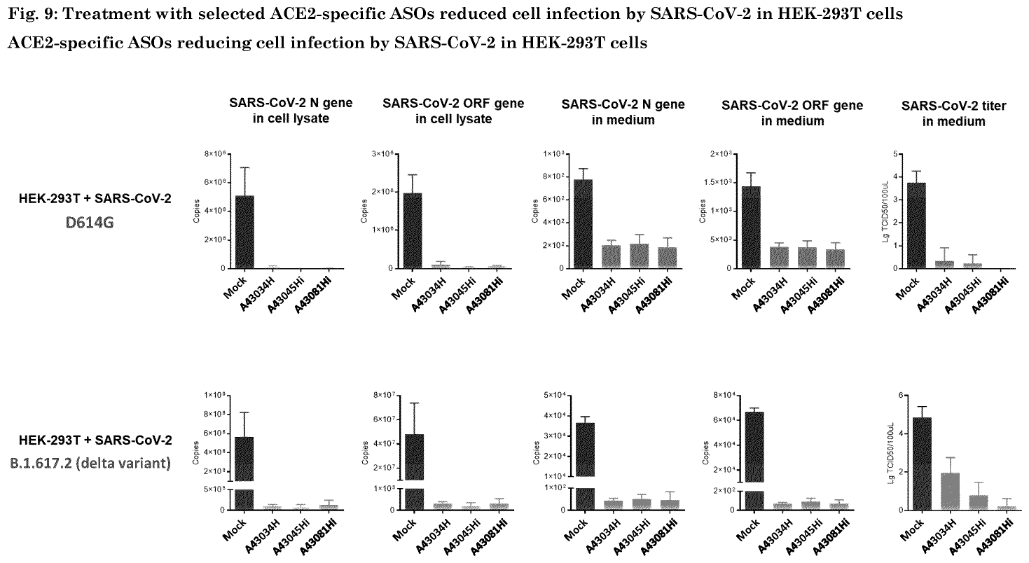

Afterwards the cells were incubated with severe acute respiratory syndrome coronavirus 2 (SARS-CoV-2) for 2 h (MOI=0.1) and subsequently 5 μM of an ACE2-specific ASO of the present invention was added for two days. Afterwards the cells were harvested and the medium and the cell lysate were investigated separately via quantitative (q) PCR directed to SARS-CoV-2 N gene and SARS-CoV-2 ORF gene, and the medium additionally via titration (TCID₅₀). The treatment scheme is shown in FIG. 8. The results of this experiment are shown in FIG. 9 and Table 14. The ACE2-specific ASOs of the present invention protect HEK-293T cells from SARS-CoV-2 infection by more than 2 orders of magnitude. Different variants of the SARS-CoV-2 were tested which were the variants D614G and B.1.617.2 (delta variant). Treatment of HEK-293T cells with ACE2-specific ASOs of the present invention led to reduced SARS-CoV2 gene expression as well as to strongly reduced viral titers compared to untreated cells. Inhibition of virus gene expression was even more pronounced in cells infected with B.1.617.2 (delta variant), while virus titers were similarly affected independent of the used virus variant (FIG. 9).

TABLE 14
List of the mean SARS-Cov-2 N gene, ORF gene in cell lysate and medium
after treatment with ACE2-specific ASOs and infection with SARs-
CoV-2 D614G and B.1.617.2 (delta variant) in HEK-293T cells.

| | | | | | |
|---|---|---|---|---|---|
| | N_gene | ORF_gene | N_gene | ORF_gene | SARS-CoV-2 |
| | copies in | copies in | copies in | copies in | titer in medium |
| ASO ID | cell lysate | cell lysate | medium | medium | (LgTCID50/100 uL) |

Detected virus in HEK-293T 2 days after SARS-CoV-2 infection (SARS-CoV-2 D614G)

| | | | | | |
|---|---|---|---|---|---|
| Mock | 5061357 | 1970944 | 777 | 1434 | 3.75 |
| A43034H | 103285 | 100396 | 204 | 376 | 0.33 |
| A43045Hi | 43023 | 37681 | 217 | 368 | 0.22 |
| A43081Hi | 70760 | 61465 | 187 | 334 | 0.00 |

Detected virus in HEK-293T 2 days after SARS-CoV-2 infection (SARS-CoV-2 B.1.617.2)

| | | | | | |
|---|---|---|---|---|---|
| Mock | 566197856 | 48157647 | 36636 | 66743 | 4.84 |
| A43034H | 94867 | 31343 | 44 | 68 | 1.94 |
| A43045Hi | 75113 | 17493 | 52 | 90 | 0.78 |
| A43081Hi | 133912 | 31734 | 45 | 68 | 0.22 |

### Example 8: Inhibition of ACE2 Expression in Human Nasal Epithelial Cells (hNEC) after Treatment with ACE2-Specific ASOs

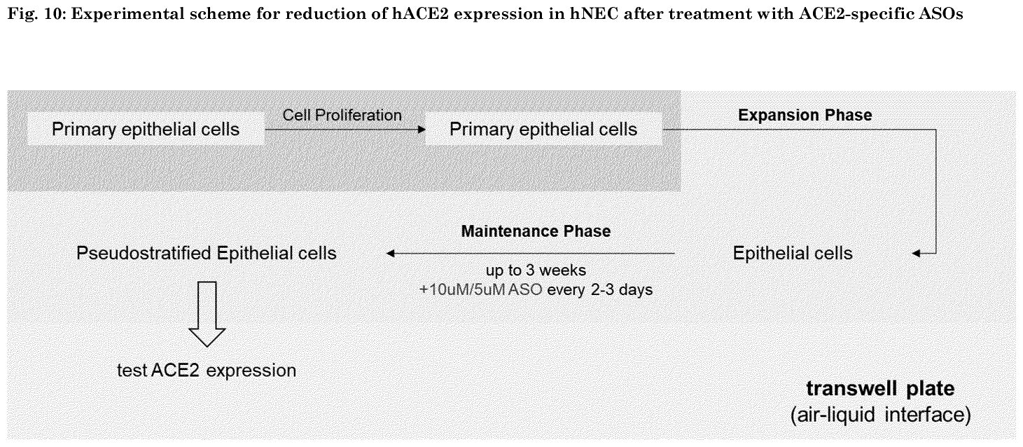

Primary hNEC were collected from human nasal mucosa and proliferated. Then hNEC were transferred onto transwell inserts in Pneumacult Ex medium (expansion phase). Once confluent, PneumaCult™-ALI Medium was added and hNEC grew on an air-liquid interface. During this maintenance phase 10 μM or 5 μM of an ACE2-specific ASO such as A43034H (SEQ ID NO.31), A43045Hi (SEQ ID NO.42) or A43081Hi (SEQ ID NO.78) were added to the hNEC every 2 to 3 days. After 3 weeks of cell culture, the hNEC differentiated into pseudostratified epithelial cells. These cells were lyzed and the ACE2 protein expression was determined by Western Blot. The treatment scheme is shown in FIG. 10.

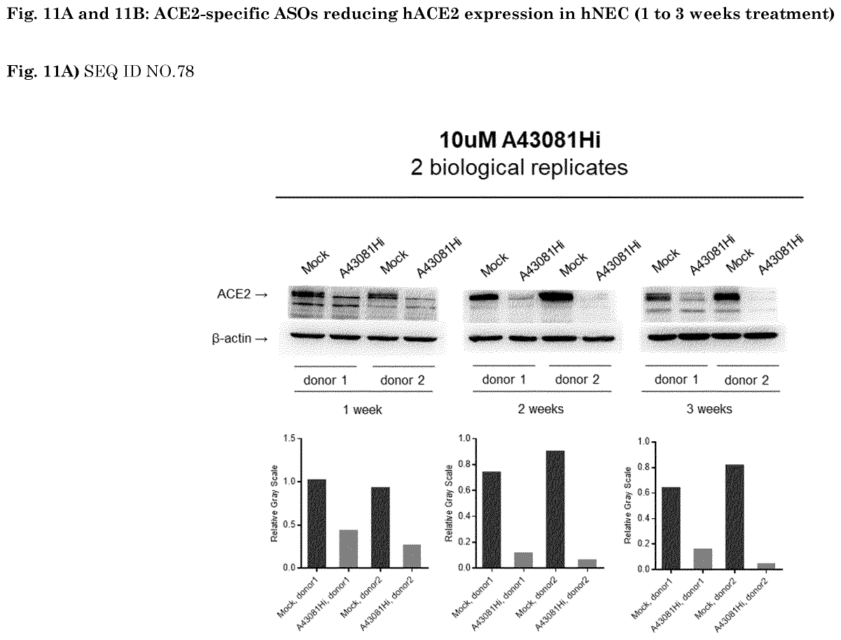

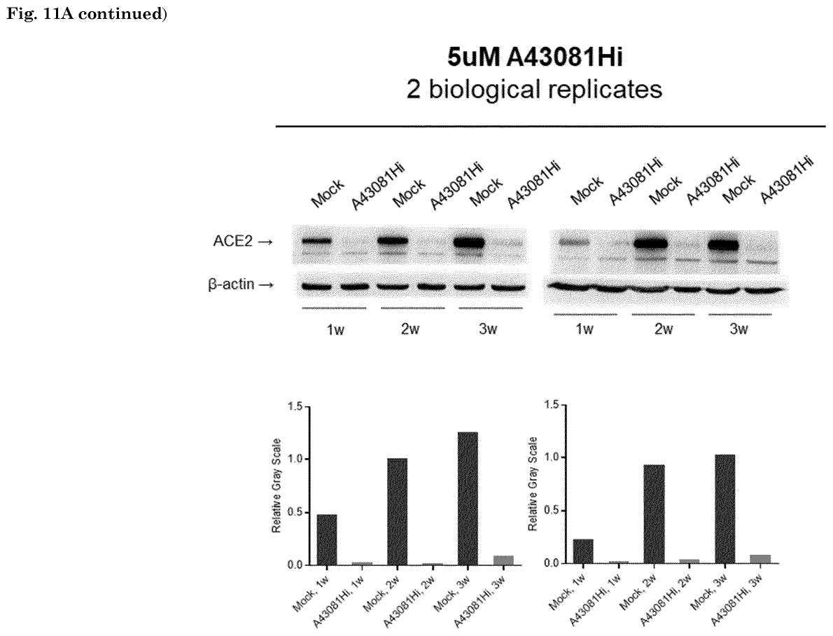

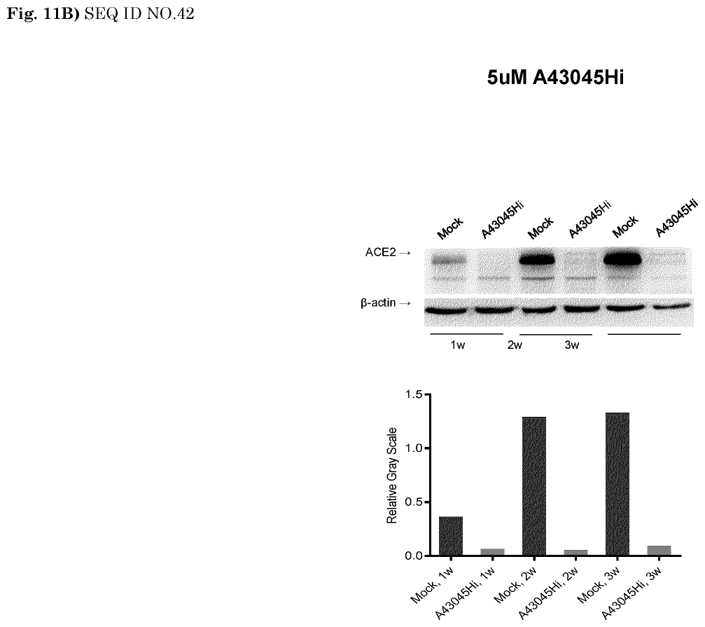

FIGS. 11A and 11B show the results of this experiment during 1 to 3 weeks of treatment of the hNEC with ACE2-specific ASOs of the present invention: the ACE2 protein expression in hNEC is significantly decreased after ASO treatment as compared to mock-treated cells. In FIG. 11A hNEC were treated with 10 μM or 51 μM A43081Hi (SEQ ID NO.78), in FIG. 11B hNEC were treated with 51 μM A43045Hi (SEQ ID NO.42).

Imager software was used for quantification of ACE2 and β-actin bands. Residual ACE2 expression is calculated as ACE2 band intensity compared to mock-treated cells (set as 1) normalized to the β-actin band intensity compared to mock-treated cells (set as 1) and depicted in FIGS. 11A and 11B and Table 15. 10 μM ASO treatment for 1 week, 2 weeks and 3 weeks reduced ACE2 protein expression by 63%, 87% and 83% (A43081Hi (SEQ ID NO.78), residual ACE2 expression of 0.37; 0.13 and 0.17 respectively (FIG. 11A). 5 μM ASO treatment for 1 week, 2 weeks and 3 weeks reduced ACE2 protein expression by 88%, 95% and 91% (A43081Hi (SEQ ID NO.78), residual ACE2 expression of 0.12; 0.05 and 0.09 respectively (FIG. 11A). 5 μM ASO treatment for 1 week, 2 weeks and 3 weeks reduced ACE2 protein expression by 78%, 94% and 92% (A43045Hi (SEQ ID NO.42), residual ACE2 expression of 0.22; 0.06 and 0.08 respectively (FIG. 11B)

TABLE 15
List of the mean Residual ACE2 protein expression values shown
as ACE2 band intensity compared to mock-treated cells normalized
to β-actin band intensity compared to mock-treated cells
(set as 1) in hNECs after 1 to 3 weeks of ASO treatment.

| | |
|---|---|
| | Residual ACE2 protein expression |
| Time of ASO treatment | compared to mock-treated cells (set as 1 |

10 μM A43081Hi treatment

| | |
|---|---|
| 1 week | 0.37 |
| 2 weeks | 0.13 |
| 3 weeks | 0.17 |

5 μM A43081Hi treatment

| | |
|---|---|
| 1 week | 0.12 |
| 2 weeks | 0.05 |
| 3 weeks | 0.09 |

5 μM A43045Hi treatment

| | |
|---|---|
| 1 week | 0.22 |
| 2 weeks | 0.06 |
| 3 weeks | 0.08 |

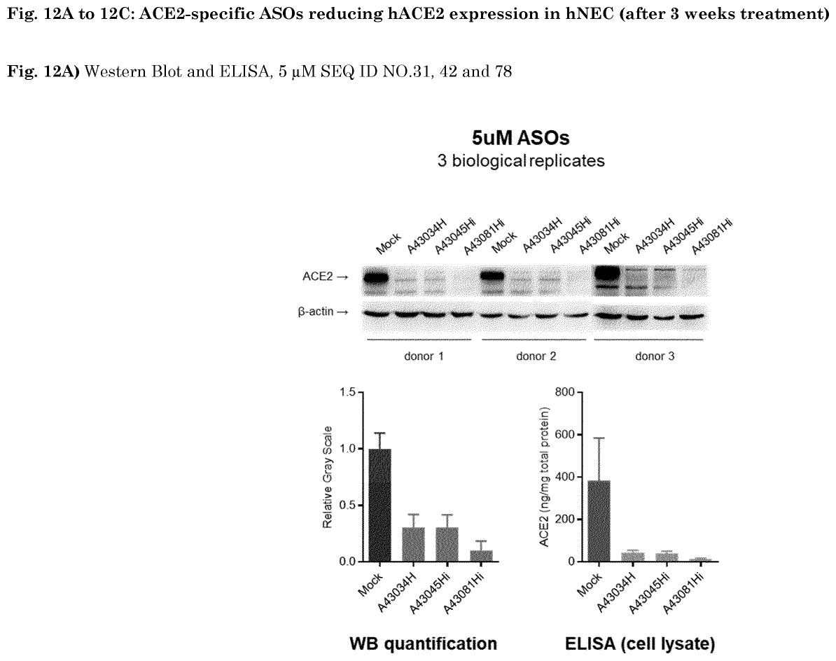

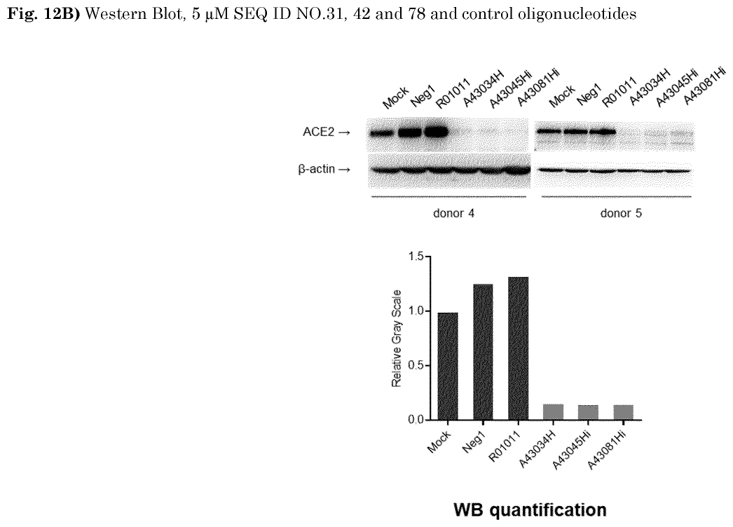

FIGS. 12A to 12C show ACE2 expression of hNEC after 3 weeks of treatment with 5 μM of an oligonucleotide of the present invention, i.e., 5 μM of A43034H (SEQ ID NO.31), A43045Hi (SEQ ID NO.42) and A43081Hi (SEQ ID NO.78), respectively. FIG. 12A shows the expression of ACE2 on the protein level in a Western Blot and an ELISA prepared from the cell lysate. FIG. 12B shows a Western Blot where the effect of a control oligonucleotide (Neg1, R01011) has been tested in comparison to A43034H, A43045Hi and A43081Hi. Neg1 and R01011 are control oligonucleotides that do not have sequence complementarity to any human gene. Treatment with the different ACE2-specific ASOs for 3 weeks strongly reduced ACE2 protein expression as compared to mock-treated cells (FIG. 12A and 12B), whereas treatment with control oligonucleotide Neg1 or R01011 had no negative impact on ACE2 protein expression in hNEC (FIG. 12B). ACE2 protein expression was quantified by Western Blot and ELISA in mock and ASO-treated cells and depicted in FIG. 12A. FIG. 12B and Table 16.

ASO treatment reduced ACE2 protein expression by 70%, 70% and 90% (A43034H (SEQ ID NO.31, A43045Hi (SEQ ID NO.42), A43081Hi (SEQ ID NO. 78), residual ACE2 expression of 0.30; 0.30 and 0.10 respectively (FIG. 12A).

ASO treatment reduced ACE2 protein expression by 84%, 85% and 85% (A43034H (SEQ ID NO.31), A43045Hi (SEQ ID NO.42), A43081Hi (SEQ ID NO.78), residual ACE2 expression of 0.16; 0.15 and 0.15 respectively (FIG. 12B). In contrast, treatment with the control oligonucleotides Neg1 or R01011 did not show any effect at the protein level (FIG. 12B).

TABLE 16
List of the mean ACE2 values measured in the cell lysate
of mock and ASO-treated cells after 3 weeks of treatment
in hNECs analyzed by Western Blot and ELISA.
WB quantification (3 weeks treatment)

| | Residual ACE2 protein expression |
|---|---|
| | compared to mock-treated cells (set |
| ASO ID | as 1) |
| A43034H | 0.30 |
| A43045Hi | 0.30 |
| A43081Hi | 0.10 |

ELISA (3 weeks treatment)

| | ACE2 protein expression |
|---|---|
| ASO ID | (ng/mg total protein) |
| Mock control | 381.53 |
| A43034H | 43.85 |
| A43045Hi | 39.85 |
| A43081Hi | 11.81 |

Western Blot (3 weeks treatment)

| | Residual ACE2 protein expression |
|---|---|
| | compared to mock-treated cells (set |
| ASO ID | as 1) |
| Neg1 | 1.26 |
| R01011 | 1.33 |
| A43034H | 0.16 |
| A43045Hi | 0.15 |
| A43081Hi | 0.15 |

### Example 9: Treatment with ACE2-Specific ASOs Protecting hNEC from SARS-CoV-2 Infection in Vitro

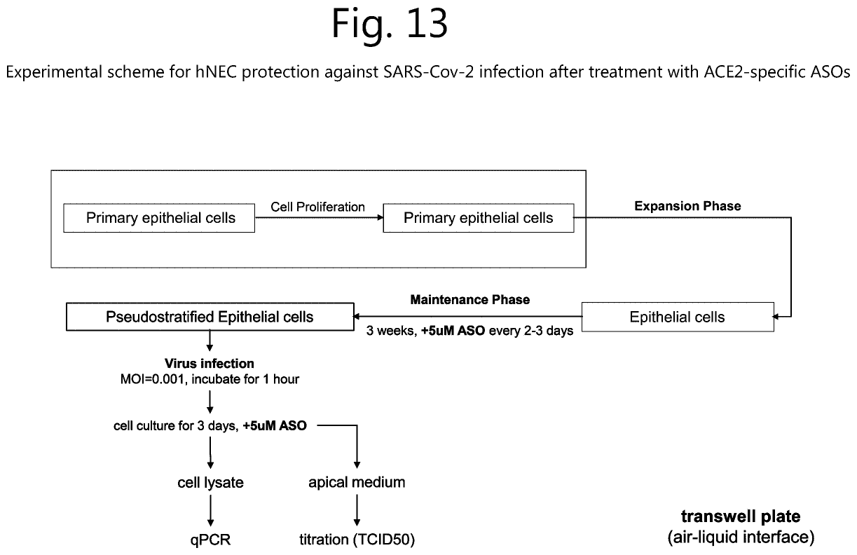

Primary hNEC were collected from human nasal mucosa and proliferated. Then hNEC were transferred onto transwell inserts in Pneumacult Ex medium (expansion phase). Once confluent, PneumaCult™-ALI Medium was added and hNEC grew on an air-liquid interface. During this maintenance phase 5 μM of an ACE2-specific ASO such as A43034H (SEQ ID NO.31), A43045Hi (SEQ ID NO.42) or A43081Hi (SEQ ID NO.78) were added to the hNEC every 2 to 3 days. After 3 weeks of cell culture, the cells differentiated into a pseudostratified phenotype. These cells were infected with SARS-CoV-2 (MOI=0.001) for 1 h. Afterwards the cells were kept for further 3 days and incubated with 5 μM of an ACE2-specific ASO of the present invention such as A43034H, A43045Hi or A43081Hi. After these three days apical medium and cell lysate were harvested. The amount of SARS-CoV-2 was determined in the cell lysate via qPCR directed to SARS-CoV-2 N gene and SARS-CoV-2 ORF gene, and in the apical medium via titration (TCID₅₀). A scheme of this experiment is shown in FIG. 13.

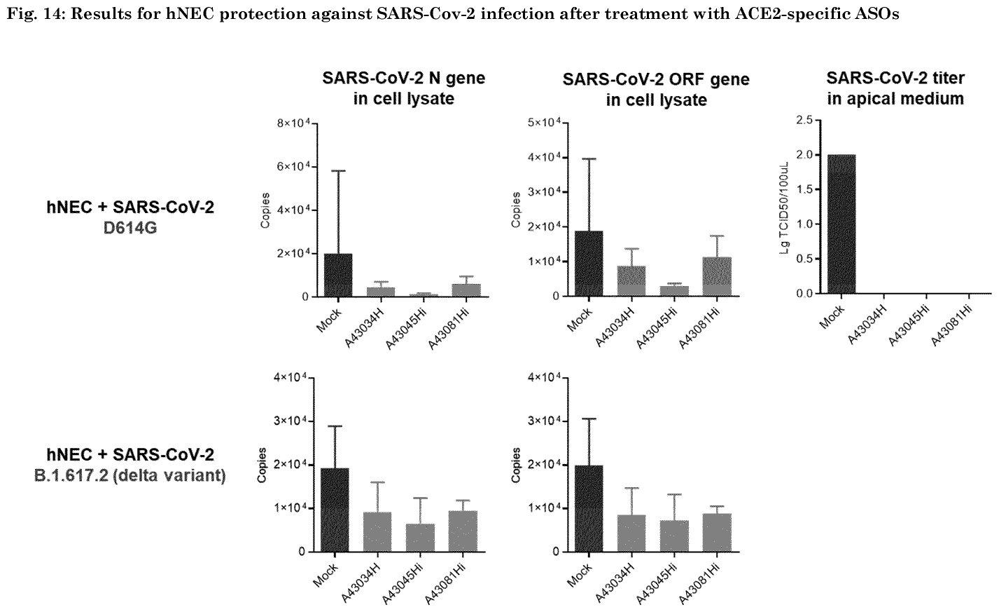

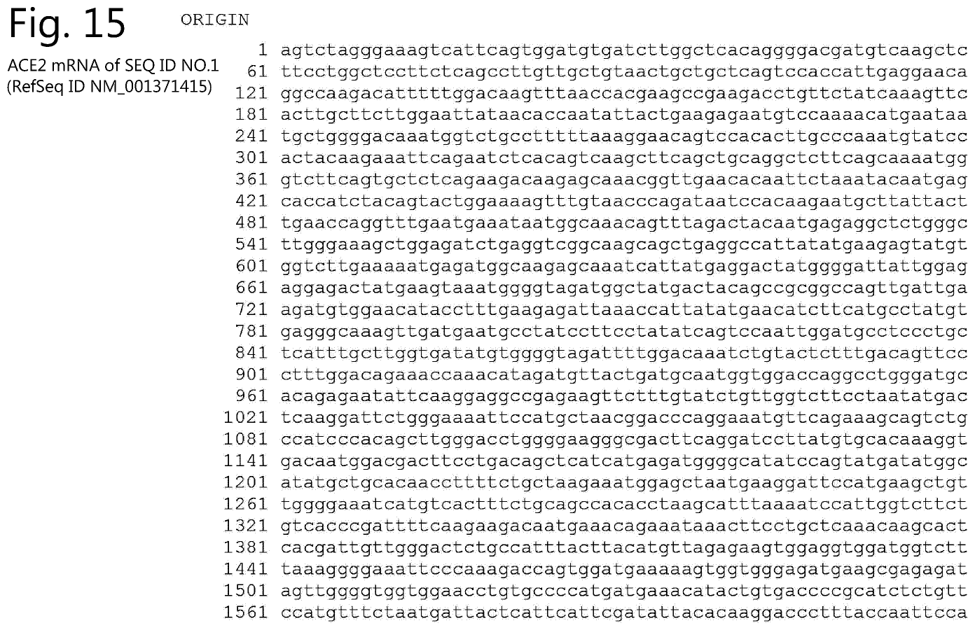

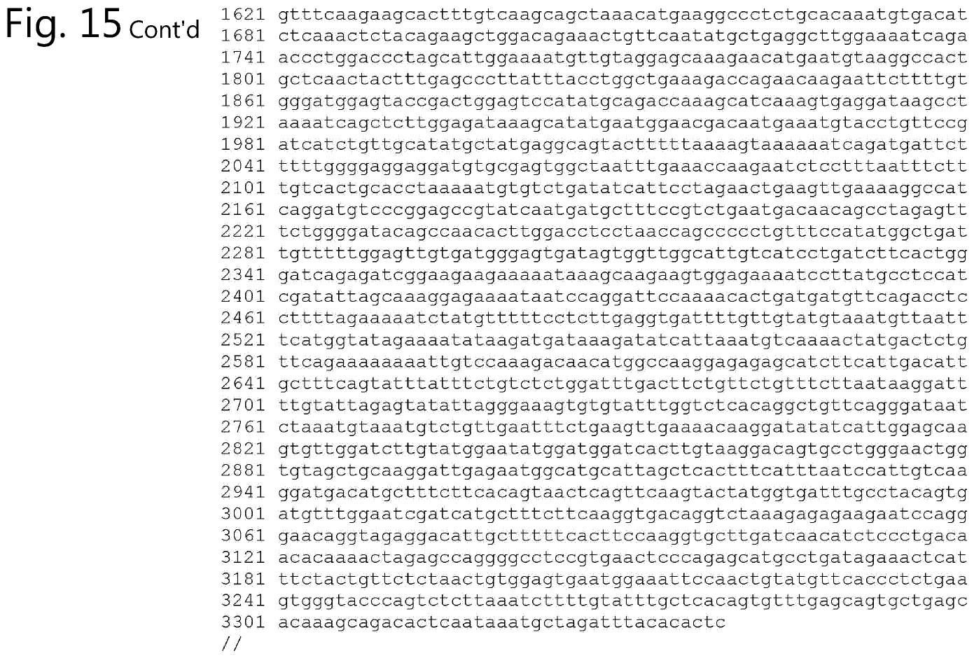

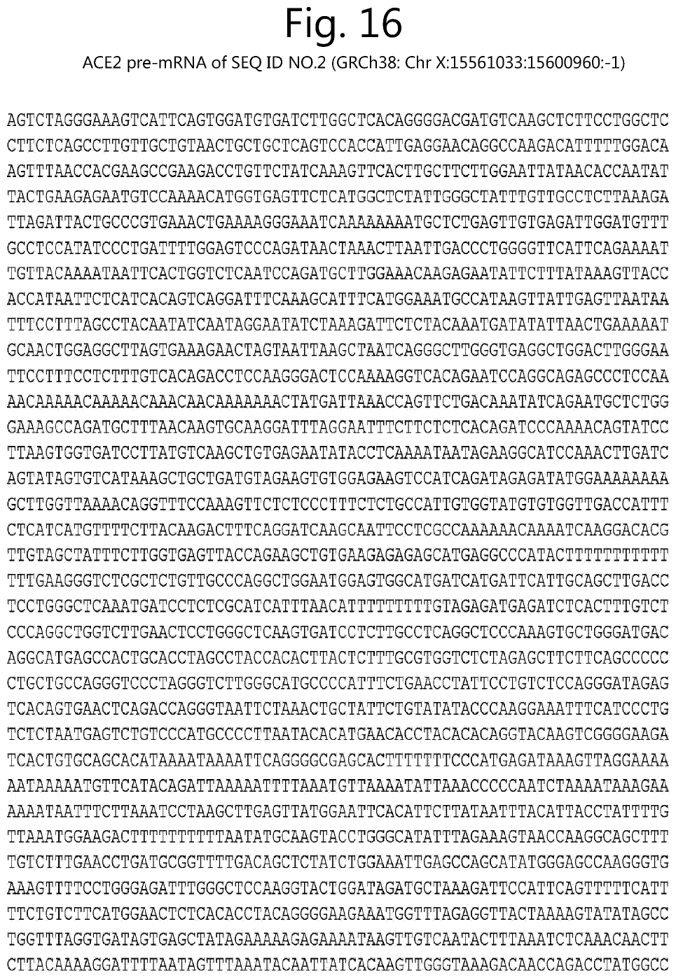

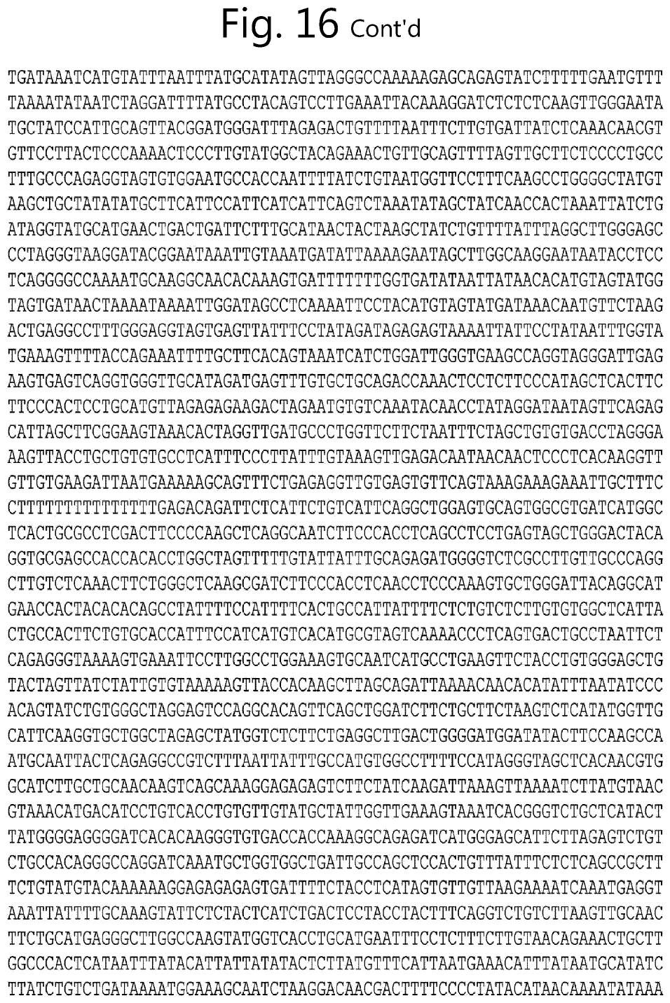

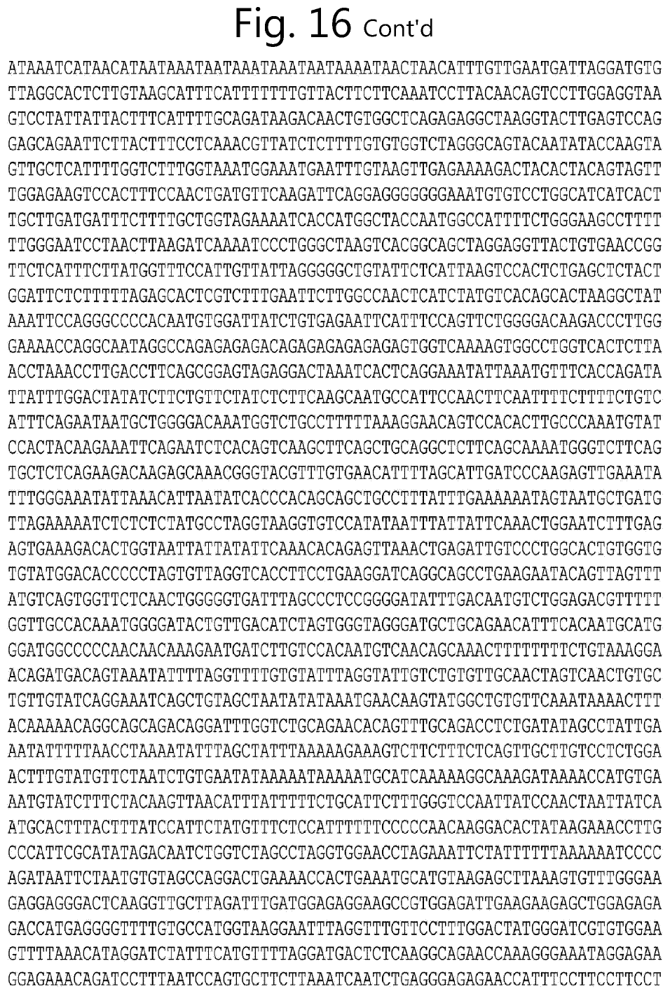

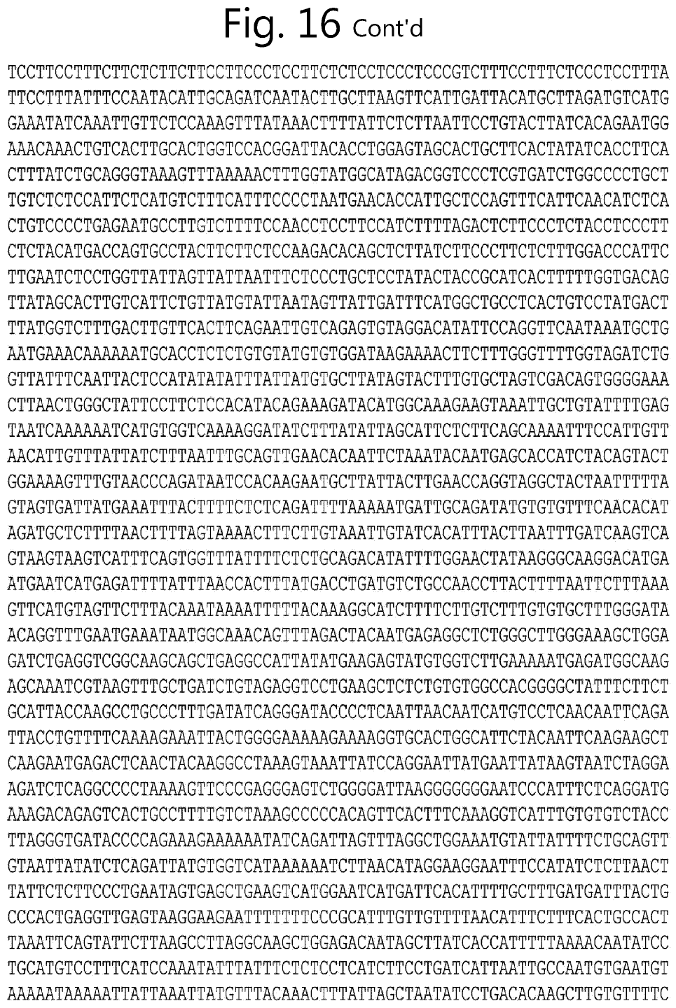

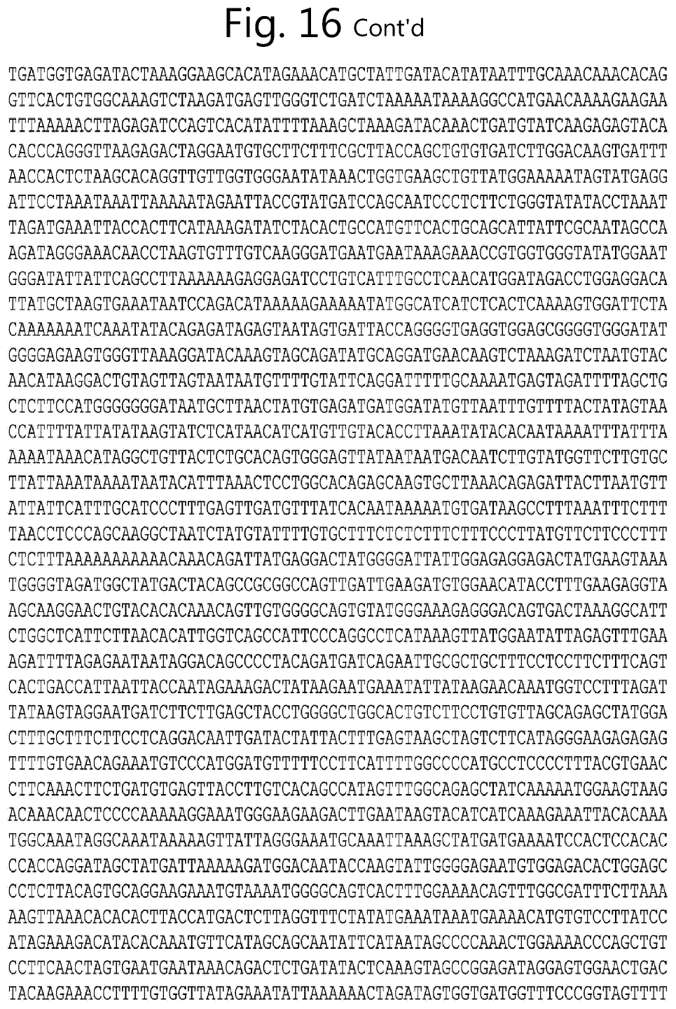

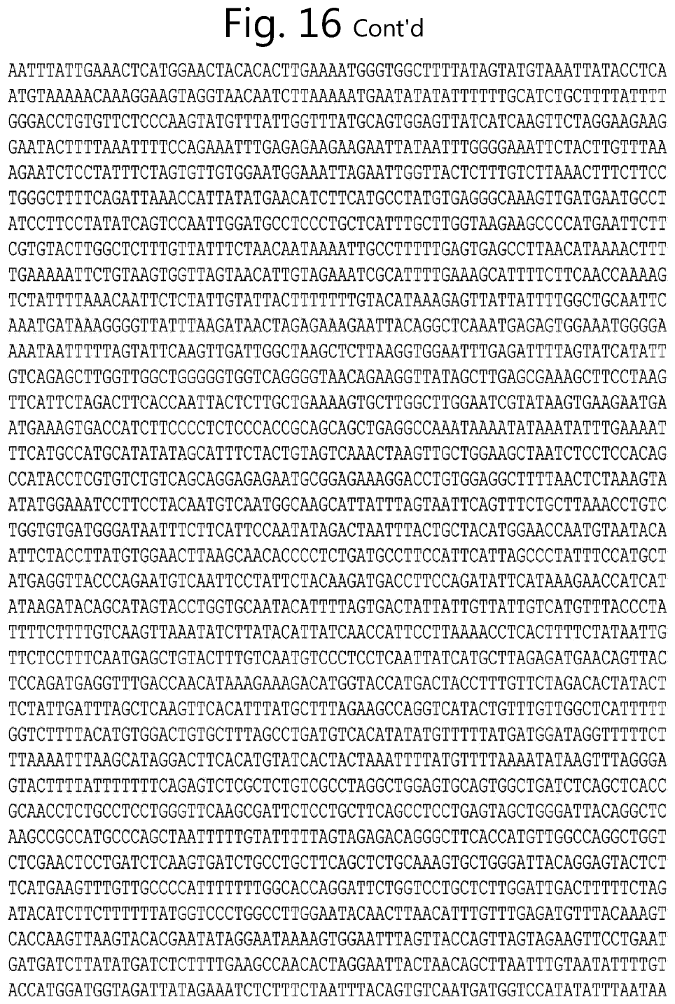

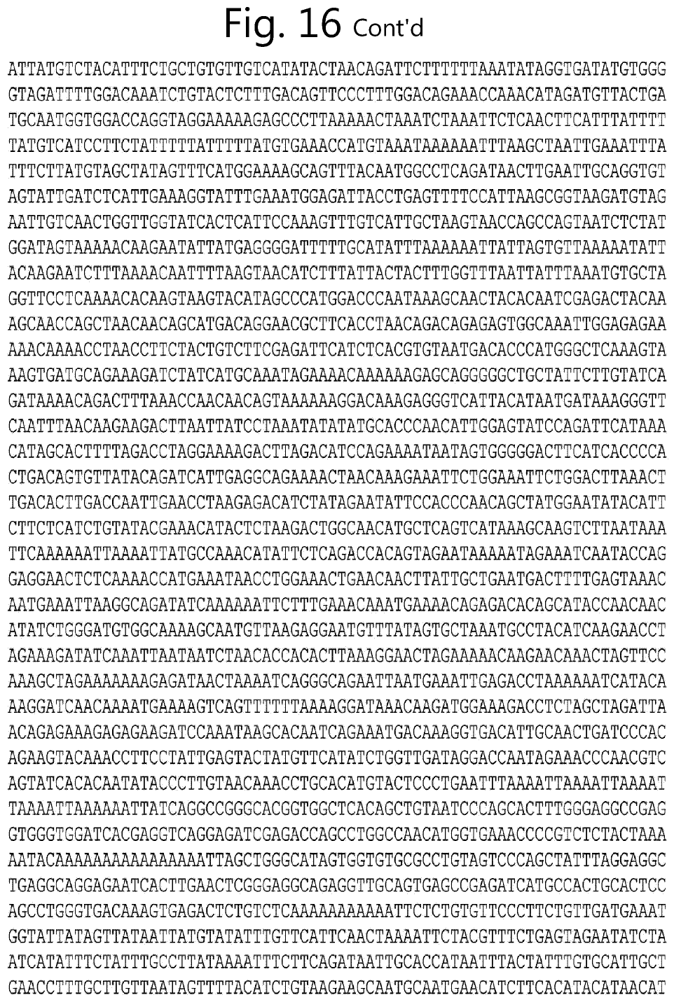

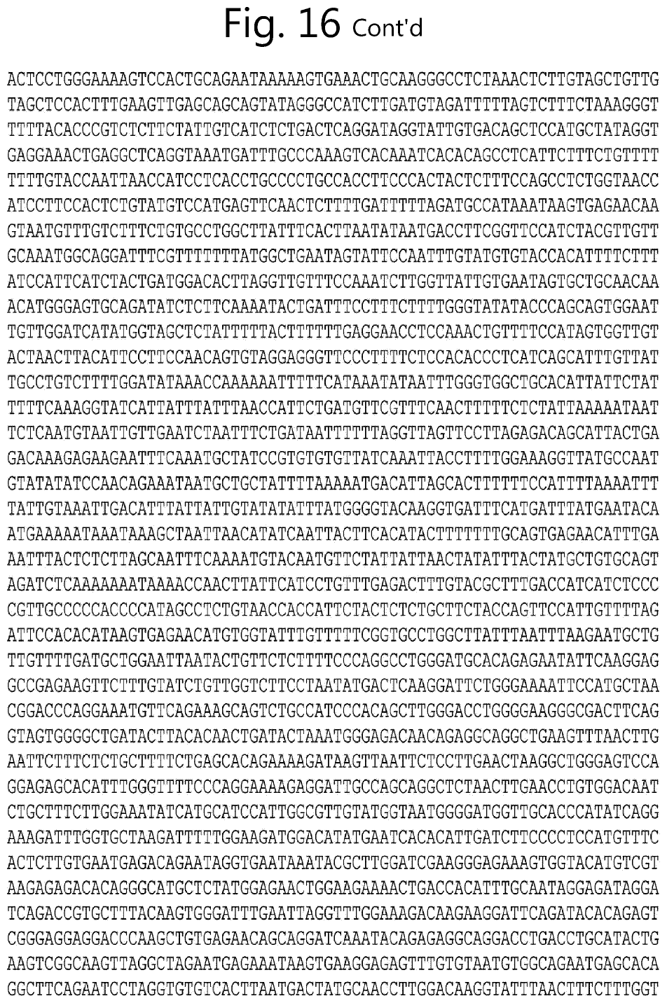

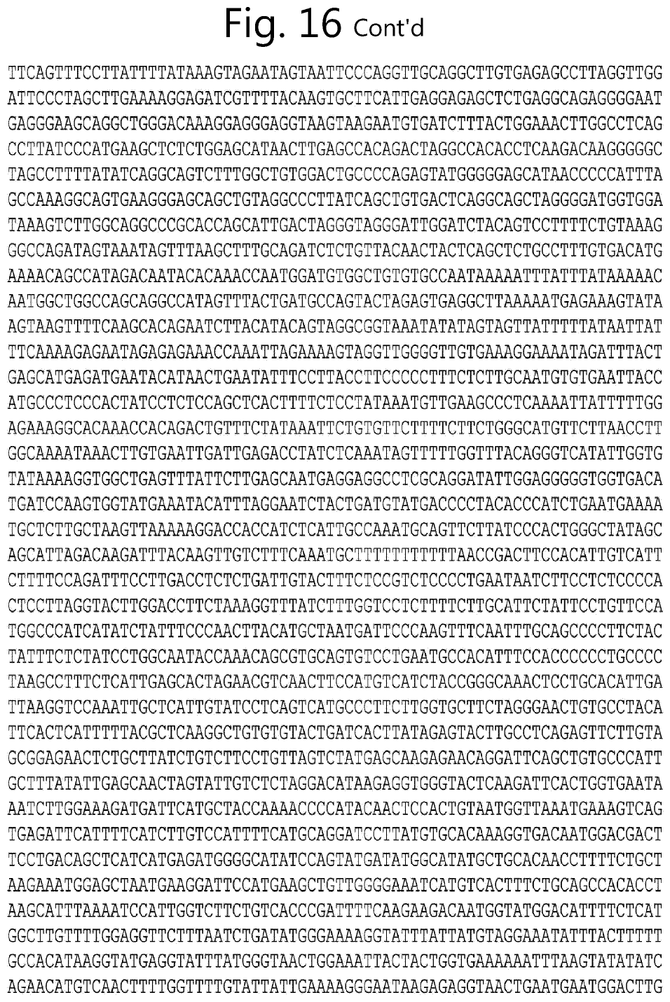

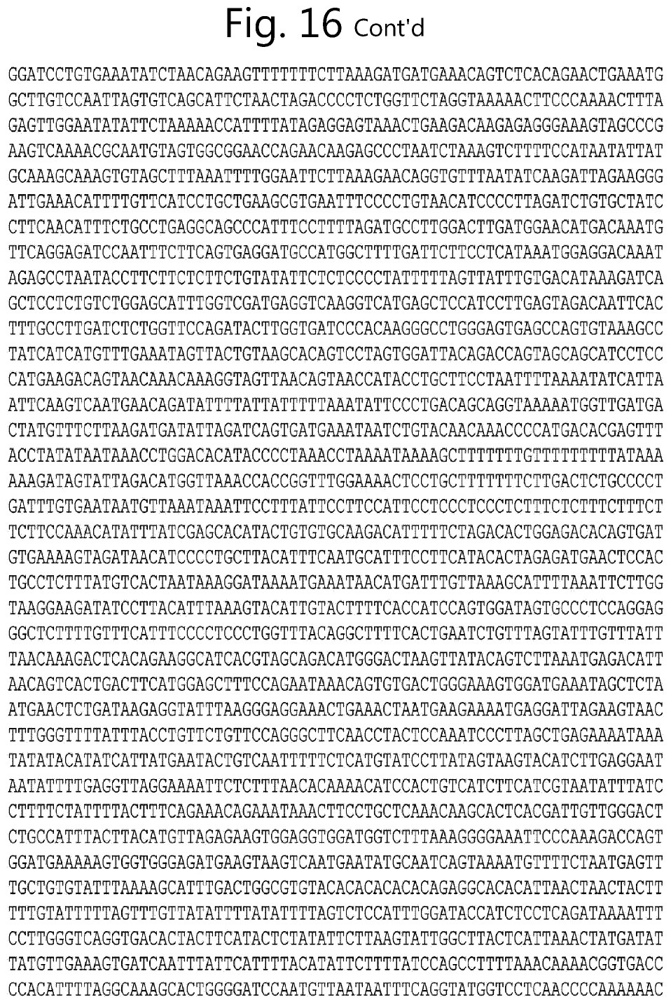

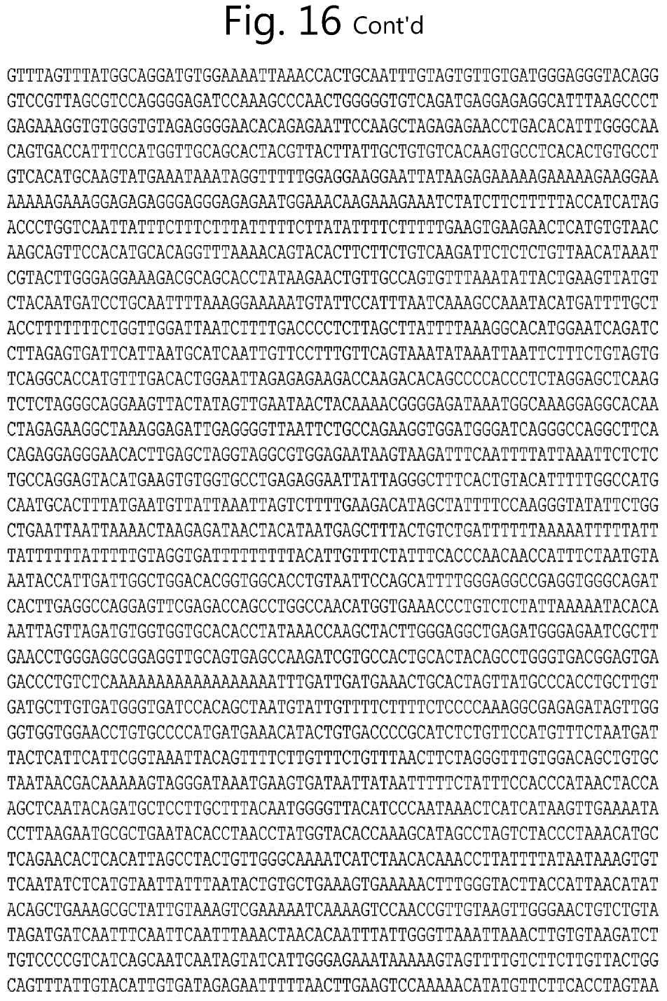

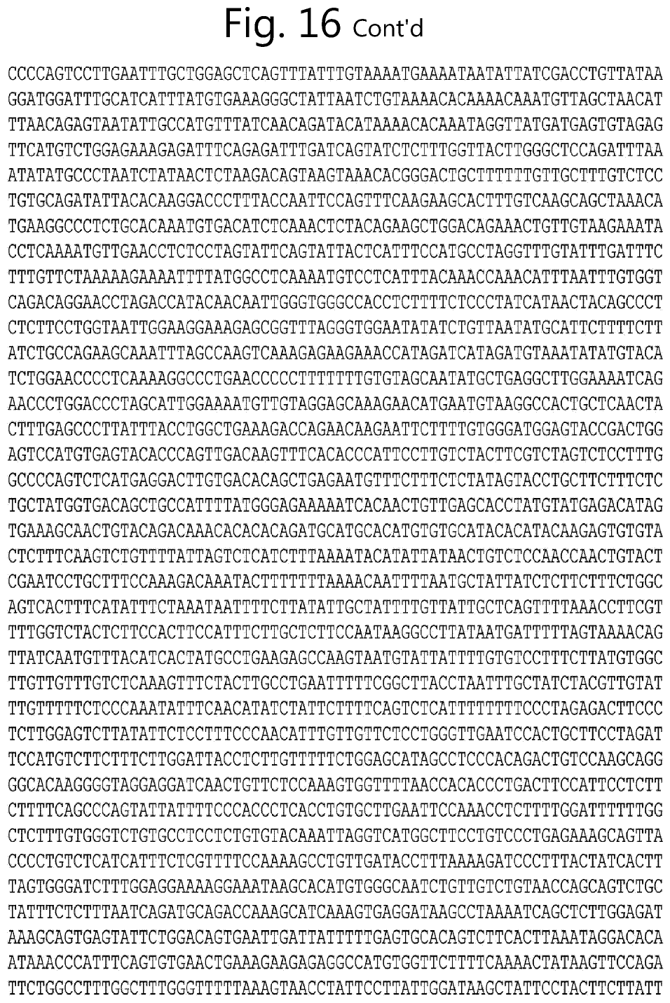

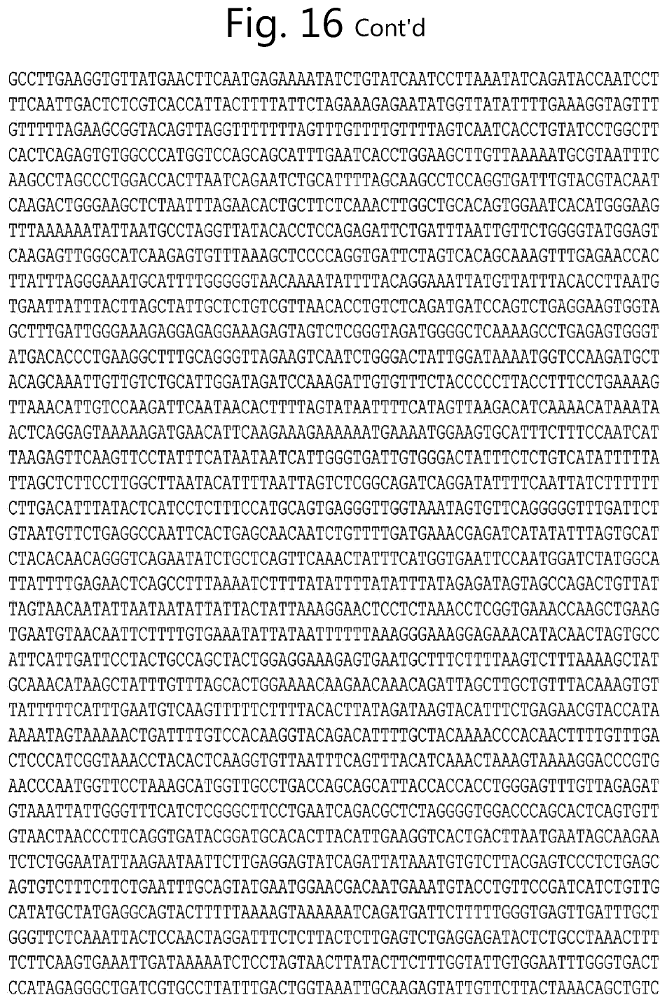

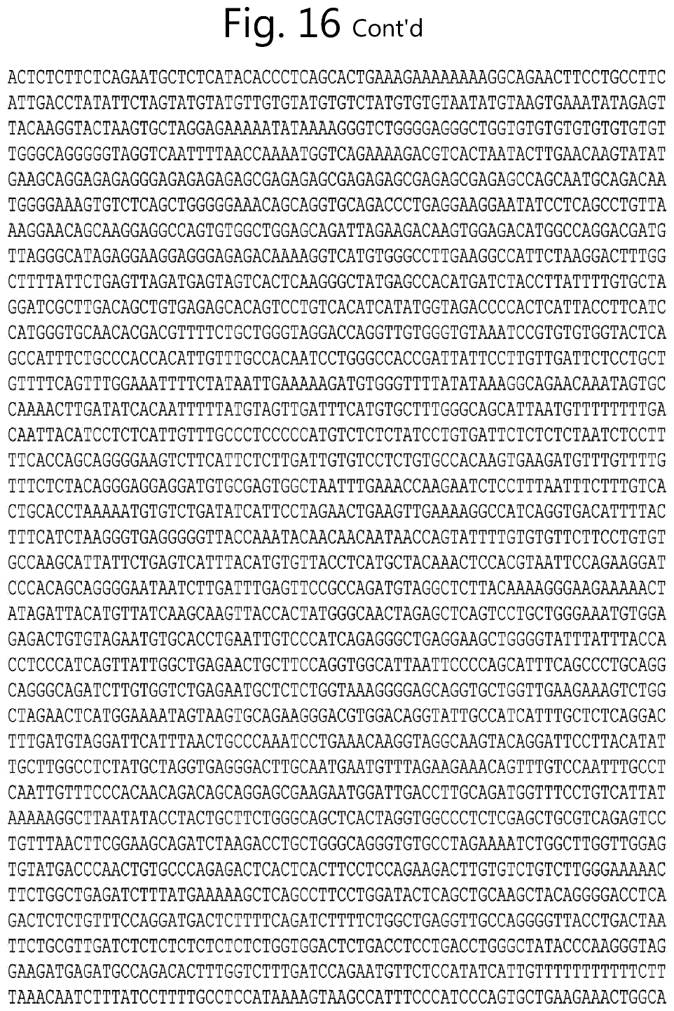

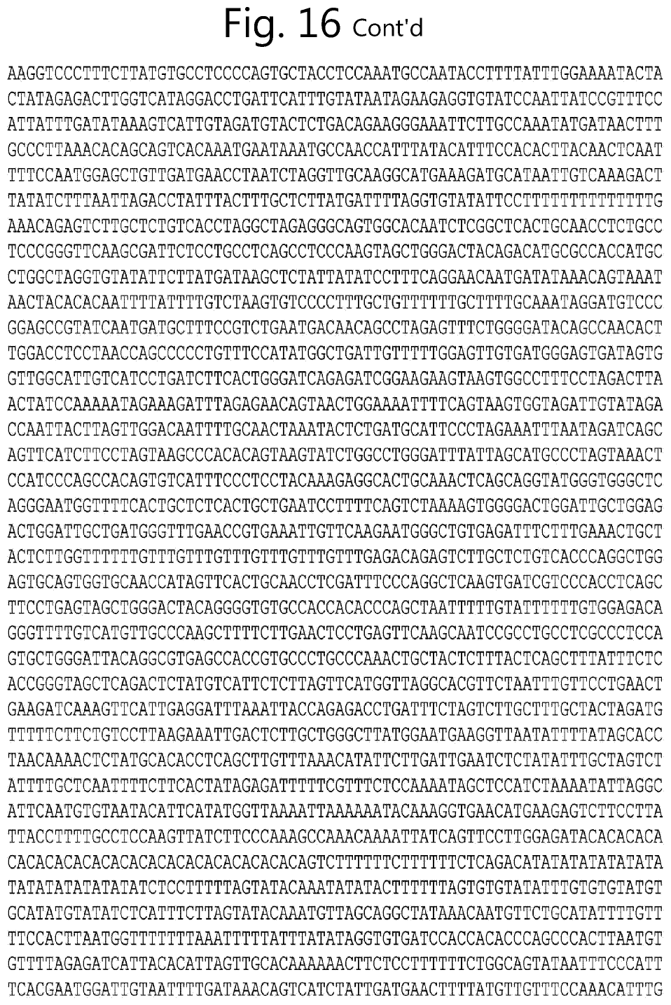

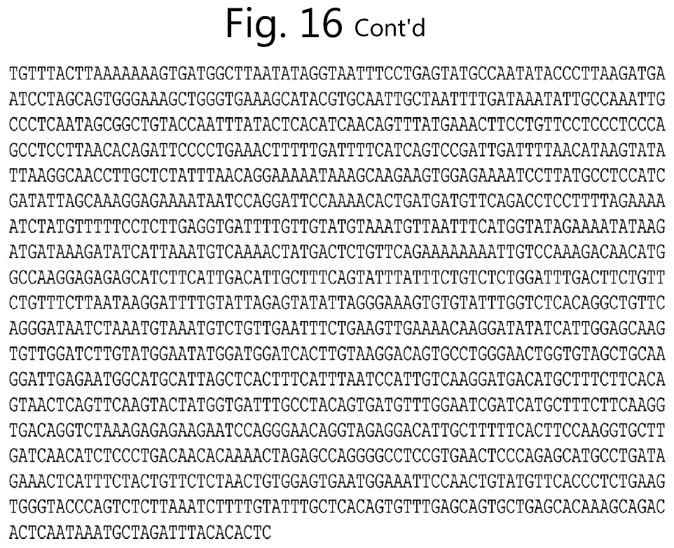

The results of this experiment of a SARS-CoV-2 infection of MOI=0.001 are shown in FIG. 14 and Table 17. Different variants of the SARS-CoV-2 were tested which were the variants D614G and B.1.617.2 (delta variant). Infection of cells treated with ACE2-specific ASO of the present invention showed reduced viral gene expression and SARS-CoV-2 propagation compared to untreated cells (mock) (FIG. 14).

TABLE 17
List of the mean SARS-Cov-2 N gene, ORF gene
in cell lysate and medium after treatment with
ACE2-specific ASOs and infection with SARs-CoV-2
D614G and delta variant in hNEC.
Detected virus in hNEC 3 days after SARS-CoV-2 infection

| | N_gene | ORF_gene | N_gene | ORF_gene | SARS-CoV-2 titer |
|---|---|---|---|---|---|
| | copies in | copies in | copies in | copies in | in apical medium |
| | cell lysate | cell lysate | cell lysate | cell lysate | (LgTCID50/100 μL) |
| ASO ID | (D614G) | (D614G) | (B.1.617.2) | (B.1.617.2) | (D614G) |
| Mock | 20038 | 18852 | 19323 | 19930 | 2 |
| A43034H | 4640 | 8732 | 9208 | 8558 | 0 |
| A43045Hi | 1380 | 2977 | 6542 | 7208 | 0 |
| A43081Hi | 6029 | 11259 | 9439 | 8791 | 0 |

1. Oligonucleotide comprising 10 to 25 nucleotides, wherein at least one nucleotide is modified, hybridizing with mRNA of angiotensin-converting enzyme 2 (ACE2) of SEQ ID NO.1 and/or with pre-mRNA of ACE2 of SEQ ID NO.2 resulting in a reduction of the level of ACE2, ACE2 mRNA, ACE2 pre-mRNA or a combination thereof of 30 to 99% compared to an untreated control.

2. Oligonucleotide according to claim 1, wherein the modification of the nucleotide is selected from the group consisting of a bridged nucleic acid such as LNA, ENA, a 2′Fluoro modified nucleotide, a 2 O-Methyl modified nucleotide, a 2 O-Methoxy modified nucleotide, a FANA and a combination thereof.

3. Oligonucleotide according to claim 1 or 2 hybridizing with ACE2 of SEQ ID NO.1 and/or SEQ ID NO.2, wherein the oligonucleotide hybridizes outside a hybridizing active region or within a hybridizing active region of position 28945 to 29744, position 22545 to 23344, position 39345 to 39928, position 9745 to 10544, position 12945 to 13744, position 34545 to 35344, position 36145 to 36944, position 38545 to 39344, position 24945 to 25744, position 36145 to 36944, position 19345 to 20144, position 14545 to 15344, position 30545 to 31344, position 24145 to 24944, position 16945 to 17744, position 145 to 944, position 21745 to 22544, position 20145 to 20944, position 37745 to 38544, position 945 to 1744, position 18545 to 19344, position 5745 to 6544, position 11345 to 12144, position 32945 to 33744, position 8945 to 9744, position 3345 to 4144, position 26545 to 27344, position 28145 to 28944, position 25745 to 26544, position 6545 to 7344, position 15345 to 16144, position 4145 to 4944, position 8145 to 8944, position 31345 to 32144, position 2545 to 3344, position 7345 to 8144, position 12145 to 12944, position 20945 to 21744, position 13745 to 14544, position 4945 to 5744, or position 23345 to 24144.

4. Oligonucleotide according to any one of claims 1 to 3, wherein the oligonucleotide comprises, SEQ ID NO.100, SEQ ID NO.42, SEQ ID NO.78, SEQ ID NO.31, SEQ ID NO.97, SEQ ID NO.22, SEQ ID NO.24, SEQ ID NO.28, SEQ ID NO.16, SEQ ID NO.61, SEQ ID NO.99, SEQ ID NO.29, SEQ ID NO.45, SEQ ID NO.15, SEQ ID NO.30, SEQ ID NO.83, SEQ ID NO.41, SEQ ID NO.5, SEQ ID NO.85, SEQ ID NO.74, SEQ ID NO.26, SEQ ID NO.62, SEQ ID NO.87, SEQ ID NO.39, SEQ ID NO.4, SEQ ID NO.60, SEQ ID NO.48, SEQ ID NO.52, SEQ ID NO.63, SEQ ID NO.104, SEQ ID NO.67, SEQ ID NO.92, SEQ ID NO.14, SEQ ID NO.103, SEQ ID NO.58, SEQ ID NO.20, SEQ ID NO.70, SEQ ID NO.8, SEQ ID NO.10, SEQ ID NO.47, SEQ ID NO.88, SEQ ID NO.59, SEQ ID NO.33, SEQ ID NO.75, SEQ ID NO.34, SEQ ID NO.9, SEQ ID NO.56, SEQ ID NO.96, SEQ ID NO.53, SEQ ID NO.18, SEQ ID NO.32, SEQ ID NO.44, SEQ ID NO.84, SEQ ID NO.13, SEQ ID NO.69, SEQ ID NO.90, SEQ ID NO.57, SEQ ID NO.89, SEQ ID NO.82, SEQ ID NO.102, SEQ ID NO.3, SEQ ID NO.12, SEQ ID NO.17, SEQ ID NO.55, SEQ ID NO.25, SEQ ID NO.23, SEQ ID NO.21, SEQ ID NO.93, SEQ ID NO.66, SEQ ID NO.91, SEQ ID NO.51, SEQ ID NO.19, SEQ ID NO.71, SEQ ID NO.80, SEQ ID NO.54, SEQ ID NO.73, SEQ ID NO.76, SEQ ID NO.27, SEQ ID NO.101, SEQ ID NO.6, SEQ ID NO.38, SEQ ID NO.50, SEQ ID NO.35, SEQ ID NO.43, SEQ ID NO.79, SEQ ID NO.72, SEQ ID NO.94, SEQ ID NO.68, SEQ ID NO.7, SEQ ID NO.98, SEQ ID NO.64, SEQ ID NO.77, SEQ ID NO.95, SEQ ID NO.49, SEQ ID NO.65, SEQ ID NO.11, SEQ ID NO.37, SEQ ID NO.40, SEQ ID NO.46, SEQ ID NO.36, SEQ ID NO.81 or a combination thereof.

5. Oligonucleotide according to any one of claims 1 to 4, wherein the oligonucleotide is selected from the group consisting of
+G*+A*+A*T*T*G*T*G*T*T*C*A*A*C*+C*+G*+T (A43004H, SEQ ID NO.100),
+A*+G*+G*A*T*T*C*G*A*G*T*A*C*+A*+G*+T (A43045H, SEQ ID NO.42),
+C*+T*+C*T*T*G*T*T*C*T*G*G*T*T*+C*+C*+G (A43081Hi, SEQ ID NO.78),
+G*+T*+T*G*A*T*C*A*A*G*C*A*C*C*+T*+T*+G (A43034H, SEQ ID NO.31),
+C*+C*+T*A*T*C*T*C*C*G*G*C*T*A*+C*+T*+T (A43100Hi, SEQ ID NO.97),
+T*+C*+A*+A*A*T*T*A*G*C*C*A*C*T*C*+G*+C (A43025H, SEQ ID NO.22),
+G*+T*+T*G*T*C*A*T*T*C*A*G*A*C*+G*+G*+A (A43027H, SEQ ID NO.24),
+C*+C*+T*T*T*G*C*T*A*A*T*A*T*C*+G*+A*+T (A43031H, SEQ ID NO.28),
+A*+A*+T*C*G*T*G*A*G*T*G*C*T*T*+G*+T*+T (A43017H, SEQ ID NO.16),
+C*+G*+A*T*C*C*A*A*G*C*G*T*A*+T*+T*+T (A43064Hi, SEQ ID NO.61),
+T*+T*+C*G*T*G*T*A*C*T*T*A*A*C*+T*+T*+G (A43102Hi, SEQ ID NO.99),
+T*+C*+G*A*T*T*C*C*A*A*A*C*A*T*+C*+A*+C (A43032H, SEQ ID NO.29),
+C*+T*+A*A*C*T*G*T*A*C*C*G*C*+T*+T*+C (A43048Hi, SEQ ID NO.45),
+G*+G*+T*T*G*T*G*C*A*G*C*A*T*A*+T*+G*+C (A43016H, SEQ ID NO.15),
+T*+A*+G*A*C*C*T*G*T*C*A*C*C*T*+T*+G*+A (A43033H, SEQ ID NO.30),
+A*+C*+C*A*T*G*T*C*T*A*A*T*A*C*+T*+A*+T (A43086Hi, SEQ ID NO.83),
+G*+G*+T*C*C*T*A*T*C*A*A*C*C*A*+G*+A*+T (A43044Hi, SEQ ID NO.41),
+A*+C*+A*G*G*T*C*T*T*C*G*G*C*T*+T*+C*+G (A43003H, SEQ ID NO.5),
+T*+G*+T*C*T*G*C*T*A*C*G*T*G*A*+T*+G*+C (A43088Hi, SEQ ID NO.85),
+T*+T*+G*A*C*G*T*T*C*T*A*G*T*G*+C*+T*+C (A43077Hi, SEQ ID NO.74),
+T*+C*+G*A*T*G*G*A*G*G*C*A*T*A*+A*+G*+G (A43029H, SEQ ID NO.26),
+C*+C*+G*A*C*T*C*T*G*T*G*T*A*T*+C*+T*+G (A43065Hi, SEQ ID NO.62),
+G*+A*+A*C*G*T*G*C*C*T*A*A*C*+C*+A*+T (A43090Hi, SEQ ID NO.87),
+C*+G*+A*C*T*T*G*T*A*C*C*T*G*T*+G*+T*+G (A43042Hi, SEQ ID NO.39),
+C*+T*+T*C*G*G*C*T*T*C*G*T*G*G*+T*+T*+A (A43002H, SEQ ID NO.4),
+G*+T*+A*A*A*G*C*A*C*G*G*T*C*T*+G*+A*+T (A43063Hi, SEQ ID NO.60),
+T*+G*+A*T*A*A*C*A*C*A*C*A*C*G*+G*+A*+T (A43051Hi, SEQ ID NO.48),
+A*+G*+T*T*G*T*G*T*A*A*G*T*A*T*+C*+A*+G (A43055Hi, SEQ ID NO.52),
+G*+C*+C*T*A*A*C*T*T*G*C*C*G*A*+C*+T*+T (A43066Hi, SEQ ID NO.63),
+G*+T*+C*C*T*T*G*T*G*T*A*A*T*A*+T*+C*+G (A43019H, SEQ ID NO.104),
+T*+T*+T*A*G*T*C*C*T*C*T*A*C*T*+C*+C*+G (A43070Hi, SEQ ID NO.67),
+G*+T*+T*T*G*A*A*G*G*T*T*C*A*C*+G*+T*+A (A43095Hi, SEQ ID NO.92),
+A*+G*+G*A*A*G*T*C*G*T*C*C*A*T*+T*+G*+T (A43015H, SEQ ID NO.14),
+A*+C*+T*A*T*C*T*C*T*C*G*C*T*T*+C*+A*+T (A43018H, SEQ ID NO.103),
+C*+T*+T*A*C*G*A*C*A*T*G*T*A*C*+C*+A*+C (A43061Hi, SEQ ID NO.58),
+T*+C*+G*G*A*A*C*A*G*G*T*A*C*A*+T*+T*+T (A43023H, SEQ ID NO.20),
+C*+A*+C*C*T*T*A*C*C*T*A*G*G*C*+A*+T*+A (A43073Hi, SEQ ID NO.70),
+T*+G*+C*C*G*A*C*C*T*C*A*G*A*T*+C*+T*+C (A43007H, SEQ ID NO.8),
+T*+C*+A*A*T*C*A*A*C*T*G*G*C*C*+G*+C*+G (A43009H, SEQ ID NO.10),
+T*+T*+G*A*C*T*A*C*G*C*A*T*G*T*+G*+A*+C (A43050Hi, SEQ ID NO.47),
+C*+G*+T*A*G*T*G*C*T*G*C*A*A*C*+C*+A*+T (A43091Hi, SEQ ID NO.88),
+G*+T*+C*T*C*T*C*T*T*A*C*G*A*C*+A*+T*+G (A43062Hi, SEQ ID NO.59),
+G*+A*+G*T*G*T*G*T*A*A*A*T*C*T*+A*+G*+C (A43036H, SEQ ID NO.33),
+G*+A*+T*T*G*T*C*T*A*T*A*T*G*C*+G*+A*+A (A43078Hi, SEQ ID NO.75),
+T*+C*+T*T*A*C*C*G*C*T*T*A*A*T*+G*+G*+A (A43037Hi, SEQ ID NO.34),
+C*+A*+A*C*T*G*G*C*C*G*C*G*G*C*T*G*+T (A43008H, SEQ ID NO.9),
+A*+A*+G*T*C*G*T*T*G*T*C*C*T*T*+A*+G*+A (A43059Hi, SEQ ID NO.56),
+G*+A*+G*C*T*T*A*G*C*C*A*A*T*C*+A*+A*+C (A43099Hi, SEQ ID NO.96),
+T*+A*+G*G*A*G*T*C*A*G*A*T*G*A*+G*+T*+A (A43056Hi, SEQ ID NO.53),
+G*+A*+G*C*A*G*T*G*G*C*C*T*T*A*+C*+A*+T (A43021H, SEQ ID NO.18),
+A*+T*+G*+A*G*T*T*T*C*T*A*T*C*+A*+G*+G*+C (A43035H, SEQ ID NO.32),
+G*+T*+T*T*A*C*T*T*C*C*G*A*A*G*+C*+T*+A (A43047Hi, SEQ ID NO.44),
+T*+A*+G*G*C*C*T*T*G*T*A*G*T*T*+G*+A*+G (A43087Hi, SEQ ID NO.84),
+T*+C*+G*T*C*C*A*T*T*G*T*C*A*C*+C*+T*+T (A43014H, SEQ ID NO.13),
+T*+T*+A*C*C*G*C*C*T*A*C*T*G*T*+A*+T*+G (A43072Hi, SEQ ID NO.69),
+G*+A*+G*A*C*T*T*G*A*G*C*T*C*C*+T*+A*+G (A43093Hi, SEQ ID NO.90),
+T*+C*+G*A*T*C*C*A*A*G*C*G*T*A*+T*+T*+T (A43060Hi, SEQ ID NO.57),
+G*+A*+T*C*T*G*A*T*T*C*C*A*T*G*+T*+G*+C (A43092Hi, SEQ ID NO.89),
+A*+C*+T*G*A*T*C*T*A*A*T*A*T*C*+A*+T*+C (A43085Hi, SEQ ID NO.82),
+T*+A*+A*G*G*A*T*C*C*T*G*A*A*+G*+T*+C*+G (A43013H, SEQ ID NO.102),
+G*+A*+A*G*A*G*C*T*T*G*A*C*A*T*+C*+G*+T (A43001H, SEQ ID NO.3),
+A*+T*+A*+C*A*A*A*G*A*A*C*T*T*C*+T*+C*+G (A43012H, SEQ ID NO.12),
+C*+C*+T*T*C*A*T*G*T*T*T*A*G*C*+T*+G*+C (A43020H, SEQ ID NO.17),
+C*+A*+T*T*A*C*C*A*T*A*C*A*A*C*+G*+C*+C (A43058Hi, SEQ ID NO.55),
+T*+A*+G*G*A*G*G*T*C*C*A*A*G*T*+G*+T*+T (A43028H, SEQ ID NO.25),
+C*+A*+T*C*A*T*T*G*A*T*A*C*G*G*+C*+T*+C (A43026H, SEQ ID NO.23),
+G*+A*+T*C*G*G*A*A*C*A*G*G*T*A*+C*+A*+T (A43024H, SEQ ID NO.21),
+T*+G*+T*G*A*C*A*A*G*G*T*A*A*C*+T*+C*+A (A43096Hi, SEQ ID NO.93),
+C*+T*+A*C*T*C*C*G*C*T*G*A*A*G*+G*+T*+C (A43069Hi, SEQ ID NO.66),
+G*+C*+A*G*C*G*C*A*A*T*T*C*T*G*+A*+T*+C (A43094Hi, SEQ ID NO.91),
+T*+A*+C*A*T*A*C*A*G*A*A*A*G*C*+G*+G*+C (A43054Hi, SEQ ID NO.51),
+C*+T*+C*C*A*G*T*C*G*G*T*A*C*T*C*+C*+A (A43022H, SEQ ID NO.19),
+A*+T*+G*G*A*C*A*C*C*T*T*A*C*C*+T*+A*+G (A43074Hi, SEQ ID NO.71),
+A*+A*+G*T*G*A*T*G*C*G*G*T*A*G*+T*+A*+T (A43083Hi, SEQ ID NO.80),
+A*+A*+C*G*C*C*A*A*T*G*G*A*T*G*+C*+A*+T (A43057Hi, SEQ ID NO.54),
+G*+C*+A*C*G*C*T*G*T*T*T*G*G*T*+A*+T*+T (A43076Hi, SEQ ID NO.73),
+C*+T*+A*C*A*T*C*T*G*G*C*G*G*+A*+A*+C (A43079Hi, SEQ ID NO.76),
+A*+T*+A*T*C*G*A*T*G*G*A*G*G*C*+A*+T*+A (A43030H, SEQ ID NO.27),
+G*+G*C*C*T*G*G*T*C*C*A*C*C*A*T*T*+G (A43011H, SEQ ID NO.101),
+G*+C*+A*+T*+T*C*T*T*G*T*G*G*A*T*+T*+A*+T (A43005H, SEQ ID NO.6),
+A*+A*+G*C*T*C*T*A*G*A*G*A*C*C*+A*+C*+G (A43041Hi, SEQ ID NO.38),
+G*+A*+T*C*T*G*C*C*G*A*G*A*C*+T*+A*+A (A43053Hi, SEQ ID NO.50),
+T*+T*+A*G*G*T*G*A*A*G*C*G*T*T*+C*+C*+T (A43038Hi, SEQ ID NO.35),
+C*+A*+C*G*T*T*G*T*T*T*G*A*G*A*+T*+A*+A (A43046Hi, SEQ ID NO.43),
+G*+G*+A*C*C*G*T*C*T*A*T*G*C*C*+A*+T*+A (A43082Hi, SEQ ID NO.79),
+G*+A*+C*A*A*T*G*T*G*G*A*A*G*T*+C*+G*+G (A43075Hi, SEQ ID NO.72),
+C*+C*+T*A*T*C*T*C*C*G*G*C*T*A*+C*+T*+T (A43097Hi, SEQ ID NO.94),
+T*+T*+A*A*G*C*C*T*C*A*C*T*C*T*+A*+G*+T (A43071Hi, SEQ ID NO.68),
+G*+C*+C*T*C*T*C*A*T*T*G*T*A*G*+T*+C*+T (A43006H, SEQ ID NO.7),
+C*+G*+A*G*G*T*A*T*G*G*C*T*G*T*+G*+G*+A (A43101Hi, SEQ ID NO.98),
+T*+A*+G*C*T*G*C*C*G*T*G*A*C*T*+T*+A*+G (A43067Hi, SEQ ID NO.64),
+T*+C*+T*C*C*G*C*T*A*C*A*A*G*A*+A*+C*+T (A43080Hi, SEQ ID NO.77),
+A*+A*+G*A*G*C*C*A*A*G*T*A*C*A*+C*+G*+A (A43098Hi, SEQ ID NO.95),
+A*+T*+T*A*A*A*G*A*C*G*G*C*C*T*+C*+T*+G (A43052Hi, SEQ ID NO.49),
+A*+C*+T*C*G*T*A*A*G*A*C*A*C*+A*+T*+T (A43068Hi, SEQ ID NO.65),
+G*+A*+G*G*C*A*T*C*C*A*A*T*T*G*G*A*+C (A43010H, SEQ ID NO.11),
+T*+A*+A*G*T*G*T*G*G*T*A*G*G*C*+T*+A*+G (A43040Hi, SEQ ID NO.37),
+A*+G*+C*T*T*T*G*G*A*A*C*T*A*G*+T*+T*+T (A43043Hi, SEQ ID NO.40),
+G*+T*+A*A*T*G*A*G*C*C*A*C*A*C*+A*+A*+G (A43049Hi, SEQ ID NO.46),
+A*+A*+T*G*A*T*G*C*G*A*G*A*G*G*+A*+T*+C (A43039Hi, SEQ ID NO.36),
+T*+C*+A*T*C*G*A*C*C*A*A*A*T*G*+C*+T*+C (A43084Hi, SEQ ID NO.81) or a combination thereof, wherein + indicates an LNA nucleotide and * indicates a phosphorothioate (PTO) linkage between the nucleotides.

6. Oligonucleotide according to any one of claims 1 to 5, wherein the oligonucleotide inhibits the expression of ACE2, ACE2 mRNA, ACE2 pre-mRNA or a combination thereof at a nanomolar or micromolar concentration.

7. Pharmaceutical composition comprising an oligonucleotide according to any one of claims 1 to 6 and a pharmaceutically acceptable carrier, excipient, dilutant, stimulant such as an adjuvant, or a combination thereof.

8. Pharmaceutical composition of claim 7, further comprising an active agent such as an antiviral active agent, an immune stimulating agent, disease specific agent or an agent that reverses infection-mediated immunosuppression or a combination thereof.

9. Pharmaceutical composition of claim 8, wherein the antiviral active agent, the immune stimulating agent, disease specific agent or the agent that reverses infection-mediated immunosuppression is selected from the group consisting of another oligonucleotide, an antibody, a small molecule, a lipid and/or a therapeutic such as a nucleoside analogue, a nucleotide analogue, a protease inhibitor, an ACE2 blocking peptide, an ACE2 fusion protein, a recombinant ACE2 such as Remdesivir, Umifenovir, Favipiravir, Chloroquine, Hydroxychloroquine, Dexamethasone, Lopinavir, Ritonavir, Darunavir, APN01, Favilavir, Molnupiravir, SNG001, Tocilizumab, Anakinra or a combination thereof.

10. Oligonucleotide according to any one of claims 1 to 6 or pharmaceutical composition according to any one of claims 7 to 9 for use in a method of preventing and/or treating a viral disease.

11. Oligonucleotide according to any one of claims 1 to 6 or pharmaceutical composition according to any one of claims 7 to 9 for use in combination with vaccination to prevent a viral disease.

12. Oligonucleotide or pharmaceutical composition for use according to claim 10 or 11, wherein the viral disease is caused by a corona virus such as the severe acute respiratory syndrome coronavirus (SARS-CoV), the severe acute respiratory syndrome coronavirus 2 (SARS-CoV-2) or human corona virus NL63 (HCoV-NL63).

13. Oligonucleotide or pharmaceutical composition for use according to any one of claims 10 to 12, wherein the viral disease is Coronavirus Disease 2019 (COVID-19), Severe Acute Respiratory Syndrome (SARS) or Middle East Respiratory Syndrome (MERS).

14. Oligonucleotide or pharmaceutical composition for use according to any one of claims 10 to 13, wherein the oligonucleotide and/or the pharmaceutical composition is administered locally or systemically.

15. Kit comprising an oligonucleotide according to any one of claims 1 to 6 or pharmaceutical composition according to any one of claims 7 to 9 and optionally technical instructions providing information on administration and/or dosage of the oligonucleotide or pharmaceutical composition.
